(19) United States
(12) Patent Application Publication
(10) Pub. No.: US 2021/0162028 A1
(43) Pub. Date: Jun. 3, 2021

(54) Methods for Inducing Intermittent Fasting and Modulating Autophagy

(71) Applicants: The Hong Kong Polytechnic University, Hong Kong (CN); The Chinese University of Hong Kong, Hong Kong (CN)

(72) Inventors: Yun Chung LEUNG, Hong Kong (CN); Alisa Sau-wun SHUM, Hong Kong (CN); Man Yuen LEE, Hong Kong (CN)

(21) Appl. No.: 17/247,153

(22) Filed: Dec. 2, 2020

Related U.S. Application Data
(60) Provisional application No. 62/942,354, filed on Dec. 2, 2019.

(51) Int. Cl. A61K 38/50; A61K 38/51; A61K 31/353; A61K 31/436

(52) U.S. Cl. CPC A61K 38/50; A61K 38/51; A61K 45/06; A61K 31/436; A61K 31/353

(57) ABSTRACT

The present disclosure provides methods for inducing intermittent fasting and modulating autophagy in cells or organs in a subject via periodic administration of arginine-depleting agents. Induction of intermittent fasting and modulation of autophagy are useful in preventing and/or treating diseases, including those associated with deficits in autophagy, promoting the clearance of intracellular pathogens and protein aggregates, and promoting regeneration and longevity. The methods can be used alone or in combination with other agents to enhance intermittent fasting and autophagy activity to potentiate the health benefit(s).

### CROSS-REFERENCE TO RELATED APPLICATIONS

This application claims priority from U.S. Provisional Patent Application No. 62/942,354, filed on Dec. 2, 2019, which is incorporated herein by reference in its entirety.

### TECHNICAL FIELD

The present disclosure relates to methods for inducing intermittent fasting and modulating autophagy in cells or organs in a subject via periodic administration of arginine-depleting agents.

### BACKGROUND

Intermittent fasting (intermittent energy restriction) has been shown to bring many health benefits. It can help prevent and treat a large variety of diseases. For example, intermittent fasting protects against diabetes, cancers, heart disease, and neurodegeneration. It can also help reduce obesity, hypertension, asthma, and rheumatoid arthritis. It can promote multi-system regeneration, enhances cognitive performance and healthspan [Brandhorst et al. Cell Metab. 2015; 22(1):86 -99] and delay aging.

Intermittent fasting is an umbrella term for various feeding patterns that cycle between voluntary fasting (or reduced calories intake) and non-fasting over a given period. There are different methods of intermittent fasting, which are mainly achieved via food deprivation and/or consumption of a calorie restricted diet. For example, one method is alternate-day fasting, which involves alternating between a 24-hour fast day when the subject eats less than 25% of usual energy needs, followed by a 24-hour non-fasting feast day period. Another method is periodic fasting, which involves any period of consecutive fasting of more than 24 hours, such as 5:2 diet, where there are 2 fast days per week. During the fasting days, the subject has very low or about 25% of regular daily caloric intake. Another method is time-restricted fasting, which involves eating only during a certain number of hours each day, such as 16:8 diet (16 fasting hours cycled by 8 non-fasting hours). Some studies achieved intermittent fasting via continuous feeding on a diet that mimics fasting (fasting mimicking diet “FMD”) for several days, and the FMD cycle is repeated at regular intervals [Brandhorst et al. Cell Metab. 2015; 22(1):86-99]. While these feeding patterns and/or diets can bring health benefits, long term compliance may be difficult to achieve. A number of agents can also exert health benefits via reduction of food intake. For example, celastrol-induced weight loss is driven by hypophagia [Pfuhlmann et al. Diabetes. 2018; 67(10:2456-2465]. However, many of these agents have short half-life and are required to be administered daily to achieve therapeutic effects.

Cells can undergo macro-autophagy (referred to as autophagy). During autophagy, the cell consumes parts of itself in a regulated manner, which involves delivery of cellular components to the lysosome for degradation via a double membrane-bound structure. Autophagy occurs constitutively at low levels to balance the constant synthesis of biomolecules. It maintains cellular integrity by degrading long-lived intracellular proteins and damaged organelles, and recycling their components into metabolic precursors. Autophagy is recognized as a critical process for maintaining cellular homeostasis as well as for responding to stress. When a cell is exposed to stress, such as nutrient deficiency or fasting, autophagy is strongly upregulated. This upregulation increases sequestration and degradation of a large number and variety of substrates, including the degradation of entire organelles, releasing macromolecules back into the cytosol to supply essential metabolic reactions and generate energy. Recent studies have shown that autophagy also clears a wide range of intracellular pathogens. Impaired autophagy is related to many types of diseases, including cancer, infectious, neurodegenerative, inflammatory, age-associated, and metabolic diseases. With new findings, knowledge and information on the relationship among intermittent fasting, autophagy and diseases, it is possible to design effective therapeutic methods and strategies. Agents that can modulate autophagy are potential therapeutics for treatment of many diseases [Levine et al., J Clin Invest. 2015; 125(1):14-24].

Autophagy is a tightly regulated catabolic process in which damaged proteins and organelles are delivered to the lysosome and degraded to release free amino acids into the cytoplasm. Autophagy is specifically activated in response to amino acid starvation via the mammalian target of rapamycin (mTOR) complex 1 (mTORC1), which is the central metabolic sensor of the cell. mTORC1 is the key hub coordinating the availability of amino acids and autophagy [Carroll et al., Amino Acids. 2015; 47(10):2065-2088]. It inhibits autophagy induction when materials are abundant. When cells are starved of these nutrients, mTORC1 is inactivated, promoting an increase in autophagy.

Obesity-induced diabetes is characterized by hyperglycemia, insulin resistance, and progressive beta cell failure. In islets of mice with obesity-induced diabetes, Liu et al. [Autophagy. 2017; 13(11):1952-1968] observed increased beta cell death and impaired autophagic flux. They found that intermittent fasting stimulates autophagic flux to ameliorate obesity-induced diabetes. They showed that despite continued high-fat intake, intermittent fasting restores autophagic flux in islets and improves glucose tolerance by enhancing glucose-stimulated insulin secretion, beta cell survival, and nuclear expression of NEUROG3, a marker of pancreatic regeneration. They found that intermittent fasting does not rescue beta-cell death or induce NEUROG3 expression in obese mice with lysosomal dysfunction secondary to deficiency of the lysosomal membrane protein, LAMP2 or haplo-insufficiency of BECN1/Beclin 1, a protein critical for autophagosome formation. Thus, intermittent fasting can preserve organelle quality via the autophagy-lysosome pathway to enhance beta cell survival and it can stimulate markers of regeneration in obesity-induced diabetes.

There is thus a need to develop new methods for inducing an intermittent fasting state and/or modulating autophagy subject that overcomes at least some of the disadvantages presented above.

### SUMMARY

Disclosed herein is the use of periodic administration of long-acting arginine-depleting agents to induce cyclic occurrences of intermittent fasting and autophagy to bring health benefits to a subject. The arginine-depleting agent can be arginase, arginine deiminase or arginine decarboxylase. The circulating half-life of these enzymes can be extended by using any conventional method known in the art, such as by PEGylation, fusion with albumin binding domain or human serum albumin, or a human IgG Fc domain. The arginine-depleting agent can be administered alone, or in combination with other methods or agents to enhance intermittent fasting and autophagy, e.g. metformin and its analogue, retinoid and its derivatives, green tea polyphenol (−)-epigallocatechin-3-gallate (EGCG) and its derivatives, and rapamycin and its analogue.

In a first aspect provided herein is a method of inducing intermittent fasting, modulating autophagy, or inducing intermittent fasting and modulating autophagy in a subject in need thereof comprising the step of administering a therapeutically effective amount of an arginine depleting agent to the subject.

In certain embodiments, inducing intermittent fasting, modulating autophagy, or inducing intermittent fasting and modulating autophagy in the subject results in treatment of at least one autophagy related or intermittent fasting related disease or health condition selected from the group consisting of increasing the longevity of the subject, a symptom of aging or preventing an age related disease, and promoting cellular regeneration.

In certain embodiments, the arginine concentration in the subject's serum is maintained below 50 μM, below 25 μM, below 20 μM, below 10 μM, or below 5 μM.

In certain embodiments, the arginine depleting agent is an arginase protein, an arginine deiminase protein, or an arginine decarboxylase protein.

In certain embodiments, the arginase protein, arginine deiminase protein, or arginine decarboxylase protein further comprises one or more polyethylene glycol (PEG) groups.

In certain embodiments, arginase protein comprises a polypeptide having SEQ ID NO: 101, SEQ ID NO: 102, SEQ ID NO: 103, or SEQ ID NO: 104.

In certain embodiments, the arginase protein, arginine deiminase protein, or arginine decarboxylase protein further comprises an albumin binding domain or human serum albumin, or a human IgG Fc domain.

In certain embodiments, the arginine depleting agent is a fusion protein comprising an ABD polypeptide and an arginase polypeptide; an ABD polypeptide and an arginine deiminase polypeptide; or an ABD polypeptide and an arginine decarboxylase polypeptide.

In certain embodiments, the arginine depleting agent comprises a polypeptide having at least 98% sequence homology with SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 75, SEQ ID NO: 107, or SEQ ID NO: 76.

In certain embodiments, the arginine depleting agent is co-administered with a therapeutically effective amount of an autophagy inducing agent.

In certain embodiments, the autophagy inducing agent is selected from the group consisting of a retinoid derivative, an (−)-epigallocatechin-3-gallate (EGCG) derivative, a green tea catechin, and a rapamycin derivative.

In certain embodiments, the autophagy inducing agent is selected from the group consisting of carbamazepine, clonidin, lithium, metformin, rapamycin (and rapalogs), rilmenidine, sodium valproate, verapamil, trifluoperazine, statins, tyrosine kinase inhibitors, BH3 mimetics, caffeine, omega-3 polyunsaturated fatty acids, resveratrol, spermidine, vitamin D, trehalose, polyphenol(−)-epigallocatechin-3-gallate and combinations thereof.

In certain embodiments, the arginine depleting agent is co-administered with a therapeutically effective amount of a glucose lowering agent.

In certain embodiments, the glucose lowering agent is an alpha-glucosidase inhibitor, a biguanide, bile acid sequestrant, a dopamine-2 agonist, a dipeptidyl peptidase 4 (DPP-4) inhibitor, a meglitinide, a sodium-glucose transport protein 2 (SGLT2) inhibitor, a sulfonylurea, a thiazolidinedione, or a combination thereof.

In certain embodiments, the biguanide is metformin; the alpha-glucosidase inhibitor is acarbose or miglitol; the bile acid sequestrant is colesevelam; the dopamine-2 agonist is bromocriptine; the DPP-4 inhibitor is alogliptin, linagliptin, saxagliptin, or sitagliptin; the meglitinide is nateglinide or repaglinide; the SGLT2 inhibitor is canagliflozin, dapagliflozin, or empagliflozin; the sulfonylureas ischlorpropamide, glimepiride, glipizide, or glyburide; and the thiazolidinedione is rosiglitazone or pioglitazone.

In certain embodiments, the arginine depleting agent is co-administered with a therapeutically effective amount of a retinoid derivative.

In certain embodiments, the retinoid derivative is acitretin, alitretinoin bexarotene, isotretinoin, retinol, retinoic acid, or a pharmaceutically acceptable salt thereof.

In certain embodiments, the retinoid derivative is retinoic acid.

In certain embodiments, the arginine depleting agent is co-administered with a therapeutically effective amount of an (−)-epigallocatechin-3-gallate (EGCG) derivative, a green tea catechin or a pharmaceutically acceptable salt or product thereof.

In certain embodiments, the EGCG derivative is EGCG or pharmaceutically acceptable salt thereof or EGCG peracetate.

In certain embodiments, the arginine depleting agent is co-administered with a therapeutically effective amount of a rapamycin derivative or pharmaceutically acceptable salt thereof.

### BRIEF DESCRIPTION OF THE DRAWINGS

The above and other objects and features of the present disclosure will become apparent from the following description of the invention, when taken in conjunction with the accompanying drawings, in which:

FIG. 1 illustrates that C57BL/6J male mice with pre-existing obesity, induced by feeding a high-fat diet (HFD) from 5-week old for 12 weeks, referred as diet-induced obese (DIO) mice, exhibited repetitive 7-day intermittent fasting cycles consisted of periods of fasting and refeeding when administered with about 600 U N-ABD094-rhArg (SEQ ID NO: 50) (rhArg) once a week for 34 weeks. (A) Patterns of food intake of 3 groups of mice: DIO mice fed with HFD and injected with rhArg [HFD (rhArg) group]; DIO mice fed with HFD and injected with saline (vehicle) [HFD (vehicle) group]; mice fed with an ordinary chow diet (CD) and injected with vehicle [CD (vehicle) group] served as the lean control. (B) Average food intake on each day of a 7-day intermittent fasting cycle. Day 0 represented the day of rhArg injection, which was Day 7 of the previous cycle. Day 7 represented the 7^{th} day after rhArg injection on Day 0 and was Day 0 of the next cycle. (C) Total food intake per week of HFD (rhArg) group was about 30% less than that of HFD (vehicle) group. *P<0.05, Mann-Whitney U test. Data are expressed as mean±SEM, n=8-12 for each group.

FIG. 2 illustrates that administration of N-ABD094-rhArg (SEQ ID NO: 50) to DIO male mice fed a HFD [HFD (rhArg) group] in FIG. 1 could induce substantial weight loss within 6-7 weeks of treatment, and the bodyweight was maintained relatively constant at around 30 g for the rest of the treatment period. (A) Change in bodyweight over the 34-week of treatment period. (B) Representative images of the 3 groups of mice at the end of the treatment period.

FIG. 3 illustrates that C57BL/6J male mice with pre-existing HFD-induced obesity exhibited repetitive 7-day intermittent fasting cycles when administered with about 600 U N-ABD094-rhArg (SEQ ID NO: 50) (rhArg) once a week for 49 weeks. (A) Patterns of food intake of 3 groups of mice: DIO mice fed with HFD and injected with rhArg [HFD (rhArg) group]; DIO mice fed with HFD and injected with saline (vehicle) [HFD (vehicle) group]; mice fed with an ordinary chow diet (CD) and injected with vehicle [CD (vehicle) group] served as the lean control. (B) Average food intake on each day of a 7-day intermittent fasting cycle. (C) Total food intake per week of HFD (rhArg) group was about 29% less than that of HFD (vehicle) group. *P<0.05, Mann-Whitney U test. Data are expressed as mean±SEM, n=5 for CD (vehicle) group; n=9 for HFD (vehicle) and HFD (rhArg) groups.

FIG. 4 illustrates that administration of N-ABD094-rhArg (SEQ ID NO: 50) to DIO male mice fed a HFD [HFD (rhArg) group] in FIG. 3 for 49 weeks could induce substantial weight loss within 6-7 weeks and the bodyweight was maintained relatively constant at around 30 g for the rest of the treatment period.

FIG. 5 illustrates that anti-rhArg antibodies detected in the serum of HFD (rhArg) group of mice in FIG. 1 did not have neutralizing activities. (A, B) Anti-rhArg antibody tiers in the serum taken from mice at 5 week (A) and 23 week (B) after rhArg treatment were similar. (C) The serum taken at 23 week after rhArg treatment was incubated with rhArg and did not show any effects on neutralizing the enzymatic activity of rhArg.

FIG. 6 illustrates that administration of N-ABD094-rhArg (SEQ ID NO: 50) to DIO male mice fed a HFD [HFD (rhArg) group] in FIG. 1 for 34 weeks could effectively reduce fat mass. (A) Representative images of freshly dissected fat pad at main visceral (perirenal) and subcutaneous (inguinal) white adipose tissue (WAT) depots, and main (interscapular) brown adipose tissue (BAT) depot. (B) The mass of perirenal and inguinal WAT, and interscapular BAT of DIO mice treated with rhArg [HFD (rhArg) group] was markedly reduced in comparison to DIO mice treated with vehicle [HFD (vehicle) group]. *P<0.05, one-way ANOVA followed by Bonferroni test. Data are expressed as mean±SEM.

FIG. 7 illustrates that administration of N-ABD094-rhArg (SEQ ID NO: 50) to DIO male mice fed a HFD [HFD (rhArg) group] in FIG. 1 for 34 weeks could effectively reduce liver mass, and lower serum concentrations of some commonly-used liver damage biomarkers to levels similar to that of the lean control mice [CD (vehicle) group]. (A) Representative images of fresh whole liver of 3 groups of mice. (B) Liver mass. (C) Serum concentrations of alanine transaminase (ALT) and aspartate transaminase (AST). *P<0.05, one-way ANOVA followed by Bonferroni test. Data are expressed as mean±SEM.

FIG. 8 illustrates that administration of N-ABD094-rhArg (SEQ ID NO: 50) to DIO male mice fed a HFD [HFD (rhArg) group] in FIG. 1 for 34 weeks could reduce kidney mass, and lower urine concentrations of a commonly-used kidney damage biomarker to levels similar to that of the lean control mice [CD (vehicle) group]. (A) Kidney mass. (B) Ratio of albumin-to-creatinine in urine. *P<0.05, one-way ANOVA followed by Bonferroni test. Data are expressed as mean±SEM.

FIG. 9 illustrates that administration of N-ABD094-rhArg (SEQ ID NO: 50) to DIO male mice fed a HFD [HFD (rhArg) group] in FIG. 1 for 34 weeks could reduce heart mass, and lower blood pressure and heart rate to levels similar to that of the lean control mice [CD (vehicle) group]. (A) Heart mass. (B-E) Systolic and diastolic blood pressure (B, D) and heart rate (C, E) measured respectively at 12 weeks (B, C) and 27 weeks (D, E) after rhArg treatment by tail-cuff method using Noninvasive Blood Pressure Monitoring System (CODA Scientific). *P<0.05, one-way ANOVA followed by Bonferroni test. Data are expressed as mean±SEM.

FIG. 10 illustrates that administration of N-ABD094-rhArg (SEQ ID NO: 50) to DIO male mice fed a HFD [HFD (rhArg) group] in FIG. 3 could effectively reverse insulin resistance. (A-C) Insulin tolerance test (ITT) was conducted prior to (A) and at 16 weeks (B) and 32 weeks (C) after rhArg treatment. Results of ITT are expressed as area under the curve (AUC). **P<0.05, Mann-Whitney U test; *P<0.05, one-way ANOVA followed by Bonferroni test. Data are expressed as mean±SEM.

FIG. 11 illustrates that administration of N-ABD094-rhArg (SEQ ID NO: 50) to DIO male mice fed a HFD [HFD (rhArg) group] in FIG. 3 could effectively reverse impaired glucose tolerance. (A-C) Glucose tolerance test (GTT) was conducted prior to (A) and at 15 weeks (B) and 31 weeks (C) after rhArg treatment. Results of GTT are expressed as area under the curve (AUC). *P<0.05, one-way ANOVA followed by Bonferroni test. Data are expressed as mean±SEM.

FIG. 12 illustrates that feeding C57BL/6J male mice, with pre-existing HFD-induced obesity, with a predetermined amount of HFD to create an artificial 7-day intermittent fasting cycle [HFD (artificial IF) group], which mimics the pattern of food intake of DIO mice administered once a week with about 600 U N-ABD094-rhArg (SEQ ID NO: 50) [HFD (rhArg) group], for 5 weeks could effectively reduce bodyweight of mice. (A) Food intake pattern over the 5-week period. (B) Total food intake per week of HFD (rhArg) group and HFD (artificial IF) group was 26% and 34% respectively less than that of DIO mice treated with vehicle [HFD (vehicle) group]. (C) Change in bodyweight over the 5-week period. *P<0.05, Mann-Whitney U test. Data are expressed as mean±SEM, n=5 for each group.

FIG. 13 illustrates that DIO male mice subjected to an artificial 7-day intermittent fasting feeding cycle with a HFD [HFD (artificial IF) group] in FIG. 12 for 5 weeks showed marked reduction in fat pad mass of perirenal (visceral) and inguinal (subcutaneous) white adipose tissue (WAT), and interscapular brown adipose tissue (BAT), and also had marked reduction in liver mass in comparison to DIO mice treated with vehicle [HFD (vehicle) group]. The fat pad and liver mass was comparable to that of DIO mice administered with rhArg once a week [HFD (rhArg) group]. *P<0.05, one-way ANOVA followed by Bonferroni test. Data are expressed as mean±SEM.

FIG. 14 illustrates that DIO male mice subjected to an artificial 7-day intermittent fasting feeding cycle with a HFD [HFD (artificial IF) group] in FIG. 12 showed significant improvement in glucose tolerance, but did not show improvement in insulin sensitivity, when compared to DIO mice treated with vehicle [HFD (vehicle) group]. In contrast, DIO mice [HFD (rhArg) group] exhibited significant improvement in insulin sensitivity by 2 weeks after rhArg treatment. (A) Insulin tolerance test (ITT) conducted at 2 weeks and 4 weeks after rhArg treatment. (B) Glucose tolerance test (GTT) conducted at 3 weeks after rhArg treatment. Results of ITT and GTT are expressed as area under the curve (AUC). *P<0.05, one-way ANOVA followed by Bonferroni test. Data are expressed as mean±SEM.

FIG. 15 illustrates that C57BL/6J male mice, with pre-existing HFD-induced obesity, when subjected to reduced daily food intake of a HFD by 30% [HFD (reduced) group] for 5 weeks showed significantly less weight loss in comparison to DIO mice administered once a week with about 600 U N-ABD094-rhArg (SEQ ID NO: 50) [HFD (rhArg) group]. (A) Food intake pattern of vehicle- and rhArg-treated DIO mice fed ad libitum or a fixed amount (2.0 g) of HFD. (B) Total food intake per week of HFD (reduced) group was about 30% less than vehicle-treated DIO mice fed ad libitum with the HFD [HFD (vehicle) group]. (C) Change in bodyweight over the 5-week period. *P<0.05, Mann-Whitney U test. Data are expressed as mean±SEM, n=5 for each group.

FIG. 16 illustrates that DIO male mice subjected to reduced daily food intake of a HFD by 30% [HFD (reduced) group] in FIG. 15 for 5 weeks showed significantly less reduction in fat pad mass of perirenal (visceral) and inguinal (subcutaneous) white adipose tissue (WAT), and interscapular brown adipose tissue (BAT), and the liver mass, in comparison to DIO mice administered once a week with rhArg [HFD (rhArg) group]. *P<0.05, one-way ANOVA followed by Bonferroni test. Data are expressed as mean±SEM, n=5 for each group.

FIG. 17 illustrates that DIO male mice subjected to reduced daily food intake of a HFD by 30% [HFD (reduced) group] showed significant improvement in glucose tolerance similar to DIO mice administered once a week with about 600 U N-ABD094-rhArg (SEQ ID NO: 50) [HFD (rhArg) group]. In contrast, only mice in HFD (rhArg) group, but not HFD (reduced) group showed improvement in insulin sensitivity compared with vehicle-treated DIO mice fed ad libutum [HFD (vehicle) group]. (A) Insulin tolerance test (ITT) conducted after 2 weeks of treatment. (B) Glucose tolerance test (GTT) conducted after 3 weeks of treatment. Results of ITT and GTT are expressed as area under the curve (AUC). *P<0.05, one-way ANOVA followed by Bonferroni test. Data are expressed as mean±SEM, n=5 for each group.

FIG. 18 illustrates that administration of about 250 U PEGylated His-rhArg (SEQ ID NO: 101) once a week for 8 weeks to C57BL/6J male mice with pre-existing HFD-induced obesity [HFD (PEG-rhArg) group] could induce repetitive 7-day intermittent fasting cycles. (A) Patterns of food intake over the 8-week of treatment period. (B) Average food intake on each day of a 7-day intermittent fasting cycle. (C) Total food intake per week of HFD (PEG-rhArg) group was about 28% less than that of vehicle-treated DIO mice [HFD (vehicle) group]. *P<0.05, Mann-Whitney U test. Data are expressed as mean±SEM, n=5 for each group.

FIG. 19 illustrates that administration of PEGylated His-rhArg (SEQ ID NO: 101) to DIO male mice fed a HFD [HFD (PEG-rhArg) group] in FIG. 18 could induce substantial weight loss within 6-7 weeks to a level similar to the vehicle-treated lean control mice fed an ordinary chow diet [CD (vehicle) group]. n=5 for each group.

FIG. 20 illustrates that administration of PEGylated His-rhArg (SEQ ID NO: 101) to DIO male mice fed a HFD [HFD (PEG-rhArg) group] in FIG. 18 could effectively reverse insulin resistance and improve glucose tolerance. (A) Insulin tolerance test (ITT) was conducted at 7 week after rhArg treatment. (B) Glucose tolerance test (GTT) was conducted at 6 week after rhArg treatment. Results of ITT and GTT are expressed as area under the curve (AUC). *P<0.05, one-way ANOVA followed by Bonferroni test. Data are expressed as mean±SEM.

FIG. 21 illustrates that administration of PEGylated His-rhArg (SEQ ID NO: 101) to DIO male mice fed a HFD [HFD (PEG-rhArg) group] in FIG. 18 for 8 weeks could effectively reduce fat mass of perirenal (visceral) and inguinal (subcutaneous) white adipose tissue (WAT), and interscapular brown adipose tissue (BAT), and the liver to a weight comparable with that of the lean control mice [CD (vehicle) group]. The mass of kidney and heart was also significantly reduced in comparison to vehicle-treated DIO mice [HFD (vehicle) group]. *P<0.05, one-way ANOVA followed by Bonferroni test. Data are expressed as mean±SEM.

FIG. 22 illustrates that administration of 50 U N-ABD094-rhArg-Co²⁺ [SEQ ID NO: 50 (cobalt substituted)] once a week for 2 weeks to C57BL/6J male mouse with pre-existing HFD-induced obesity could induce a 7-day intermittent fasting cycle and a concomitant reduction in bodyweight. (A) Pattern of food intake with period of fasting and refeeding in a 7-day intermittent fasting cycle. (B) Change in bodyweight. n=1.

FIG. 23 illustrates that administration of 5 U ADI-ABD (SEQ ID NO: 107) once a week for 2 weeks to C57BL/6J male mouse with pre-existing HFD-induced obesity could induce a 7-day intermittent fasting cycle and a concomitant reduction in bodyweight. (A) Pattern of food intake with period of fasting and refeeding in a 7-day intermittent fasting cycle. (B) Change in bodyweight. n=1.

FIG. 24 illustrates that administration of N-ABD094-rhArg (SEQ ID NO: 50) once a week to DIO male mice fed a HFD [HFD (rhArg) group] in FIG. 3 could significantly reduce the total latency for them to reach the correct exit during the 4 days of training period in the Barnes maze test for spatial learning and memory conducted at 43-44 weeks after rhArg treatment. *P<0.05, one-way ANOVA followed by Bonferroni test. Data are expressed as mean±SEM.

FIG. 25 illustrates that administration of N-ABD094-rhArg (SEQ ID NO: 50) once a week to DIO male mice fed a HFD [HFD (rhArg) group] in FIG. 3 could significantly improve their search mode in the training period on Day 1 in the Barnes maze test for spatial learning and memory conducted at 43-44 weeks after rhArg treatment in comparison to vehicle-treated DIO mice fed a HFD [HFD (vehicle) group] *P<0.05, Jonckheere-Terpstra test.

FIG. 26 illustrates that long-term administration of N-ABD094-rhArg (SEQ ID NO: 50) once a week to DIO male mice fed a HFD [HFD (rhArg) group] in FIG. 3 could improve their short-term memory and long-term memory to levels similar to age-matched control mice fed a chow diet [CD (vehicle) group] in the Barnes maze test conducted at 43-44 weeks after rhArg treatment. (A) Probe trial on Day 5 of Barnes maze test. (A) Probe trial on Day 10 of Barnes maze test.

FIG. 27 illustrates that long-term administration of N-ABD094-rhArg (SEQ ID NO: 50) once a week to DIO male mice fed a HFD [HFD (rhArg) group] in FIG. 3 could significantly improve their neuromuscular strength and coordination to a level comparable to age-matched control mice fed a chow diet [CD (vehicle) group]. (A) Inverted grid hanging test conducted at 42 weeks after treatment. (B) Rotarod test conducted at 30 weeks after treatment. *P<0.05, one-way ANOVA followed by Bonferroni test. Data are expressed as mean±SEM.

FIG. 28 illustrates that administration of N-ABD094-rhArg (SEQ ID NO: 50) once a week to DIO male mice fed a HFD [HFD (rhArg) group] in FIG. 1 for 34 weeks and in FIG. 3 for 49 weeks could effectively prevent the development of liver cancer. (A) Representative images of fresh whole liver collected at 34 weeks after treatment. The size of the liver of HFD (rhArg) group was similar to that of vehicle-treated control mice fed a chow diet [CD (vehicle) group]. The liver of HFD-fed mice treated with vehicle [HFD (vehicle) group] was markedly enlarged with tumor. (B) Frequency of hepatocellular carcinoma in mice at 34 weeks and 49 weeks after treatment. The liver of HFD (rhArg) group was free of tumor.

FIG. 29 illustrates that ICR female mice with pre-existing obesity, induced by feeding a HFD from 5-week old for 12 weeks, exhibited repetitive 7-day intermittent fasting cycles when administered with about 1200 U N-ABD094-rhArg (SEQ ID NO: 50) (rhArg) once a week for 56 weeks. (A) Patterns of food intake of 3 groups of mice: DIO mice fed with HFD and injected with rhArg [HFD (rhArg) group]; DIO mice fed with HFD and injected with saline (vehicle) [HFD (vehicle) group]; mice fed with an ordinary chow diet (CD) and injected with vehicle [CD (vehicle) group] served as the lean control. (B) Average food intake on each day of a 7-day intermittent fasting cycle. (C) Total food intake per week of HFD (rhArg) group was about 14% less than that of HFD (vehicle) group. *P<0.05, Mann-Whitney U test. Data are expressed as mean±SEM, n=5 for Chow group; n=8 for HFD (vehicle) and HFD (rhArg) groups.

FIG. 30 illustrates that administration of N-ABD094-rhArg (SEQ ID NO: 50) to DIO female mice fed a HFD [HFD (rhArg) group] in FIG. 29 for 56 weeks could effectively induce weight loss, with the bodyweight dropped to a level similar to the age-matched control mice fed a chow diet [CD (vehicle) group] within 9-10 weeks, and their bodyweight could be maintained relatively constant at around 35 g for the rest of the treatment period, which was in contrast to vehicle-treated HFD-fed [HFD (vehicle) group] and Chow-fed [CD (vehicle) group] mice that progressively increased in bodyweight over the 56-week of treatment period.

FIG. 31 illustrates that administration of N-ABD094-rhArg (SEQ ID NO: 50) to DIO female mice fed a HFD [HFD (rhArg) group] in FIG. 29 for 56 weeks could markedly reduce the fat mass of the perirenal (visceral) and inguinal (subcutaneous) white adipose tissue (WAT) and interscapular brown adipose tissue (BAT) to a level comparable to age-matched vehicle-treated control mice fed a chow diet [CD (vehicle) group]. The mass of several major organs, including the liver, kidney and heart was also significantly lower than HFD-fed DIO mice treated with vehicle [HFD (vehicle) group]. *P<0.05, one-way ANOVA followed by Bonferroni test. Data are expressed as mean±SEM.

FIG. 32 illustrates that administration of N-ABD094-rhArg (SEQ ID NO: 50) to DIO female mice fed a HFD [HFD (rhArg) group] in FIG. 29 could effectively reverse insulin resistance. (A-C) Insulin tolerance test (ITT) was conducted prior to (A) and at 15 weeks (B) and 31 weeks (C) after rhArg treatment. Results of ITT are expressed as area under the curve (AUC). **P<0.05, Mann-Whitney U test; *P<0.05, one-way ANOVA followed by Bonferroni test. Data are expressed as mean±SEM.

FIG. 33 illustrates that administration of N-ABD094-rhArg (SEQ ID NO: 50) to DIO female mice fed a HFD [HFD (rhArg) group] in FIG. 29 could effectively reverse impaired glucose tolerance. (A-C) Glucose tolerance test (GTT) was conducted prior to (A) and at 16 weeks (B) and 30 weeks (C) after rhArg treatment. Results of GTT are expressed as area under the curve (AUC). **P<0.05, Mann-Whitney U test; *P<0.05, one-way ANOVA followed by Bonferroni test. Data are expressed as mean±SEM.

FIG. 34 illustrates that long-term administration of N-ABD094-rhArg (SEQ ID NO: 50) once a week to DIO female mice fed a HFD [HFD (rhArg) group] in FIG. 29 could significantly improve their neuromuscular strength and coordination to a level comparable to age-matched control mice fed a chow diet [CD (vehicle) group]. (A) Inverted grid hanging test conducted at 54 weeks after treatment. (B) Rotarod test conducted at 55 weeks after treatment. *P<0.05, one-way ANOVA followed by Bonferroni test. Data are expressed as mean±SEM.

FIG. 35 illustrates that long-term administration of N-ABD094-rhArg (SEQ ID NO: 50) once a week to DIO female mice fed a HFD [HFD (rhArg) group] in FIG. 29 for 56 weeks could effectively prevent the development of hepatocellular carcinoma.

FIG. 36 illustrates that C57BL/6J male mice about 16 months of age (equivalent to mid-fifties in humans), with pre-existing obesity induced by feeding a HFD from 5-week old, exhibited repetitive 7-day intermittent fasting cycles when administered with about 600 U N-ABD094-rhArg (SEQ ID NO: 50) (rhArg) once a week for 25 weeks. The response is similar to C57BL/6J DIO male mice in FIG.1, which began treatment with N-ABD094-rhArg (SEQ ID NO: 50) at 4-5 months old (equivalent to mid-twenties in humans). (A) Patterns of food intake of 3 groups of mice: 16-month old DIO mice fed with HFD and injected with rhArg [HFD Old (rhArg) group]; 16-month old DIO mice fed with HFD and injected with saline (vehicle) [HFD Old (vehicle) group]; 5-month old mice fed with an ordinary chow diet (CD) and injected with vehicle [CD Young (vehicle) group] served as the young lean control. (B) Average food intake on each day of a 7-day intermittent fasting cycle. (C) Total food intake per week of HFD Old (rhArg) group was about 31% less than that of HFD Old (vehicle) group. *P<0.05, Mann-Whitney U test. Data are expressed as mean±SEM; n=6 for CD Young (vehicle) and HFD Old (vehicle) group, n=9 for HFD Old (rhArg) group.

FIG. 37 illustrates that administration of N-ABD094-rhArg (SEQ ID NO: 50) to 16-month old DIO male mice fed a HFD [HFD Old (rhArg) group] in FIG. 36 could induce substantial weight loss within 10 weeks of treatment and the bodyweight was maintained relatively constant at around 30 g for the rest of the treatment period.

FIG. 38 illustrates that administration of N-ABD094-rhArg (SEQ ID NO: 50) to 16-month old DIO male mice fed a HFD [HFD (rhArg) group] in FIG. 36 could effectively reverse insulin resistance. (A, B) Insulin tolerance test (ITT) was conducted prior to (A) and at 9 weeks after rhArg treatment (B). Results of ITT are expressed as area under the curve (AUC). **P<0.05, Mann-Whitney U test; *P<0.05, one-way ANOVA followed by Bonferroni test. Data are expressed as mean±SEM.

FIG. 39 illustrates that administration of N-ABD094-rhArg (SEQ ID NO: 50) to 16-month old DIO male mice fed a HFD [HFD (rhArg) group] in FIG. 36 could effectively reverse impaired glucose tolerance. (A, B) Glucose tolerance test (GTT) was conducted prior to (A) and at 12 weeks after rhArg treatment (B). Results of GTT are expressed as area under the curve (AUC). **P<0.05, Mann-Whitney U test; *P<0.05, one-way ANOVA followed by Bonferroni test. Data are expressed as mean±SEM.

FIG. 40 illustrates that C57BL/6J male mice about 17 months of age (equivalent to about mid-fifties in humans) fed an ordinary chow diet exhibited repetitive 7-day intermittent fasting cycles when administered with about 600 U N-ABD094-rhArg (SEQ ID NO: 50) (rhArg) once a week for 21 weeks. (A) Patterns of food intake of 3 groups of mice: 17-month old mice fed a chow diet and injected with rhArg [CD Old (rhArg) group]; 17-month old mice fed a chow diet and injected with saline (vehicle) [CD Old (vehicle) group]; 5-month old male mice (equivalent to about mid-twenties in humans) fed a chow diet and injected with vehicle [CD Young (vehicle) group] served as the young control. (B) Average food intake on each day of a 7-day intermittent fasting cycle. (C) Total food intake per week of CD Old (rhArg) group was about 11% less than that of CD Old (vehicle) group. *P<0.05, Mann-Whitney U test. Data are expressed as mean±SEM; n=7 for CD Young (vehicle) and CD Old (vehicle) group, n=9 for HFD Old (rhArg) group.

FIG. 41 illustrates that administration of N-ABD094-rhArg (SEQ ID NO: 50) to 17-month old male mice fed a chow diet [CD Old (rhArg) group] in FIG. 40 could induce weight loss from 40 g to 30 g within 8 weeks of treatment and the bodyweight was maintained relatively constant at this level for the rest of the treatment period.

FIG. 42 illustrates that administration of N-ABD094-rhArg (SEQ ID NO: 50) to 17-month old male mice fed a chow diet [CD Old (rhArg) group] in FIG. 40 could effectively improve insulin sensitivity. (A, B) Insulin tolerance test (ITT) was conducted prior to (A) and at 13 weeks after rhArg treatment (B). Results of ITT are expressed as area under the curve (AUC). **P<0.05, Mann-Whitney U test; *P<0.05, one-way ANOVA followed by Bonferroni test. Data are expressed as mean±SEM.

FIG. 43 illustrates that administration of N-ABD094-rhArg (SEQ ID NO: 50) to 17-month old male mice fed a chow diet [CD Old (rhArg) group] in FIG. 40 could effectively improve glucose tolerance. (A, B) Glucose tolerance test (GTT) was conducted prior to (A) and at 15 weeks after rhArg treatment (B). Results of GTT are expressed as area under the curve (AUC). **P<0.05, Mann-Whitney U test; *P<0.05, one-way ANOVA followed by Bonferroni test. Data are expressed as mean±SEM.

FIG. 44 illustrates that at the end of 21 weeks of treatment, male mice at 22 months of age (equivalent to about mid-sixties in humans) fed a chow diet and received weekly administration of N-ABD094-rhArg (SEQ ID NO: 50) starting from 17-month old [CD Old (rhArg) group] in FIG. 40, showed significantly less fat mass of the perirenal (visceral) and inguinal (subcutaneous) white adipose tissue (WAT) and interscapular brown adipose tissue (BAT) in comparison to age-matched male mice fed on a chow diet and receiving vehicle [CD Old (vehicle) group] in FIG. 40, and male mice at 10 months of age (equivalent to about mid-thirties in humans) fed on a chow diet and had received vehicle injection starting from 5-month old [CD Young (vehicle) group] in FIG. 40. The mass of liver of CD Old (rhArg) group was the lowest amongst all 3 groups of mice. *P<0.05, one-way ANOVA followed by Bonferroni test. Data are expressed as mean±SEM.

FIG. 45 illustrates that C57BL/6J male mice about 25 months of age (equivalent to seventy in humans) fed an ordinary chow diet exhibited repetitive 7-day intermittent fasting cycles when administered with about 600 U N-ABD094-rhArg (SEQ ID NO: 50) (rhArg) once a week for 30 weeks. (A) Patterns of food intake of 3 groups of mice: 25-month old mice fed a chow diet and injected with rhArg [CD Very Old (rhArg) group]; 25-month old mice fed a chow diet and injected with saline (vehicle) [CD Very Old (vehicle) group]; 8-month old male mice (equivalent to early thirties in humans) fed a chow diet and injected with vehicle [CD Middle-age (vehicle) group] served as the middle-age control. (B) Change in bodyweight over the 30-week treatment period showed that very old mice exhibited rapid weight loss from 45 g to 30 g within 3 weeks after rhArg treatment, and their bodyweight was maintained relatively constant at 30 g for the rest of the treatment period with dosages of rhArg reduced to about 200 U. In contrast, the middle-aged mice treated with vehicle showed gradual increase in bodyweight, while the very old mice treated with vehicle showed gradual reduction in bodyweight over the 30-week of treatment period. (C) The survival rate CD Very old (vehicle) group gradually dropped to 20% over the course of 30-week, whereas the survival rate of CD Very old (rhArg) group was kept constant at 80% till the end of the treatment period, which demonstrated that rhArg treatment can prolong lifesp an of mice. n=6 for CD Middle-age (vehicle) group, and n=10 for CD Very old (vehicle) and CD Very Old (rhArg) groups.

FIG. 46 illustrates the enhancement of autophagic flux at Day 3 (fasting phase in the 7-day intermittent fasting cycle) in liver of DIO male mice administrated with 600 U N-ABD094-rhArg (SEQ ID NO: 50) once a week for 4 weeks. Ratio of LC3-II/LC3-I proteins was used as a marker of autophagy and was semiquantified by western blotting. Chloroquine (an autophagy inhibitor, CQ) was injected 5 hr to accumulate autophagosome in the liver before tissue collection. *P<0.05, one-way ANOVA followed by Bonferroni test. Data are expressed as mean±SEM, n=3 for each group.

FIG. 47 illustrates the liver examined by transmission electron microscopy at Day 1, Day 3, Day 5 and Day 7 of the 7-day intermittent after N-ABD094-rhArg injection. The liver of HFD-fed male mice administered with 600 U N-ABD094-rhArg (SEQ ID NO: 50) once a week for 4 weeks showed cyclic occurrence of autophagy, with Day 3 (fasting phase) demonstrated massive amount of autophagosomes. Autophagy (including the presence of lysosome, autophagosome and autolysosome) was induced to break down lipids (lipophagy).

FIG. 48 illustrates that administration of 600 U N-ABD094-rhArg (SEQ ID NO: 50) once a week to C57BL/6J male mice with pre-existing HFD-induced obesity [HFD (rhArg) group] for 12 weeks could effectively reverse hepatic steatosis, which was in line with the findings of induction of lipophagy observed by TEM. (A) Representative images of freshly dissected liver (upper panel) showing enlarged liver of pale colour in HFD-fed DIO mice treated with vehicle [HFD (vehicle) group], with extensive accumulation of lipids stained by oil Red O (lower panel) on liver sections. The liver of HFD-fed mice treated with rhArg [HFD (rhArg) group] showed rapid clearance of lipids and the size was similar to vehicle-treated control mice fed a chow diet [CD (vehicle)]. (B) Liver mass and (C) triglyceride concentrations of the 3 groups of mice were in line with the size and oil Red 0 staining results shown in A. *P<0.05, one-way ANOVA followed by Bonferroni test. Data are expressed as mean±SEM, n=5 for each group.

FIG. 49 illustrates the changes of relative p62 protein levels, detected by western blotting, at different days of a 7-day intermittent fasting cycle in the brown adipose tissue (BAT) of HFD-induced C57BL/6J DIO male mice administered with 600 U N-ABD094-rhArg (SEQ ID NO: 50) once a week for 4 weeks. The p62 protein is a receptor for cargo destined to be degraded by autophagy. The marked reduction in p62 levels from Day 1 to Day 3 indicated enhanced autophagy that coincided with the entry into the fasting phase. The increase in p62 levels from Day 5 to Day 7 implicated a decrease in autophagy, which coincided with the entry into the refeeding phase. *P<0.05, one-way ANOVA followed by Bonferroni test. Data are expressed as mean±SEM, n=4 for each group.

FIG. 50 illustrates the enhancement of autophagic flux at Day 3 (fasting phase in the 7-day intermittent fasting cycle) in the interscapular brown adipose tissue (BAT) of DIO male mice administrated with 600 U N-ABD094-rhArg (SEQ ID NO: 50) once a week for 4 weeks. Ratio of LC3II/LC3-I was used as a marker of autophagy and semiquantified by western blotting. Chloroquine (an autophagy inhibitor, CQ) were injected 5 hr to accumulate autophagosomes in the BAT before tissue collection. *P<0.05, one-way ANOVA followed by Bonferroni test. Data are expressed as mean±SEM, n=4 for [HFD (vehicle)] and [HFD (rhArg)] group without CQ; n=3 for CQ-treated group.

FIG. 51 illustrates a significant suppression of ribosomal protein S6 kinase beta-1 (p70S6K1, a downstream target of mammalian target of rapamycin mTOR) and stimulation of Unc-51 like autophagy activating kinase 1 (ULK1, an initiator of autophagy), determined by western blotting, at Day 3 (fasting phase in the 7-day intermittent fasting cycle) in the interscapular brown adipose tissue (BAT) of DIO male mice administered with 600 U N-ABD094-rhArg (SEQ ID NO: 50) once a week for 4 weeks. *P<0.05, one-way ANOVA followed by Bonferroni test. Data are expressed as mean±SEM, n=4 each group.

FIG. 52 illustrates the interscapular brown adipose tissue (iBAT) examined by transmission electron microscopy at Day 3 and Day 7 of a 7-day intermittent fasting cycle after N-ABD094-rhArg injection. (A) The BAT of HFD-fed male mice administered with 600 U N-ABD094-rhArg (SEQ ID NO: 50) [HFD (rhArg) group] once a week for 4 weeks, in comparison to that of HFD-fed mice treated with vehicle [HFD (vehicle) group] (B) showed significant reduction in the size of lipid droplets, with the presence of extensive autophagy (including the presence of lysosome, autophagosome and autolyso some) occurred at Day 3 to break down lipids (lipophagy). (C) As examined under 5000× magnification, autophagosome engulfed the lipid droplet and breaking into tiny particles. (D) At Day 7, autophagosome and autolysosome numbers had prominently reduced by Day 7, but an increase in mitochondria number was observed.

FIG. 53 illustrates that administration of 600 U N-ABD094-rhArg (SEQ ID NO: 50) once a week to C57BL/6J male mice with pre-existing HFD-induced obesity [HFD (rhArg) group] for 12 weeks could effectively reverse whitening of brown adipose tissue (BAT), with lipids stored as a large single globule (characteristic feature of white adipocytes) changing to storage of lipids in multiple small droplets (characteristic feature of brown adipocytes), which was in line with the findings of induction of lipophagy observed by TEM. (A) Representative images of fresh interscapular BAT (upper panel) showing enlarged interscapular BAT in HFD -fed DIO mice treated with vehicle [HFD (vehicle) group], with many cells exhibiting an enlarged single lipid-like globule in paraffin section stained with haematoxylin and eosin (lower panel). The iBAT of HFD-fed mice treated with rhArg [HFD (rhArg) group] showed reduction in organ size and restoration of histological appearance resembling that of vehicle-treated control mice fed a chow diet [CD (vehicle)]. (B) The mass of iBAT of the 3 groups of mice was in line with the size shown in A. *P<0.05, one-way ANOVA followed by Bonferroni test. Data are expressed as mean±SEM, n=5 for each group.

FIG. 54 illustrates that N-ABD094-rhArg (SEQ ID NO: 50) induced autophagy, using in primary culture of mouse hypothalamic neurons. (A) Increased levels of LC3II protein to β-tubulin, and (B) Decreased levels of p62 protein relative to β-tubulin, in primary culture of mouse hypothalamic neurons treated with N-ABD094-rhArg (SEQ ID NO: 50) (+) or without treatment (−) for 1, 4, 8 and 24 hrs. *P<0.05, Mann-Whitney U test. Data are expressed as mean±SEM, n=3-6.

FIG. 55 illustrates that N-ABD094-rhArg (SEQ ID NO: 50) induced activation of eIF2a/ATF4 pathway, and inactivation of mTOR pathway as demonstrated by reduced phosphorylation of P70S6K1) in primary culture of mouse hypothalamic neurons. (A) Ratio of phosphorylated eukaryotic translation initiation factor 2A (eIF2α) to total eIF2α protein levels, (B) ATF4 protein levels relative to β-tubulin, and (C) ratio of phosphorylated p70S6K1 to total p70S6K1 protein levels, in primary culture of mouse hypothalamic neurons treated with N-ABD094-rhArg (SEQ ID NO: 50) (+) or without treatment (−) for 1, 4, 8 and 24 hrs. *P<0.05, Mann-Whitney U test. Data are expressed as mean±SEM, n=4-7.

FIG. 56 illustrates that N-ABD094-rhArg (SEQ ID NO: 50) induced glycosylation of pro-opiomelanocortin POMC (active form of POMC) in primary culture of mouse hypothalamic neurons. *P<0.05, Mann-Whitney U test. Data are expressed as mean±SEM, n=4-7.

FIG. 57 illustrates a synergistic effect of combining N-ABD094-rhArg (SEQ ID NO: 50) and metformin on induction of intermittent fasting and reduced food intake. C57BL/6J male mice with pre-existing obesity, induced by feeding a high-fat diet (HFD) from 5-week old for 12 weeks, exhibited prominent repetitive 7-day intermittent fasting cycles when administered with 300 U N-ABD094-rhArg (SEQ ID NO: 50) (rhArg) in saline via intraperitoneal injection once a week in combination with 300 mg/kg metformin in water daily via gastric feeding [HFD (rhArg+Met) group] for 9 weeks. (A) Patterns of food intake of 5 groups of mice: HFD (rhArg+Met) group; HFD-fed DIO mice injected with 300 U rhArg once a week in combination with daily gastric feeding of water [HFD (rhArg) group]; HFD-fed DIO mice injected with saline once a week in combination with daily gastric feeding of 300 mg/kg metformin [HFD (Met) group]; HFD-fed mice injected with saline once a week in combination with daily gastric feeding of water [HFD (vehicle)]; mice fed a chow diet and injected with saline once a week in combination with daily gastric feeding of water [CD (vehicle) group]. (B) Average food intake on each day for 7 days after injection of rhArg or saline on Day 0. HFD (rhArg+Met) group exhibited a prominent 7-day intermittent fasting cycle, while HFD (rhArg) group also showed a 7-day pattern of decreased food intake followed by an increase of food intake, but the magnitude was less than HFD (rhArg+Met) group. (C) Total food intake per week of HFD (rhArg+Met) group was 31% less than HFD (vehicle) group. Metformin alone [HFD (Met)] also induced a significant reduction of food intake per week by 13%, whereas rhArg alone [HFD (rhArg)] did not induce significant change in total food intake per week in comparison to HFD (vehicle) group. *P<0.05, Mann-Whitney U test. Data are expressed as mean±SEM, n=5 for each group.

FIG. 58 illustrates a synergistic effect of combining 300 U N-ABD094-rhArg (SEQ ID NO: 50) once a week and 300 mg/kg metformin daily on reducing bodyweight. HFD-fed DIO mice with combined treatment of rhArg and Met [HFD (rhArg+Met) group] in FIG. 57 showed substantial reduction of bodyweight from 52 g to 35 g within 5 weeks of treatment and then their bodyweight remained relatively constant for the rest of the treatment period. In contrast, treatment of rhArg [HFD (rhArg) group] or Met [HFD (Met) group] alone could only prevent further gain in bodyweight, but did not result in weight loss, over the 9-week of treatment period.

FIG. 59 illustrates a synergistic effect of combining 300 U N-ABD094-rhArg (SEQ ID NO: 50) once a week and 300 mg/kg metformin daily on improving insulin sensitivity and glucose tolerance on HFD-fed DIO male mice [HFD (rhArg+Met) group] in FIG. 57. (A) Insulin tolerance test (ITT) and (B) glucose tolerance test (GTT) was conducted at 6 and 7 weeks after treatment respectively. Results of ITT and GTT are expressed as area under the curve (AUC).*P<0.05, one-way ANOVA followed by Bonferroni test. Data are expressed as mean±SEM.

FIG. 60 illustrates a synergistic effect of combining 300 U N-ABD094-rhArg (SEQ ID NO: 50) once a week and 300 mg/kg metformin daily on markedly reducing the mass of fat pad in perirenal (visceral) and inguinal (subcutaneous) white adipose tissue (WAT), and interscapular brown adipose tissue (BAT) in HFD-fed DIO male mice [HFD (rhArg+Met) group] in FIG. 57. *P<0.05, one-way ANOVA followed by Bonferroni test. Data are expressed as mean±SEM.

FIG. 61 illustrates a synergistic effect of combining 300 U N-ABD094-rhArg (SEQ ID NO:50) once a week and 300 mg/kg metformin daily on markedly reducing the liver mass and reversing hepatic steatosis in HFD-fed DIO male mice [HFD (rhArg+Met) group] in FIG. 57. (A) Liver mass. (B) Representative images of fresh whole liver (upper panel) and oil Red O staining of lipids in liver sections showed prominent reduction in liver mass and marked clearance of lipids in the liver of mice receiving combined therapy. (C) Triglyceride concentrations in liver were in line with oil Red O staining results. *P<0.05, one-way ANOVA followed by Bonferroni test. Data are expressed as mean±SEM.

FIG. 62 illustrates the ultrastructure of hepatocytes examined under transmission electron microscopy of HFD-fed C57BL/6J male mice with pre-existing HFD-induced obesity, which had received 3 weeks of combined therapy with 300 U N-ABD094-rhArg (SEQ ID NO: 50) once weekly and 300 mg/kg metformin daily [HFD (rhArg+Met) group], or single therapy of either 300 U N-ABD094-rhArg (SEQ ID NO: 50) once weekly [HFD (rhArg) group] or 300 mg/kg metformin daily [HFD (Met) group], or vehicle [HFD (vehicle) group]. Hepatocytes of HFD -fed mice receiving combined therapy of rhArg and metformin examined at Day 3 (fasting phase) of the 7-day intermittent fasting cycle showed marked reduction in the size of lipid droplets compared with mice treated with vehicle, and there were abundance of autophagosomes. However, the number of autophagic vesicles was greatly reduced when examined at Day 7 (refeeding phase) of the 7-day intermittent fasting cycle. Hepatocytes of HFD-fed mice receiving single therapy of rhArg at Day 3 or metformin alone showed reduction in lipid droplets size but autophagosome was rarely seen.

FIG. 63 illustrates extensive lipophagy taking place in the hepatocyte of mouse that had received combined therapy [HFD (rhArg+Met) group], which is characterized by formation of autophagosomes that sequestered portions of large lipid droplets to form the double-membrane vesicles, breaking down the lipid droplet into a smaller size. Observed by transmission electron microscopy under 5000× magnification.

FIG. 64 illustrates the occurrence of macroautophagy in the hepatocyte of mouse that had received combined therapy [HFD (rhArg+Met) group], which is characterized by a large autophagosome containing a variety of cytoplasmic components fusing with lysosomes that further formed into an autolysosome. Observed by transmission electron microscopy under 5000× magnification.

FIG. 65 illustrates that combination therapy of 300 U N-ABD094-rhArg (SEQ ID NO: 50) once weekly and 300 mg/kg metformin daily inhibited phosphorylation of mTORC1 in liver and interscapular brown adipose tissue of male mice with pre-existing HFD-induced obesity [HFD (rhArg+Met) group].The mTORC1 is a master regulator of autophagy, suppression of mTORC1 can trigger cellular autophagy. *P<0.05, one-way ANOVA followed by Bonferroni test. Data are expressed as mean±SEM, n=5 for each group.

FIG. 66 illustrates a synergistic effect of combining N-ABD094-rhArg (SEQ ID NO: 50) and all-trans retinoic acid (RA) on induction of intermittent fasting and reduced food intake. C57BL/6J male mice with pre-existing obesity, induced by feeding a high-fat diet (HFD) from 5-week old for 12 weeks, exhibited prominent repetitive 7-day intermittent fasting cycles when administered with 200 U N-ABD094-rhArg (SEQ ID NO: 50) (rhArg) in saline via intraperitoneal injection once a week in combination with 0.33 mg all-trans retinoic acid (RA) in peanut oil daily via gastric feeding [HFD (rhArg+RA) group] for 10 weeks. (A) Patterns of food intake of 5 groups of mice: HFD (rhArg+RA) group; HFD-fed DIO mice injected with 200 U rhArg once a week in combination with daily gastric feeding of peanut oil [HFD (rhArg) group]; HFD-fed DIO mice injected with saline once a week in combination with daily gastric feeding of 0.33 mg RA [HFD (RA) group]; HFD-fed mice injected with saline once a week in combination with daily gastric feeding of peanut oil [HFD (vehicle)]; mice fed a chow diet and injected with saline once a week in combination with daily gastric feeding of peanut oil [CD (vehicle) group] served as the lean control. (B) Average food intake on each day for 7 days after injection of rhArg or saline on Day 0. HFD (rhArg+RA) group exhibited a prominent 7-day intermittent fasting cycle, while HFD (rhArg) group also showed a 7-day pattern of decreased food intake followed by an increase of food intake, but the magnitude was less than HFD (rhArg+RA) group. (C) Total food intake per week of HFD (rhArg+RA) group was 41% less than HFD (vehicle) group. RA alone [HFD (RA)] or rhArg alone [HFD (rhArg) group] caused slight, but insignificant decrease in total food intake per week in comparison to HFD (vehicle) group. *P<0.05, Mann-Whitney U test. Data are expressed as mean±SEM, n=6 for each group.

FIG. 67 illustrates a synergistic effect of combining 200 U N-ABD094-rhArg (SEQ ID NO: 50) once a week and 0.33 mg RA daily on reducing bodyweight. HFD-fed DIO mice with combined treatment of rhArg and RA [HFD (rhArg+RA) group] in FIG. 66 showed substantial reduction of bodyweight from 53 g to 30 g within 7 weeks of treatment. In contrast, treatment of rhArg [HFD (rhArg) group] or RA [HFD (RA) group] alone could only prevent further gain in bodyweight, but did not result in weight loss, over the 10-week of treatment period.

FIG. 68 illustrates a synergistic effect of combining 200 U N-ABD094-rhArg (SEQ ID NO: 50) once a week and 0.33 mg RA daily on improving insulin sensitivity and glucose tolerance on HFD-fed DIO male mice [HFD (rhArg+RA) group] in FIG. 66. (A) Insulin tolerance test (ITT) and (B) glucose tolerance test (GTT) was conducted at 6 and 7 weeks after treatment respectively. Results of ITT and GTT are expressed as area under the curve (AUC).*P<0.05, one-way ANOVA followed by Bonferroni test. Data are expressed as mean±SEM.

FIG. 69 illustrates a synergistic effect of combining 200 U N-ABD094-rhArg (SEQ ID NO: 50) once a week and 0.33 mg RA daily on markedly reducing the mass of fat pad in perirenal (visceral) and inguinal (subcutaneous) white adipose tissue (WAT), and interscapular brown adipose tissue (BAT) in HFD-fed DIO male mice [HFD (rhArg+RA) group] in FIG. 66. *P<0.05, one-way ANOVA followed by Bonferroni test. Data are expressed as mean±SEM.

FIG. 70. illustrates a synergistic effect of combining 200 U N-ABD094-rhArg (SEQ ID NO:50) once a week and 0.33 mg RA daily on markedly reducing the liver mass and reversing hepatic steatosis in HFD-fed DIO male mice [HFD (rhArg+RA) group] in FIG. 66. (A) Liver mass. (B) Representative images of fresh whole liver (upper panel) and oil Red O staining of lipids in liver sections showed prominent reduction in liver mass and marked clearance of lipids in the liver of mice receiving combined therapy. (C) Triglyceride concentrations in liver were in line with oil Red O staining results. *P<0.05, one-way ANOVA followed by Bonferroni test. Data are expressed as mean±SEM.

FIG. 71 illustrates the ultrastructure of hepatocytes examined under transmission electron microscopy of HFD-fed C57BL/6J male mice with pre-existing HFD-induced obesity, which had received 3 weeks of combined therapy with 200 U N-ABD094-rhArg (SEQ ID NO: 50) once weekly and 0.33 mg RA daily [HFD (rhArg+RA) group], or single therapy of either 200 U N-ABD094-rhArg (SEQ ID NO: 50) once weekly [HFD (rhArg) group] or 0.33 mg RA daily [HFD (RA) group], or vehicle [HFD (vehicle) group]. Massive amount of large droplets accumulated in the cytoplasm of hepatocytes of HFD-fed mice treated with vehicle [HFD (vehicle) group]. Hepatocytes of HFD-fed mice receiving combined therapy of rhArg and RA examined at Day 3 (fasting phase) of the 7-day intermittent fasting cycle showed marked reduction in the size of lipid droplets, with abundance of autophagosomes. However, the number of autophagic vesicles was greatly reduced when examined at Day 7 (refeeding phase) of the 7-day intermittent fasting cycle. Hepatocytes of HFD-fed mice receiving single therapy of rhArg at Day 3 or RA alone showed reduction in lipid droplets size but autophagosome was rarely seen.

FIG. 72 illustrates extensive lipophagy taking place in the hepatocyte of mouse that had received combined therapy [HFD (rhArg+RA) group], which is characterized by formation of autophagosomes that sequestered portions of large lipid droplets to form the double-membrane vesicles, breaking down the lipid droplet into a smaller size. Observed by transmission electron microscopy under 5000× magnification.

### DETAILED DESCRIPTION

### Definition of Terms

The definitions of terms used herein are meant to incorporate the present state-of-the-art definitions recognized for each term in the field of biotechnology. Where appropriate, exemplification is provided. The definitions apply to the terms as they are used throughout this specification, unless otherwise limited in specific instances, either individually or as part of a larger group.

As used herein, the term “half-life” or “½-life” refers to the time that would be required for the concentration of an agent, e.g., a fusion protein or arginine depleting agent as described herein, to fall by half in vitro or in vivo, for example, after injection into a mammal. In certain instances, the concentration of plasma arginine, after injection, is used herein as a proxy indicator of the half-life of the agent. In such instances, the term “therapeutic duration” is used to refer to the length of time a specified dosage of the arginine depleting agent is able to maintain the plasma concentration of arginine below a specified threshold concentration that a desired therapeutic effect is observed. In certain embodiments, the threshold concentration of plasma arginine is below 50 μM, below 40 μM, below 30 μM, below 20 μM, below 10 μM, below 5 μM, below 3 μM, or at a concentration below the detection limit of conventional analytical instrumentation. For example, depletion of plasma arginine to concentrations below the detection limit of the Biochrom 30 Amino Acid Analyzer (detection limit is 3 μM) for 7 days upon injection of an arginine catabolic enzyme described herein indicates a therapeutic duration of 7 days and a half-life, e.g., on the order of around 7 days.

The term “attach” or “attached” as used herein, refers to connecting or uniting by a bond or non-bonding interaction in order to keep two or more compounds together, which encompasses either direct or indirect attachment such that for example where a first polypeptide is directly bound to a second polypeptide or other molecule, and the embodiments wherein one or more intermediate compounds (e.g., a linker), such as a polypeptide, is disposed between the first polypeptide and the second polypeptide or other molecule.

The term “protein” or “polypeptide” as used herein indicates an organic polymer composed of two or more amino acid monomers and/or analogs thereof. The term “polypeptide” includes amino acid polymers of any length including full length proteins and peptides, as well as analogs and fragments thereof. A polypeptide of three or more amino acids is also called an oligopeptide. As used herein, the term “amino acid”, “amino acidic monomer”, or “amino acid residue” refers to any of the twenty naturally occurring amino acids including synthetic amino acids with unnatural side chains and including both D and L optical isomers. The term “amino acid analog” refers to an amino acid in which one or more individual atoms have been replaced, either with a different atom, isotope, or with a different functional group but is otherwise identical to its natural amino acid analog.

As used herein, the term “unnatural amino acid” refers to any amino acid, modified amino acid, and/or amino acid analogue that is not one of the 20 common naturally occurring amino acids, seleno cysteine or pyrrolysine.

As used herein, the term “fusion protein” refers to a chimeric protein containing proteins or functional protein fragments (e.g., arginase or variants thereof) having different origins that are covalently linked, e.g., by an amide, ester, urea, carbamate, ether, and/or disulfide bond.

As used herein, the term “variant” refers to a polynucleotide or nucleic acid differing from a reference nucleic acid or polypeptide, but retaining essential properties thereof. Generally, variants are overall closely similar, and, in many regions, identical to the reference nucleic acid or polypeptide.

A variant can, for example, comprise the amino acid sequence of the parent polypeptide sequence with at least one conservative amino acid substitution. Alternatively or additionally, the variant can comprise the amino acid sequence of the parent polypeptide sequence with at least one non-conservative amino acid substitution. In this case, it is preferable for the non-conservative amino acid substitution to not interfere with or inhibit the biological activity of the functional variant. The non-conservative amino acid substitution may enhance the biological activity of the variant, such that the biological activity of the variant is increased as compared to the parent polypeptide.

The term “functional fragment” when used in reference to a polypeptide refers to any part or portion of the subject polypeptide, which part or portion retains the biological activity of the polypeptide of which it is a part (the parent polypeptide). The functional fragment can be any fragment comprising contiguous amino acids of the polypeptide of which it is a part, provided that the functional fragment still exhibits at least 40%, 50%, 60%, 70%, 80%, 90%, 95%, or 99% or has substantially the same or even higher biological activity of the parent polypeptide. In reference to the parent polypeptide, the functional fragment can comprise, for instance, about 10%, 20%, 25%, 30%, 40%, 50%, 60%, 70%, 80%, 90%, 95%, 98% or more, of the parent polypeptide.

The functional fragment can comprise additional amino acids at the amino or carboxy terminus, or at both termini, e.g., amino acids not found in the amino acid sequence of the parent polypeptide.

Amino acid substitutions of the described polypeptides can be conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a beta-branched side-chain substituted for another amino acid with a beta-branched side-chain (e.g., Ile, Thr, and Val), an amino acid with an aromatic side-chain substituted for another amino acid with an aromatic side chain (e.g., His, Phe, Trp, and Tyr), etc.

The terms “percentage homology” and “percentage sequence identity”, when used in reference to a polypeptide or polynucleotide sequence, are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Homology is evaluated using any of the variety of sequence comparison algorithms and programs known in the art. Such algorithms and programs include, but are by no means limited to, TBLASTN, BLASTP, FASTA, TFASTA, and CLUSTALW [Pearson and Lipman, 1988, Proc. Natl. Acad. Sci. USA 85(8):2444-2448; Altschul et al., 1990, J. Mol. Biol. 215(3):403-410; Thompson et al., 1994, Nucleic Acids Res. 22(2):4673-4680; Higgins et al. 1996, Methods Enzymol. 266:383-402; Altschul et al., 1990, J. Mol. Biol. 215(3):403-410; Altschul et al., 1993, Nature Genetics 3:266-272]. In certain embodiments, protein and nucleic acid sequence homologies are evaluated using the Basic Local Alignment Search Tool (“BLAST”) which is well known in the art (see, e.g., Karlin and Altschul, 1990, Proc. Natl. Acad. Sci. USA 87:2267-2268; Altschul et al., 1990, J. Mol. Biol. 215:403-410; Altschul et al., 1993, Nature Genetics 3:266-272; Altschul et al., 1997, Nuc. Acids Res. 25:3389-3402).

As used herein, the terms “treat”, “treating”, “treatment”, and the like refer to reducing or ameliorating a disorder/disease and/or symptoms associated therewith. It will be appreciated, although not precluded, treating a disorder or condition does not require that the disorder, condition, or symptoms associated therewith be completely eliminated. In certain embodiments, treatment includes prevention of a disorder or condition, and/or symptoms associated therewith. The term “prevention” or “prevent” as used herein refers to any action that inhibits or at least delays the development of a disorder, condition, or symptoms associated therewith. Prevention can include primary, secondary and tertiary prevention levels, wherein: a) primary prevention avoids the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

As used herein, the terms “co-administration” and “co-administering” refer to both concurrent administration (administration of two or more therapeutic agents at the same time) and time varied administration (administration of one or more therapeutic agents at a time different from that of the administration of an additional therapeutic agent or agents). In certain embodiments, the therapeutic agents are present in the patient to some extent at the same time.

As used herein the term “catabolism” or “catabolic” refers to the chemical reaction of a molecule into other, e.g., smaller, molecules. For example, arginine catabolic enzymes refer to any enzyme capable of reacting with arginine thereby transforming it into other molecules, such as ornithine, citrulline, and agmatine.

As used herein, the term “subject” refers to any animal (e.g., a mammal), including, but not limited to, humans, non-human primates, canines, felines, and rodents.

Provided herein is a method of inducing intermittent fasting, modulating autophagy, or inducing intermittent fasting and modulating autophagy in a subject in need thereof comprising the step of administering a therapeutically effective amount of an arginine depleting agent to the subject.

In recent years, a great deal of research has been directed to studying the effects of intermittent fasting and/or modulating autophagy, and the numerous health benefits that can result from inducting intermittent fasting and/or modulating autophagy. For example, it has been found intermittent fasting and/or modulating autophagy can improve longevity of a subject (Adv Nutr. 2019 Nov. 1; 10(Supplement_4):S340-S350. doi: 10.1093/advances/nmz079; Aging Cell. 2019 February; 18(1):e12843. doi: 10.1111/acel.12843. Epub 2018 Oct. 17.); treat cardiovascular disease (Circ Res. 2019 Mar. 15; 124(6):952-965. doi: 10.1161/CIRCRESAHA.118.313352.); treat inflammatory bowel disease (Cell Rep. 2019 Mar. 5; 26(10):2704-2719.e6. doi: 10.1016/j.celrep.2019.02.019.); treat diabetes (Cell. 2017 Feb. 23; 168(5):775-788.e12. doi: 10.1016/j.cell.2017.01.040.); treating aging, cancer, and cardiovascular disease (Sci Transl Med. 2017 Feb. 15; 9(377). pii: eaai8700. doi: 10.1126/scitranslmed.aai8700); treat autoimmune diseases (Mol Cell Endocrinol. 2017 Nov. 5; 455:4-12. doi: 10.1016/j.mce.2017.01.042. Epub 2017 Jan. 28.); treatment of age-related disorders including diabetes, cardiovascular disease, cancers and neurological disorders such as Alzheimer's disease, Parkinson's disease and stroke (Ageing Res Rev. 2017 October; 39:46-58. doi: 10.1016/j.arr.2016.10.005. Epub 2016 Oct. 31.); treating autoimmunity and multiple sclerosis symptoms (Cell Rep. 2016 Jun. 7; 15(10):2136-2146. doi: 10.1016/j.celrep.2016.05.009. Epub 2016 May 26.); improving cognition, performance, health span, lowered visceral fat, reduced cancer incidence and skin lesions, rejuvenated the immune system, and retarded bone mineral density loss (Cell Metab. 2015 Jul. 7; 22(1):86 -99. doi: 10.1016/j.cmet.2015.05.012. Epub 2015 Jun. 18.); and reduce obesity, hypertension, asthma, and rheumatoid arthritis (Cell Metab. 2014 Feb. 4; 19(2):181-92. doi: 10.1016/j.cmet.2013.12.008. Epub 2014 Jan. 16), all of which are hereby incorporated by reference.)

Research has also demonstrated that intermittent fasting can be used in the treatment of cancer (Nat Rev Cancer. 2018 November; 18(11):707-719. doi: 10.1038/s41568-018-0061-0; Recent Results Cancer Res. 2016; 207:241-66. doi: 10.1007/978-3-319-42118-6_12; Cancer Cell. 2016 Jul. 11; 30(1):136-146. doi: 10.1016/j.ccell.2016.06.005; Mol Cell Oncol. 2015 Dec. 10; 3(3):e1117701. doi: 10.1080/23723556.2015.1117701. eCollection 2016 May; Oncotarget. 2015 May 20; 6(14):11820-32; PLoS One. 2012; 7(9):e44603. doi: 10.1371/journal.pone.0044603. Epub 2012 Sep. 11; Drug Resist Updat. 2012 February-April; 15(1-2):114-22. doi: 10.1016/j.drup.2012.01.004. Epub 2012 Mar. 4; Sci Transl Med. 2012 Mar. 7; 4(124):124ra27. doi: 10.1126/scitranslmed.3003293. Epub 2012 Feb. 8; Oncogene. 2011 Jul. 28; 30(30):3305-16. doi: 10.1038/onc.2011.91. Epub 2011 Apr. 25; and Cell Cycle. 2010 Nov. 15; 9(22):4474-6. Epub 2010 Nov. 15), all of which are hereby incorporated by reference.

In certain embodiments, the arginine depleting agents described herein can be used in the treatment of any disease or health condition for which inducing intermittent fasting and/or modulating autophagy has a beneficial effect. In certain embodiments, the disease or health condition for which inducing intermittent fasting and/or modulating autophagy has a beneficial effect is any one or more of the diseases or health conditions described above.

In certain embodiments, provided herein is a method comprising the step of administering a therapeutically effective arginine depleting agent to induce intermittent fasting and/or modulate autophagy to improve longevity and/or alleviates a symptom of aging or preventing age related diseases.

In certain embodiments, provided herein is a method comprising the step of administering a therapeutically effective arginine depleting agent to induce intermittent fasting and/or modulate autophagy to promote clearance of protein aggregates, and prevent and/or treat neurodegenerative diseases, such as Alzheimer's.

In certain embodiments, provided herein is a method comprising the step of administering a therapeutically effective arginine depleting agent to induce intermittent fasting and/or modulate autophagy to treat inflammation and related diseases including rheumatoid arthritis.

In certain embodiments, provided herein is a method comprising the step of administering a therapeutically effective arginine depleting agent to induce intermittent fasting and/or modulate autophagy to treat diseases associated with deficits in autophagy.

In certain embodiments, provided herein is a method comprising the step of administering a therapeutically effective arginine depleting agent to induce intermittent fasting and/or modulate autophagy to promote clearance of intracellular pathogens to treat bacterial and viral infections.

The arginine depleting agent can be any arginine depleting agent known in the art that is capable of reducing plasma and/or cellular levels of arginine in a subject. The arginine depleting agent can be a small molecule or protein.

The protein can be a fusion protein and/or a chemically modified protein, such as a PEGylated protein. Exemplary proteins include those that are capable of catalyzing the catabolism of arginine to other products, such as proteins having arginase, arginine deiminase, arginine decarboxylase, or arginine 2 monooxygenase activity.

The arginase can be any arginase known in the art, such as those produced by bacteria, fungi, fish, human, bovine, swine, rabbit, rodent, primate, sheep and goat. For example, Bacillus caldovelox arginase, Thermus thermophilus arginase, Capra hircus arginase I, Heterocephalus glaber arginase I, Bos taurus arginase I, Sus scrofa arginase I, Plecoglossus altivelis arginase I, Salmo salar arginase I, Oncorhynchus mykiss arginase I, Osmerus mordax arginase I, Hyriopsis cumingii arginase I, Rattus norvegicus arginase I, Mus musculus arginase I, Homo sapiens (human) arginase I, Pan troglodytes arginase I, Oryctolagus cuniculus arginase I, Rattus norvegicus arginase II, Mus musculus arginase II, Homo sapiens (human) arginase II, Bostaurus arginase II, Heterocephalus glaber arginase II, Pan troglodytes arginase II, Oryctolagus cuniculus arginase II, Delftia arginase, Bacillus coagulans arginase, Hoeflea phototrophica arginase and Roseiflexus castenholzii arginase. Other examples include arginases from Bacillus methanolicus, Bacillus sp. NRRL B-14911, Planococcus donghaensis, Paenibacillus dendritiformis, Desmospora sp., Methylobacter tundripaludum, Stenotrophomonas sp., Microbacterium laevaniformans, Porphyromonas uenonis, Agrobacterium sp., Octadecabacter arcticus, Agrobacterium tumefaciens, Anoxybacillus flavithermus, Bacillus pumilus, Geobacillus thermoglucosidasius, Geobacillus thermoglucosidans, Brevibacillus laterosporus, Desulfotomaculum ruminis, Geobacillus kaustophilus, Geobacillus thermoleovorans, Geobacillus thermodenitrificans, Staphylococcus aureus, Halophilic archaeon DL31, Halopigerxanaduensis, Natrialba magadii, Plasmodium falciparum, Helicobacter pylori, and the like.

The arginine deiminase can be any arginine deiminase known in the art, such as those produced from Mycoplasma, Lactococcus, Pseudomonas, Steptococcus, Escherichia, Mycobacterium or Bacillus microorganisms. Exemplary arginine deiminase include, but are not limited, to those produced by Mycoplasma hominis, Mycoplasma arginini, Mycoplasma arthritidis, Clostridium perfringens, Bacillus licheniformis, Borrelia burgdorferi, Borrelia afzellii, Enterococcus faecalis, Lactococcus lactis, Bacillus cereus, Streptococcus pyogenes, Steptococcus pneumoniae, Lactobacillus sake, Giardia intestinalis, Mycobacterium tuberculosis, Pseudomonas plecoglossicida, Pseudomonas putida, Pseudomonas aeruginosa, and the like.

The arginine decarboxylase can be any arginine decarboxylase known in the art, such as those produced by Escherichia coli., Salmonella typhimurium, Chlamydophila pneumoniae, Methanocaldococcus jannaschii, Paramecium bursaria Chlorella virus 1, Vibrio vulnificus YJ016, Campylobacter jejuni subsp., Trypanosoma cruzi, Sulfolobus solfataricus, Bacillus licheniformis, Bacillus cereus, Carica papaya, Nicotianatobacum, Glycine max, Lotus coniculata, Vibrio vulnificus, Vibrio cholerae, Mus musculus, Thermotoga, Rattus norvegicus, Homo sapiens, Bos taurus, Susscrofa, Thermus thermophiles, Thermus parvatiensis, Thermus aquaticus, Thermus thermophilus, Thermus islandicus, Arabidopsis thaliana, Avena sativa, and the like.

The arginine 2-monooxygenase can be any arginine 2-monooxygenase known in the art, such as those produced from Arthrobacter globiformis IFO 12137, Arthrobacter simplex IFO 12069, Brevibacterium helvolum IFO 12073, Helicobacter cinaedi CCUG 18818, Streptomyces griseus, and the like.

The arginine decarboxylase, arginine deiminase, arginine 2-mono-oxygenase, and arginase can be the full protein or a functional fragment and/or variant thereof. The arginine decarboxylase, arginine deiminase, arginine 2-mono-oxygenase, and arginase can be modified to improve their pharmacokinetic properties, such as by fusion of the protein or functional fragment and/or variant thereof with human serum albumin, an albumin binding domain, an Fc region of immunoglobulin, a PEG group, or a combination thereof.

The arginine catabolic enzymes described herein can be engineered to include specific sites on the enzyme where PEG can be selectively attached. The selected PEGylation sites are preferably located at a site removed from the active site of the enzyme, and generally exposed to solvent to allow reaction with PEGylation reagents.

For example, Cys⁴⁵-human arginase I (HAI) and Cys¹⁶¹-Bacillus caldovelox arginase (BCA) can be produced to react with thiol-specific PEG molecules. Conjugation between the single, free cysteine residue of the modified arginase and a maleimide group (MAL) attached to a PEG compound can result in a covalent bond between the PEG compound and the free cysteine of the modified arginase. SEQ ID NOs: 102 and 104 include mutant (C168S/C303S) designed for Cys⁴⁵ site-directed PEGylation and thus can optionally be PEGylated. SEQ ID NO: 89 also includes mutant (S161C) designed for Cys¹⁶¹ site-directed PEGylation and thus can optionally be PEGylated.

In certain embodiments the arginase can comprise SEQ ID NO: 101, SEQ ID NO: 102, SEQ ID NO: 103, or SEQ ID NO: 104, wherein SEQ ID NO: 102 and SEQ ID NO: 104 optionally comprise a polyethylene glycol group (PEG).

Any PEGylation reagent known in the art can be used to covalently attach PEG to the arginine catabolic enzymes described herein. Exemplary PEGylation reagents include, but are not limited to mPEG-ALD (methoxypolyethylene glycol-propionaldehyde); mPEG-MAL (methoxypolyethylene glycol-maleimide); mPEG-NHS (methoxypolyethylene glycol-N-hydroxy-succinimide); mPEG-SPA (methoxypolyethylene glycol-succinimidyl propionate); and mPEG-CN (methoxypolyethylene glycol-cyanuric chloride).

The PEG group can have a molecular weight of about 5,000 to about 20,000 amu, about 5,000 to about 15,000 amu, about 5,000 to about 12,000 amu, about 7,000 to about 12,000 amu, or about 7,000 to about 10,000 amu. In certain embodiments, the PEG group has a molecular weight of about 2,000 amu to 10,000 amu. In certain embodiments, the PEG group is PEG4,000, PEG5,000, PEG6,000, or PEG7,000.

The PEG group can be covalently attached directly to the arginase or via a linker. In certain embodiments, the arginase is covalently attached via a propionic acid linker to PEG. In other embodiments, the arginase is covalently attached via a C2-C10, C2-C9, C2-C8, C2-C7, C2-C6, C2-C5, or C2-C4 straight or branched chain carboxylic acid linker to PEG.

In certain embodiments, the fusion proteins provided herein comprise an arginase polypeptide. The arginase polypeptide can be derived from an arginase protein expressed by any organism that expresses arginase. Exemplary arginases include those that are produced by bacteria, such as bacilli, agrobacteria, cyanobacteria, and mycobacteria, and mammals, such as bovine, porcine, sheep, goat, rodents and humans. When the arginase polypeptide is derived from human arginase, it can be arginase type 1 (ARG1) or arginase type 2 (ARG2).

The arginase polypeptide can comprise a full length arginase polypeptide or a functional fragment and/or variant thereof.

Arginase is a manganese-containing enzyme. As demonstrated in the Example 32, when one or more of the manganese ions present in the fusion proteins described herein are replaced with one or more divalent cationic metal, such as Co²⁺ or Ni²⁺, the catalytic activity of the fusion protein can increase. Accordingly, in certain embodiments, the fusion proteins described herein comprise one or more divalent metals, other than manganese, such as Co²⁺ or Ni²⁺. In certain embodiments, the fusion protein comprises one or more metals selected from Co²⁺ and Ni²⁺. In certain embodiments, the fusion protein comprises two Co²⁺ ions or two Ni²⁺ ions. In other embodiments, the fusion protein comprises two Mn²⁺ ions.

In certain embodiments, the arginase polypeptide is wild type human ARG1. In certain embodiments, the arginase polypeptide comprises a sequence with at least 95% sequence homology to SEQ ID NO: 69. For example, the arginase polypeptide can comprise at a polypeptide sequence with at least 96%, 97%, 98%, 99%, 99.1%, 99.4% or 99.7% homology to SEQ ID NO: 69. In certain embodiments, the sequence of the arginase polypeptide can differ from SEQ ID NO: 69 by 1, 2, 3, 4, 5, 6, 7, 8, 9, 10, 15, 20, 25, or 30 amino acid modifications (e.g., insertion, substitution, deletion, etc.). In certain embodiments, the arginase polypeptide comprises a polypeptide with conservative amino acid replacements, non-conservative amino acid replacements, or a combination thereof.

In certain embodiments, the arginase polypeptide is Bacillus caldovelox arginase (BCA). In certain embodiments, the arginase polypeptide comprises a sequence with at least 95% sequence homology to SEQ ID NO: 70. For example, the arginase polypeptide can comprise at a polypeptide sequence with at least 96%, 97%, 98%, 99%, 99.3%, or 99.7% homology to SEQ ID NO: 70. In certain embodiments, the sequence of the arginase polypeptide can differ from SEQ ID NO: 70 by 1, 2, 3, 4, 5, 6, 7, 8, 9, 10, 15, 20, 25, or 30 amino acid modifications (e.g., insertion, substitution, deletion, etc.). In certain embodiments, the arginase polypeptide comprises a polypeptide with conservative amino acid replacements, non-conservative amino acid replacements, or a combination thereof.

In certain embodiments, the arginase polypeptide is BCA, wherein serine 161 is replaced by a cysteine as presented in SEQ ID NO: 71 and SEQ ID NO: 72. The substitution of serine with a cysteine allows for the sited directed incorporation of chemical moieties that can further improve the properties of the fusion protein. For example, the side chain of cysteine 161 can be reacted with an appropriately activated PEG moiety thereby forming a PEGylated arginase, which can be incorporated into the resulting fusion protein. PEGylation of the fusion protein has the ability to further enhance the retention time of the fusion proteins described herein by further protecting them against various degrading mechanisms active inside a tissue or cell, which consequently improves their therapeutic potential. Accordingly, in certain embodiments the arginase polypeptide comprises a sequence with at least 95% sequence homology to SEQ ID NO: 71 or SEQ ID NO: 72. For example, the arginase polypeptide can comprise at a polypeptide sequence with at least 96%, 97%, 98%, 99%, 99.3%, or 99.7% homology to SEQ ID NO: 71 or SEQ ID NO: 72. In certain embodiments, the sequence of the arginase polypeptide can differ from SEQ ID NO: 71 or SEQ ID NO: 72 by 1, 2, 3, 4, 5, 6, 7, 8, 9, 10, 15, 20, 25, or 30 amino acid modifications (e.g., insertion, substitution, deletion, etc.). In certain embodiments, the arginase polypeptide comprises a polypeptide with conservative amino acid replacements, non-conservative amino acid replacements, or a combination thereof.

In instances where the fusion proteins described herein are PEGylated, the PEG group can have a molecular weight of about 5,000 to about 20,000 amu, about 5,000 to about 15,000 amu, about 5,000 to about 12,000 amu, about 7,000 to about 12,000 amu, or about 7,000 to about 10,000 amu. In certain embodiments, the PEG group has a molecular weight of about 4,000 amu to 10,000 amu. In certain embodiments, the PEG group is PEG4,000 or PEG7,000.

The PEG group can be covalently attached directly to the fusion protein or via a linker. PEG group can be covalently attached to the fusion protein by reaction of a cysteine or lysine side chain present on the protein with a PEGylation reagent. Alternatively, the PEG group can be covalently attached to the N-terminal amine of the protein.

In certain embodiments, the fusion protein is covalently attached via a propionic acid linker to PEG. In other embodiments, the fusion protein is covalently attached via a C2-C10, C2-C9, C2-C8, C2-C7, C2-C6, C2-C5, or C2-C4 straight or branched chain carboxylic acid linker to PEG. In certain embodiments, the PEG group is attached to the arginase polypeptide.

The fusion proteins described herein also comprise an albumin binding domain (ABD) polypeptide. A number of studies have demonstrated the potential of albumin binding to achieve longer half-lives of therapeutic proteins. However, the design of fusion proteins including ABD polypeptides can be challenging, because fusion of the ABD polypeptide to the protein therapeutic has the potential to affect both the efficacy of the protein therapeutic, the binding affinity of the ABD polypeptide, and the solubility of the fusion protein. Accordingly, the selection of the ABD polypeptide, its site of attachment, and the construction of any necessary linkers is not a straight forward process and often times requires trial and error in order to arrive at a fusion property with the desired properties. For example, the therapeutic duration (and half-life) of N-ABD094-rhArg (SEQ ID NO: 50) is unexpectedly much longer than BHA and BAH. While all of BHA, BAH, and N-ABD094-rhArg exhibit surprisingly high therapeutic duration (and half-life), it could not have been predicted that N-ABD094-rhArg would have such long therapeutic duration (and half-life).

Longer action of a drug is generally desirable. The linker type, length, flexibility, and fusion of the bioactive peptide or protein to the C or N terminus of the half-life-extension module, can have profound effects on the activity of a fusion protein. In the present disclosure, various arginases were fused to ABD molecules via a suitable linker so that both the arginase enzymatic activity and the albumin binding ability of ABD can be retained. Good stability and solubility are also essential. This is very difficult and challenging to achieve. Unlike the common HSA or Fc fusions, very little is known about the ABD fusions. The present disclosure provides examples of the linker design that can be used to generate functional arginase-ABD fusions. The activity of the engineered arginase fusion proteins (N-ABD094-rhArg, N-ABD-rhArg, etc) have been optimized in the present disclosure either through linker engineering or the position of the bioactive protein (arginase) with respect to the half-life-extension module (ABD), or both.

In many cases, protein engineering can be used to overcome the loss of activity. In one example, linker engineering was used to regain activity lost upon fusion of IFN-α2b to HSA [Prot Exp Purif. 2008; 61:73-7]. A direct fusion of IFN-α2b to HSA resulted in an unstable protein with very little biological activity. In a fusion format, the effects of various linkers on the activity of IFN-α2b were tested. Peptide linkers are known to have an influence on the expression, activity, and pharmacokinetics of fusion proteins [Adv Drug Deliv Rev. 2013; 65:1357-69]. The two major types of peptide linkers are: (i) flexible linkers (e.g., (G4S)n, where n=1-4); (ii) rigid linkers such as the α-helical linker [A(EAAAK)nA]x (where n=2-4 and x=1 or 2), and XPn (where X is either A, K or E). For flexible linkers, one advantage is that the flexibility may be required to obtain proper orientation of the bioactive portion of the molecule with respect to its cognate receptor. However, flexible linkers do not give a lot of space between the fusion partner and the bioactive protein. On the other hand, rigid linkers provide more space but lack the flexibility. In the case of the IFN-α2b-HSA fusion protein, the flexible linker resulted in approximately 39% activity as compared with that of native IFN-α2b, whereas the rigid XP linker and the α-helical linker resulted in 68 and 115% of the activity of native IFN-α2b, respectively [Prot Exp Purif. 2008; 61:73-7].

Certain linkers can have a negative impact on fusion protein properties. For example, granulocyte colony-stimulating factor (G-CSF) was fused to transferrin (Tf). The use of a short leucine-glutamate (LE) linker resulted in only approximately 10% of the activity of native G-CSF. Insertion of a (G4S)3 or α-helical [A(EAAAK)nA]m (n=2-4, m=1 or 2) linker significantly increased the activity of the fusion proteins over that of G-CSF-LE-Tf. The fusion protein constructed with the linker (A(EAAAK)4ALEA-(EAAAK)4A) resulted in biological activity near to that of native G-CSF [Pharm Res. 2006; 23:2116-21]. In another example, while the C-terminus of the Fc moiety may be directly linked to the N-terminus of the IFN-β moiety via a peptide bond, Gillies et al. [U.S. Pat. No. 7,670,595 B2] additionally connects the Fc moiety and the IFN-β moiety via a linker peptide. The linker peptide is located between the C-terminus of the Fc moiety and the N-terminus of the mature IFN-β moiety. In this case, the linker peptide is preferably composed of serine and glycine residues such as the amino acid sequence G4SG4SG3SG. All these findings demonstrate the importance of testing linker technology for the success of fusion protein research and development programs.

Using a different approach, the importance of fusion position for activity has been demonstrated [Curr Pharmaceut Biotechnol. 2014; 15:856-63]. The brain natriuretic peptide (BNP) was fused to either the N or C terminus of HSA in various formats. The results showed that BNP-HSA, BNP2-HSA (two copies of BNP), and BNP4-HSA, all fused to the N terminus of HSA, were not active. However, HSA-BNP2, fused to the C-terminus of HSA, was as active as native BNP and long-lasting.

These examples demonstrate the importance of having a significant effort in lead optimization of fusion proteins to optimize the activity either through linker engineering, the position of the bioactive protein or peptide with respect to the half-life-extension module, or both. Importantly, the present invention used a new ABD fusion approach to join an ABD and an arginase together so that arginase activity can be retained. Stable and soluble arginase-ABD fusion molecules that can bind to FcRn in a pH-dependent manner were successfully generated, allowing for efficient endosomal recycling. For example, the present disclosure surprisingly found that for the rhArg fused to ABD, the terminal half-life in circulation of the protein dramatically increased from a few minutes to 4 days in mice. Taken together, a variety of different approaches are available for fine-tuning the pharmacokinetic properties of arginases, ensuring appropriate residence time in circulation for different disease conditions. Another key issue in the development of any therapeutic protein is to minimize immunogenicity to avoid adverse effects. Thus, the observed low immunogenicity of human arginase and the successful deimmunization of ABD (e.g. ABD094), which was also used in the present disclosure as gene fusion partner to extend the in vivo half-life, are important.

The albumin binding protein is a three-helical protein domain found in various surface proteins expressed by Gram positive bacteria. The albumin binding protein derived from Streptococcal protein G has 214 amino acids and contains three albumin binding domains (ABD1-3), which are used to bind to human serum albumin and evade the immune system of a host. ABD3 corresponds to a 46 amino acid sequence, which has been demonstrated to bind to human serum albumin and has been the subject of a number of studies and affinity maturation for human serum albumin to develop ABD polypeptides with differing properties, such as binding affinity and binding selectivity. Such studies have generated a substantial number of ABD polypeptides with widely varying properties.

Albumin binding proteins are found in other bacteria. For example, naturally occurring albumin binding proteins include certain surface proteins from Gram positive bacteria, such as Streptococcal M proteins (e.g. M1/Emm1, M3 Emm3, M12/Emm12, EmmL55/Emm55, Emm49/EmmL49 and Protein H), streptococcal proteins G, MAG and ZAG, and PPL and PAB from certain strains of Finegoldia magna.

In certain embodiments, the fusion proteins described herein comprise an ABD polypeptide that is derived from a Streptococcal protein G albumin binding domain. In certain embodiments, the ABD polypeptide is the full Streptococcal protein G albumin binding domain 3 or a functional fragment and/or variant thereof.

In certain embodiments, the fusion protein comprises an ABD polypeptide comprising a polypeptide sequence having at least 93% sequence homology with SEQ ID NO: 66. For example, the ABD polypeptide can comprise at a polypeptide sequence with at least 94%, 96%, or 98% homology to SEQ ID NO: 66. In certain embodiments, the sequence of the ABD polypeptide can differ from SEQ ID NO: 66 by 1, 2, 3, 4, 5, 6, 7, 8, 9, or 10 amino acid modifications (e.g., insertion, substitution, deletion, etc.). In certain embodiments, the ABD polypeptide comprises a polypeptide with conservative amino acid replacements, non-conservative amino acid replacements, or a combination thereof.

In certain embodiments, the fusion protein comprises an ABD polypeptide comprising a polypeptide sequence having at least 93% sequence homology with SEQ ID NO: 67. For example, the ABD polypeptide can comprise at a polypeptide sequence with at least 94%, 96%, or 98% homology to SEQ ID NO: 67. In certain embodiments, the sequence of the ABD polypeptide can differ from SEQ ID NO: 67 by 1, 2, 3, 4, 5, 6, 7, 8, 9, or 10 amino acid modifications (e.g., insertion, substitution, deletion, etc.). In certain embodiments, the ABD polypeptide comprises a polypeptide with conservative amino acid replacements, non-conservative amino acid replacements, or a combination thereof.

In certain embodiments, the fusion protein comprises an ABD polypeptide comprising a polypeptide sequence having at least 93% sequence homology with SEQ ID NO: 68. For example, the ABD polypeptide can comprise at a polypeptide sequence with at least 93%, 95%, or 97% homology to SEQ ID NO: 68. In certain embodiments, the sequence of the ABD polypeptide can differ from SEQ ID NO: 68 by 1, 2, 3, 4, 5, 6, 7, 8, 9, or 10 amino acid modifications (e.g., insertion, substitution, deletion, etc.). In certain embodiments, the ABD polypeptide comprises a polypeptide with conservative amino acid replacements, non-conservative amino acid replacements, or a combination thereof.

The relative position of the ABD polypeptide and arginase polypeptide can vary. For example, the ABD polypeptide can precede the arginase polypeptide (e.g., the arginase polypeptide can be attached either directly or indirectly from the C-terminal of the ABD polypeptide) or the arginase polypeptide can precede the ABD polypeptide (e.g., the ABD polypeptide can be attached either directly or indirectly from the C-terminal of the arginase polypeptide).

In certain embodiments, the fusion protein can include one or more arginase polypeptides and/or one or more ABD polypeptides. For example, the fusion protein can have the general structure ABD-rhArg-ABD, ABD094-rhArg-ABD094, ABD-BCA-ABD, ABD094-BCA-ABD094, rhArg-ABD-rhArg, rhArg-ABD094-rhArg, BCA-ABD-BCA, or BCA-ABD094-BCA.

The ABD polypeptide and the arginase polypeptide can be attached by direct covalent attachment or indirectly attached via a peptide linker.

The peptide linker or linker is a polypeptide typically ranging from about 1, 2, 3, 4, 5, 6, 7, 8, 9, 10, 11, 12, 13, 14, 15, 16, 17, 18, 19, 20 or more amino acids in length, which is designed to facilitate the functional connection of the ABD polypeptide and arginase polypeptide into a linked fusion protein. The term functional connection denotes a connection that facilitates proper folding of the polypeptides into a three dimensional structure that allows the linked fusion protein to exhibit some or all of the functional aspects or biological activities of the protein(s) from which its polypeptide constituents are derived.

The polypeptide linker can be disposed between the N-terminal of the ABD polypeptide and the C-terminal of the arginase polypeptide or alternatively disposed between the N-terminal of the arginase polypeptide and the C-terminal of the ABD polypeptide.

The peptide linker can comprise naturally occurring amino acids, unnatural amino acids, and combinations thereof.

In certain embodiments, the peptide linker can comprise glycine, serine, asparagine, or a combination thereof. Exemplary peptide linkers include linkers containing poly-glycine, (GS)ₙ, and (GGS)ₙ, wherein n is 1-30 Additional exemplary peptide linkers include flexible linkers (e.g., (G₄S)ₙ, wherein n=1-4), or rigid linkers (e.g., the alpha-helical linker [A(EAAAK)ₙA]ₓ, wherein n=2-4 and x=1 or 2; and XPn, wherein X is either A, K or E and n=1-10). In certain embodiments, the peptide linker is (A(EAAAK)₄ALEA-(EAAAK)₄A) (SEQ ID NO: 105), G₄SG₄SG₃SG (SEQ ID NO: 106), GS(N)ₙGSG, where n=1-10, and GS(Q)ₙGSG, where n=1-10, and the like. In certain embodiments, the peptide linker comprises a polypeptide sequence having at least 90% sequence homology with SEQ ID NO: 73. For example, peptide linker can comprise a polypeptide having at least a 91%, 92%, 93%, 94%, 95%, 96%, 97%, 98%, or 99% sequence homology or is identical to SEQ ID NO: 73.

In certain embodiments, the peptide linker can comprise glycine, serine, asparagine, or a combination thereof. In certain embodiments, the peptide linker comprises a polypeptide sequence having at least 90% sequence homology with SEQ ID NO: 74. For example, peptide linker can comprise a polypeptide having at least a 91%, 92%, 93%, 94%, 95%, 96%, 97%, 98%, or 99% sequence homology or is identical to SEQ ID NO: 74.

Purification tags can be used to improve the ease of purifying the fusion protein, such as by affinity chromatography. A well-known purification tag is the hexa-histidine (6× His) tag, which is a sequence of six histidine residues. Thus, in certain embodiments, the fusion protein further comprises a poly-histidine comprising 4-8 histidine amino acids, e.g., the 6× His tag. The poly-histidine can be present at the C-terminal of the fusion protein, the N-terminal of the fusion protein, or disposed in between ABD polypeptide and the arginase polypeptide.

When the poly-histidine is disposed in between the ABD polypeptide and the arginase polypeptide it can act as a peptide linker or can be included in addition to the peptide linker. For example, the fusion protein of SEQ ID NO 75 includes a six histidine polypeptide linker at position 300-305, which serves to link the ABD polypeptide and the arginase polypeptide and advantageously can be used to purify the fusion protein by affinity chromatography.

The poly-histidine tag can be optionally removed after purification is complete using techniques generally known in the art. For example, exopeptidases can be used to remove N-terminal poly-histidine tags (e.g., Qiagen TAGZyme) and C-terminal poly-histidine tags can be preceded by a suitable amino acid sequence that facilitates a removal of the poly-histidine-tag using endopeptidases. Thus, fusion proteins excluding the N-terminal and/or C-terminal poly-histidine tag are encompassed within the scope of this disclosure.

In certain embodiments, the ABD polypeptide comprises a polypeptide sequence having at least 93% sequence homology with SEQ ID NO: 66, SEQ ID NO: 67, or SEQ ID NO: 68 and the arginase polypeptide comprises a polypeptide sequence having at least 95% homology with SEQ ID NO: 69, SEQ ID NO: 70, SEQ ID NO: 71, or SEQ ID NO: 72. For example, the ABD polypeptide comprises a polypeptide sequence having at least 94%, 95%, 96%, 97%, 98%, or 99% sequence homology or is identical with SEQ ID NO: 66, SEQ ID NO: 67, or SEQ ID NO: 68 and the arginase polypeptide comprises a polypeptide sequence having at least 96%, 97%, 98%, or 99% sequence homology or is identical with SEQ ID NO: 69, SEQ ID NO: 70, SEQ ID NO: 71, or SEQ ID NO: 72.

In certain embodiments, the ABD polypeptide comprises a polypeptide sequence having at least 93%, 94%, 95%, 96%, 97%, 98%, or 99% sequence homology or is identical with SEQ ID NO: 66 and the arginase polypeptide comprises a polypeptide sequence having at least 95%, 96%, 97%, 98%, or 99% sequence homology or is identical with SEQ ID NO: 69.

In certain embodiments, the fusion protein further comprises a peptide linker comprising a polypeptide sequence having at least 90%, 91%, 92%, 93%, 94%, 95% , 96%, 97%, 98%, or 99% sequence homology or identical with SEQ ID NO: 73 or SEQ ID NO: 74.

Exemplary fusion proteins include fusion proteins having at least 95%, 96%, 97%, 98%, or 99% homology or are identical with SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 75, and SEQ ID NO: 76.

The therapeutic duration of the fusion protein's effect on the concentration of plasma arginine is dependent on the amount of the fusion protein administered and can be about 1, about 2, about 3, about 4, about 5, about 6, about 7, about 8, about 9, about 10, about 11, about 12, about 13, about 14, or about 15 days or more. In certain embodiments, the therapeutic duration of the fusion protein is between about 5 days to about 20 days, about 5 days to about 19 days, about 5 days to about 18 days, about 5 days to about 17 days, about 5 days to about 16 days, about 5 days to about 15 days, about 6 days to about 15 days, about 7 days to about 15 days, about 7 days to about 14 days, about 7 days to about 13 days, about 7 days to about 12 days, about 7 days to about 11 days, or about 8 days to about 11 days.

In certain embodiments, the half-life of the fusion protein is about 1 day to about 10 days, about 1 day to about 9 days, about 1 day to about 8 days, about 1 day to about 7 days, about 1 day to about 6 days, about 1 day to about 5 days, about 1 day to about 4 days, about 1 day to about 3 days, or about 1 day to about 2 days. In the other embodiments, the half-life of the fusion protein is about 6 hours to about 30 hours.

The arginase activity of the fusion proteins described herein can be substantially the same, lower, or higher than the activity of the arginase polypeptide from which it is derived. One unit of arginase activity is defined as the amount of fusion protein [e.g., BHA (SEQ ID NO: 75), BAH (SEQ ID NO: 76), N-ABD-rhArg (SEQ ID NO: 49), or N-ABD094-rhArg (SEQ ID NO: 50)] or arginase [e.g., BCA (SEQ ID NO: 70)] that catalyzes the production of 1 μmol of urea per min under standard assay conditions. The specific activity of the enzyme is expressed as activity units per mg of protein. Under standard diacetylmonoxime (DAMO) assay conditions (37 ° C., pH 7.4), the fusion proteins can have a specific activity that is about 5%, about 10%, about 15%, about 20%, about 25% about 30%, about 35% or about 40% lower or higher than the corresponding arginase polypeptide which it incorporates. In certain embodiments, fusion proteins can have a specific activity that is about 5% to about 40%, about 10% to about 40%, about 10% to about 35%, about 10% to about 30%, about 20% to about 30%, about 20% to about 35%, about 15% to about 30%, about 15% to about 25%, or about 10% to about 20% lower or higher than the corresponding arginase polypeptide which it incorporates.

In certain embodiments, the arginase activity of the fusion proteins is substantially unaffected by the presence of HSA. This is advantageous, because binding of the ABD fusion proteins to HSA can have a deleterious effect on the activity of the fusion protein.

Also provided are polynucleotide sequences encoding the fusion proteins described herein as isolated polynucleotides or as portions of expression vectors or as portions of linear DNA sequences, including linear DNA sequences used for in vitro transcription/translation, vectors compatible with prokaryotic or eukaryotic expression, secretion and/or display of the compositions. Certain exemplary polynucleotides are disclosed herein, however, other polynucleotides which, given the degeneracy of the genetic code or codon preferences in a given expression system, encode the fusion proteins described herein are also within the scope of this disclosure.

The polynucleotides described herein may be produced by chemical synthesis, such as solid phase polynucleotide synthesis on an automated polynucleotide synthesizer and assembled into complete single or double stranded molecules. Alternatively, the polynucleotides of the invention may be produced by other techniques, such as a PCR followed by routine cloning. Techniques for producing or obtaining polynucleotides of a given known sequence are well known in the art.

The polynucleotides of the described herein may comprise at least one non-coding sequence, such as a promoter or enhancer sequence, intron, polyadenylation signal, and the like. The polynucleotide sequences may also comprise additional sequences encoding additional amino acids that encode for example a marker or a tag sequence, such as a poly-histidine (6× His) or an HA tag to facilitate purification or detection of the protein, a signal sequence, a fusion protein partner, such as cDNA encoding a bioactive agent, and the like.

In another embodiment, provided herein is a vector comprising at least one of the polynucleotides described herein. Such vectors may be plasmid vectors, viral vectors, vectors for baculovirus expression, transposon based vectors or any other vector suitable for introduction of the polynucleotides of the invention into a given organism or genetic background by any means. Such vectors may be expression vectors comprising nucleic acid sequence elements that can control, regulate, cause or permit expression of a polypeptide encoded by such a vector. Such elements may comprise transcriptional enhancer binding sites, RNA polymerase initiation sites, ribosome binding sites, and other sites that facilitate the expression of encoded polypeptides in a given expression system. Such expression systems may be cell-based, or cell-free systems well known in the art.

In many bacterial expression systems, the start codon typically codes for methionine, which consequently produces proteins initiated with a N-terminal methionine in these expression systems. However, it is well known that certain bacterial enzymes, such as methionine aminopeptidase (MetAP) and the like, can catalyze the hydrolytic cleavage of the N-terminal methionine from newly synthesized polypeptides. This is commonly observed in instances in which the next amino acid is, e.g., Gly, Ala, Ser, or Thr [In vivo processing of N-terminal methionine in E. coli, FEBS Lett. 1990 Jun. 18; 266(1-2):1-3]. Accordingly, in certain embodiments of the fusion proteins described herein include variants in which the N-terminal methionine of the protein is not present.

The fusion proteins described herein can be isolated using separation procedures well known in the art for capture, immobilization, partitioning, or sedimentation, and purified to the extent necessary for commercial applicability.

For therapeutic use, the fusion proteins described herein may be prepared as pharmaceutical compositions containing a therapeutically effective amount of a fusion protein described herein as an active ingredient in a pharmaceutically acceptable carrier. The term “carrier” refers to a diluent, adjuvant, excipient, or vehicle with which the active compound is administered. Such vehicles can be liquids, such as water and oils, including those of petroleum, animal, vegetable or synthetic origin, such as peanut oil, soybean oil, mineral oil, sesame oil and the like. For example, 0.9% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the fusion protein in such pharmaceutical formulation can vary widely, e.g., from less than about 0.5%, usually at or at least about 1% to as much as 15 or 20% by weight and will be selected primarily based on required dose, fluid volumes, viscosities, etc., according to the particular mode of administration selected. Suitable vehicles and formulations are described, for example, in Remington: The Science and Practice of Pharmacy, 21st Edition, Troy, D. B. ed., Lipincott Williams and Wilkins, Philadelphia, Pa. 2006, Part 5, Pharmaceutical Manufacturing pp 691-1092, See especially pp. 958-989.

The mode of administration for therapeutic use of the fusion protein described herein may be any suitable route that delivers the agent to the host, such as parenteral administration, e.g., intradermal, intramuscular, intraperitoneal, intravenous or subcutaneous, pulmonary; transmucosal (oral, intranasal, intravaginal, rectal); using a formulation in a tablet, capsule, solution, suspension, powder, gel, particle; and contained in a syringe, an implanted device, osmotic pump, cartridge, micropump; or other means appreciated by the skilled artisan, as well known in the art. Site specific administration may be achieved by for example intrarticular, intrabronchial, intraabdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery.

The concentration of plasma arginine in the subject needed to observe a therapeutic effect can vary based on numerous factors, including the condition of the subject and the type and severity of the disease and/or medical condition and/or diet composition. The selection of the target plasma arginine levels is well within the skill of a person of ordinary skill in the art. In certain embodiments, the concentration of plasma arginine is below about 100 μM, about 90 μM, about 80 μM, about 70 μM, about 60 μM, about 50 μM, about 40 μM, about 30 μM, about 20 μM, about 10 μM, or about 5 μM. In certain embodiments, the concentration of plasma arginine is about 0.1 μM to about 100 μM, about 0.1 μM to about 90 μM, about 0.1 μM to about 80 μM, about 0.1 μM to about 70 μM, about 0.1 μM to about 60 μM, about 0.1 μM to about 50 μM, about 0.1 μM to about 40 μM, about 0.1 μM to about 30 μM, about 0.1 μM to about 20 μM, or about 0.1 μM to about 10 μM. In certain embodiments, the level of arginine is below the detection limit of the Biochrom 30 Amino Acid Analyzer (e.g., below about 3 μM) and/or below the detection limit of the Agilent 6460 Liquid Chromatography/Electrospray Ionization Triple Quadrupole Mass Spectrometer (e.g., lower than about 0.3 μM).

The determination of the duration of treatment, e.g., the duration of time the plasma arginine concentrations are maintained in a depleted state in the subject, is well within the skill of a person of ordinary skill in the art. In certain embodiments, the duration of treatment is about 1, about 2, about 3, about 4, about 8, about 12, about 16, about 20, about 24, about 28, about 32, about 36, about 40, about 44, about 48, about 52, about 56 weeks, or longer.

In certain embodiments, the method of inducing intermittent fasting, modulating autophagy, or inducing intermittent fasting and modulating autophagy in a subject in need thereof comprises co-administering a therapeutically effective amount of an arginine depleting agent and therapeutically effective amount of an autophagy inducing agent to the subject.

Any autophagy inducing agent known in the art can be used in the methods described herein. Exemplary autophagy inducing agents include, but are not limited to, carbamazepine, clonidin, lithium, metformin, rapamycin (and rapalogs), rilmenidine, sodium valproate, verapamil, trifluoperazine, statins, tyrosine kinase inhibitors, BH3 mimetics, caffeine, omega-3 polyunsaturated fatty acids, resveratrol, spermidine, vitamin D, trehalose, polyphenol(−)-epigallocatechin-3-gallate and combinations thereof.

In certain embodiments, the method of inducing intermittent fasting, modulating autophagy, or inducing intermittent fasting and modulating autophagy in a subject in need thereof comprises co-administering a therapeutically effective amount of an arginine depleting agent and therapeutically effective amount of a glucose lowering agent to the subject.

In certain embodiments, the glucose lowering agent is an alpha-glucosidase inhibitor, a biguanide, bile acid sequestrant, a dopamine-2 agonist, a dipeptidyl peptidase 4 (DPP-4) inhibitor, a meglitinide, a sodium-glucose transport protein 2 (SGLT2) inhibitor, a sulfonylurea, a thiazolidinedione, or a combination thereof.

In certain embodiments, the biguanide is metformin; the alpha-glucosidase inhibitor is acarbose or miglitol; the bile acid sequestrant is colesevelam; the dopamine-2 agonist is bromocriptine; the DPP-4 inhibitor is alogliptin, linagliptin, saxagliptin, or sitagliptin; the meglitinide is nateglinide or repaglinide; the SGLT2 inhibitor is canagliflozin, dapagliflozin, or empagliflozin; the sulfonylureas ischlorpropamide, glimepiride, glipizide, or glyburide; and the thiazolidinedione is rosiglitazone or pioglitazone.

In certain embodiments, the method of inducing intermittent fasting, modulating autophagy, or inducing intermittent fasting and modulating autophagy in a subject in need thereof comprises co-administering a therapeutically effective amount of an arginine depleting agent and a therapeutically effective amount of a retinoid derivative to the subject.

In certain embodiments, the retinoid derivative is acitretin, alitretinoin bexarotene, isotretinoin, retinol, retinoic acid, or a pharmaceutically acceptable salt thereof. In certain embodiments, the retinoid derivative is retinoic acid.

In certain embodiments, the method of inducing intermittent fasting, modulating autophagy, or inducing intermittent fasting and modulating autophagy in a subject in need thereof comprises co-administering a therapeutically effective amount of an arginine depleting agent and a therapeutically effective amount of green tea catechin (−)-epigallocatechin-3-gallate (EGCG) derivative or a pharmaceutically acceptable salt or product thereof to the subject. In certain embodiments, the EGCG derivative is EGCG or pharmaceutically acceptable salt thereof or EGCG peracetate. In certain embodiments, the green tea catechin is (−)-epicatechin (EC), (−)-epicatechin-3-gallate (ECG), (−)-epigallocatechin (EGC), and their derivatives.

In certain embodiments, the method of inducing intermittent fasting, modulating autophagy, or inducing intermittent fasting and modulating autophagy in a subject in need thereof comprises co-administering a therapeutically effective amount of an arginine depleting agent and a therapeutically effective amount of a rapamycin or rapamycin derivative to the subject.

The arginine depleting agents can be administered according to therapeutic protocols well known in the art. It will be apparent to those skilled in the art that the administration of the arginine depleting agents and the autophagy inducing agent can be varied depending on the disease or health condition being treated and the known effects of the autophagy inducing agent on that disease or health condition. Also, in accordance with the knowledge of the skilled clinician, the therapeutic protocols (e.g., dosage amounts and times of administration) can be varied in view of the observed effects of the administered therapeutic agents (i.e., autophagy inducing agent) on the subject, and in view of the observed responses of the disease to the administered therapeutic agents.

Also, in general, arginine depleting agents and the autophagy inducing agent do not have to be administered in the same pharmaceutical composition, and may, because of different physical and chemical characteristics, have to be administered by different routes. For example, arginine depleting agents may be administered intravenously to generate and maintain good blood levels, while the autophagy inducing agent may be administered orally. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice of autophagy inducing agent will depend upon the diagnosis of the attending physicians and their judgment of the condition of the subject and the appropriate treatment protocol.

An arginine depleting agent and autophagy inducing agent may be administered concurrently (e.g., simultaneously, essentially simultaneously or within the same treatment protocol) or sequentially, depending upon the nature of the disease or health condition under treatment, the condition of the subject, and the actual choice of autophagy inducing agent to be administered in conjunction (i.e., within a single treatment protocol) with an arginine depleting agent.

If an arginine depleting agent and the autophagy inducing agent are not administered simultaneously or essentially simultaneously, then the optimum order of administration of the arginine depleting agent and the autophagy inducing agent, may be different for different diseases or health conditions Thus, in certain situations the arginine depleting agent may be administered first followed by the administration of the autophagy inducing agent; and in other situations the autophagy inducing agent may be administered first followed by the administration of an arginine depleting agent. This alternate administration may be repeated during a single treatment protocol. The determination of the order of administration, and the number of repetitions of administration of each therapeutic agent during a treatment protocol, is well within the knowledge of the skilled physician after evaluation of the disease or health condition being treated and the condition of the subject. For example, the autophagy inducing agent may be administered first and then the treatment continued with the administration arginine depleting agent followed, where determined advantageous, by the administration of the autophagy inducing agent, and so on until the treatment protocol is complete.

Thus, in accordance with experience and knowledge, the practicing physician can modify each protocol for the administration of a component (arginine depleting agent and autophagy inducing agent) of the treatment according to the individual patient's needs, as the treatment proceeds.

### EXAMPLES

### Example 1

### The N-ABD094-rhArg (SEQ ID NO: 50) Induces Repetitive Cycles of Intermittent Fasting

The recombinant human arginase [N-ABD094-rhArg (SEQ ID NO: 50)] is a novel, engineered long-acting arginine-depleting enzyme. Treatment with N-ABD094-rhArg (SEQ ID NO: 50) once a week can induce repetitive 7-day intermittent fasting cycles composed of fasting and refeeding period.

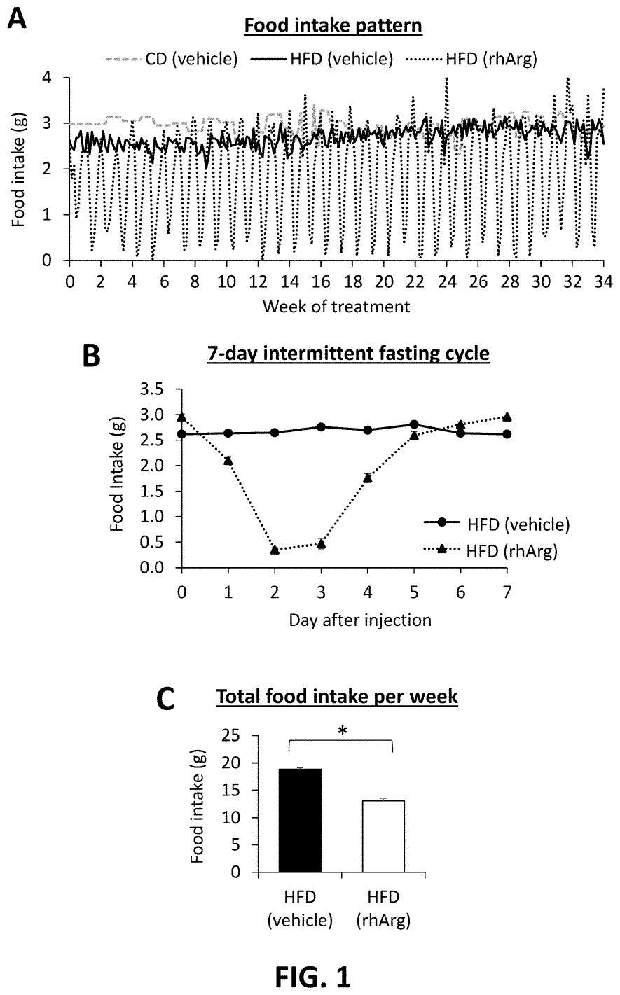

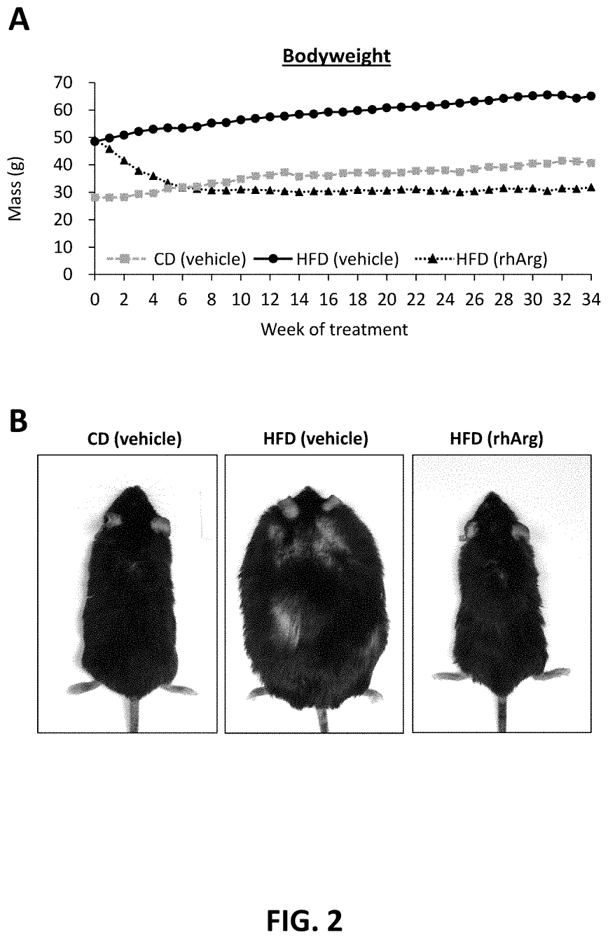

One of the most noticeable effects of intermittent fasting is reduction of bodyweight. To demonstrate this effect, a diet-induced obesity mouse model [Wang and Liao, 2012, Methods Mol. Biol. 821:421-433], which shares many characteristics with human obesity and is widely used for testing prospective anti-obesity agents [Vickers et al., 2011, Br. J. Pharmacol. 164(4):1248-1262], was employed. Specifically, C57BL/6J, which is the most commonly used mouse strain for polygenic obesity model, was used in the study. It is well-reported that C57BL/6J male mice feeding on a high-fat diet will have prominent weight gain, become obese and develop hyperinsulinemia, hyperglycemia, impaired glucose tolerance and insulin resistance [Gallou-Kabani et al., 2007, Obesity 15(8):1996-2005]. Obesity was induced in C57BL/6J male mice via feeding ad libitum a high-fat diet containing 60 kcal % fat (HFD; Research Diets, D12492) for 12-14 weeks starting from 5-week old. These mice are referred as diet-induced obese (DIO) mice. DIO mice were stratified according to bodyweight into 2 groups. One group received intraperitoneal (i.p.) injection of about 600 U of N-ABD094-rhArg (SEQ ID NO: 50) (rhArg) dissolved in saline [HFD (rhArg) group], while another group received vehicle (saline) [HFD (vehicle) group] once a week for 34 weeks. Both groups of mice continued to feed ad libitum on the HFD throughout the treatment period. One group of age-matched C57BL/6J male mice feeding ad libitum on an ordinary rodent chow diet (CD; LabDiet, 50IF/6F) and injected with vehicle served as the lean control [CD (vehicle) group]. Food intake and bodyweight of mice were measured daily. As shown in FIG. 1A, while the daily food intake of DIO and lean control mice treated with vehicle was relatively stable, DIO mice treated with rhArg exhibited repetitive cycles of intermittent fasting throughout the treatment period. FIG. 1B illustrates the pattern of a 7-day intermittent fasting cycle, showing the average food intake on each day of the cycle. DIO mice were injected with rhArg on Day 0, which was Day 7 of the previous cycle. Similarly, Day 7 of the cycle shown in FIG. 1B was Day 0 of the next cycle. The intermittent fasting cycle is composed of fasting and refeeding period. For a typical cycle exhibited by the cohort of mice shown in FIG. 1B, there was mild reduction of food intake in the first day after rhArg administration. Marked reduction of food intake (fasting) occurred in the following 2 days, following by a gradual increase of food intake (refeeding). On average, mice treated with rhArg had food intake reduced by about 30% (FIG. 1C) in a week. Concomitant with induction of repetitive intermittent fasting cycles, administration of rhArg could effectively lead to substantial weight loss, such that within 6-7 weeks of treatment, the bodyweight of DIO mice had reduced by 40% (from 50 g to 30 g), and became similar to the bodyweight of the lean control mice (FIG. 2A). For the rest of the treatment period, the bodyweight of mice administered with rhArg was well-maintained at around 30 g despite continued feeding on a HFD (FIG. 2B). In contrast, not only DIO mice fed the HFD and administered with vehicle progressively increased in bodyweight from 50 g to 66 g during 34-week treatment period, the lean control mice also exhibited bodyweight gain from 28 g to 42 g during the 34-week treatment period. Bodyweight gain is one of the biomarkers related to aging. Together, these results demonstrate that rhArg has potent anti-obesity effect.

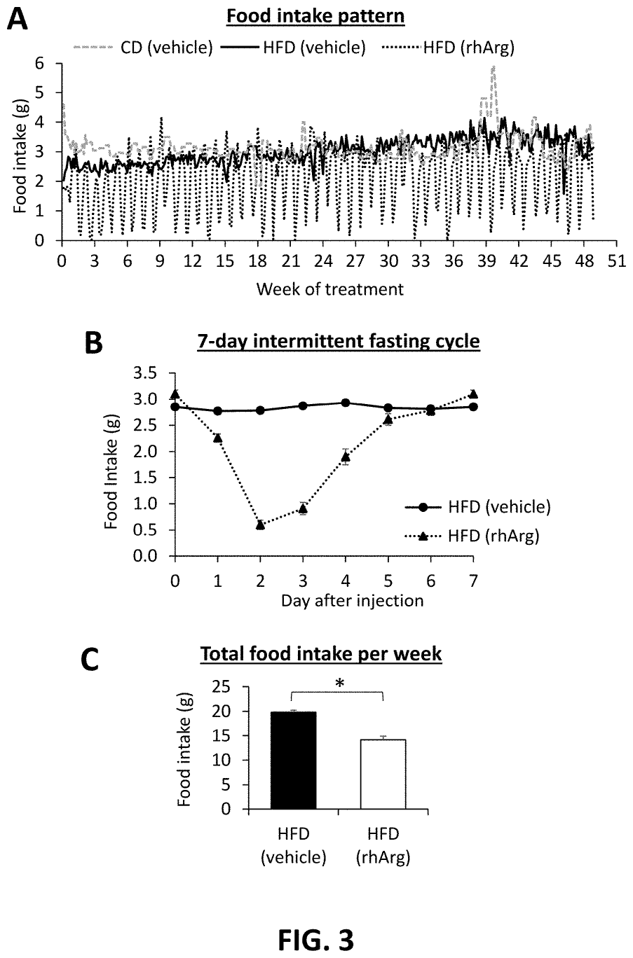

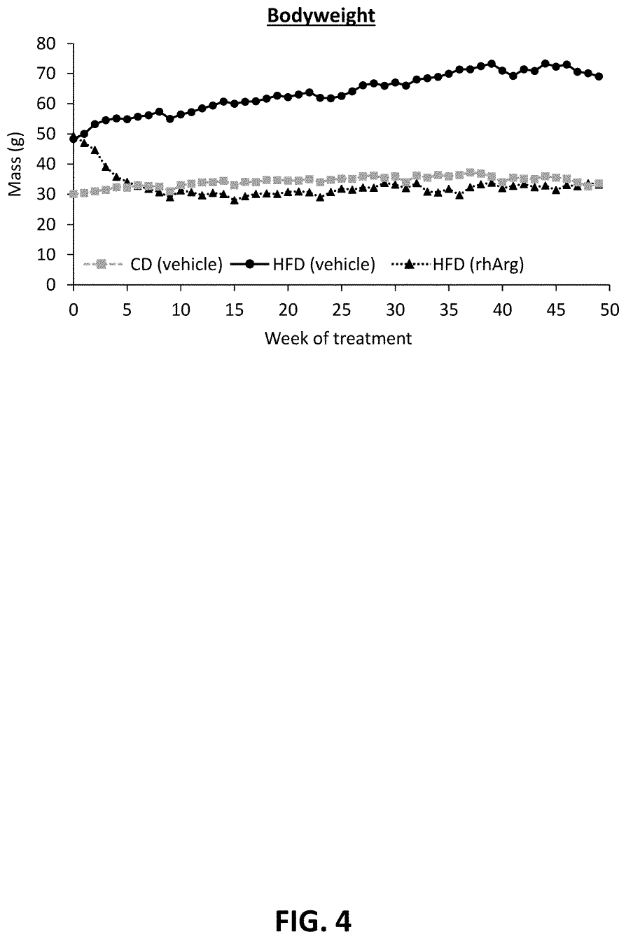

To determine whether the effects of rhArg on induction of intermittent fasting and regulating bodyweight are reproducible, rhArg was administered to an independent cohort of DIO and lean control C57BL/6J male mice and the treatment period was extended to 49 weeks. As shown in FIG. 3A, DIO mice administered with rhArg once a week underwent repetitive cycles of intermittent fasting throughout the treatment period. The pattern of the 7-day intermittent fasting cycle (FIG. 3B) and the resulting reduction of weekly food intake by about 29% (FIG. 3C) highly resembled that occurred in the aforementioned cohort of mice that were treated with rhArg for 34 weeks (FIG. 1). Similarly, concomitant with induction of repetitive cycles of intermittent fasting, the bodyweight of this cohort of mice reduced from 50 g to 30 g within 6-7 weeks of rhArg administration, and then remained relatively constant throughout the rest of the treatment period (FIG. 4). These results demonstrate that the effects of rhArg are reproducible.

### Example 2

### The N-ABD094-rhArg (SEQ ID NO: 50) does not Induce Drug Resistance

As shown in FIG. 1A, DIO mice underwent the intermittent fasting cycle throughout the 34-week treatment period, which suggested that mice remained susceptible to the effect of rhArg without the development of drug resistance.

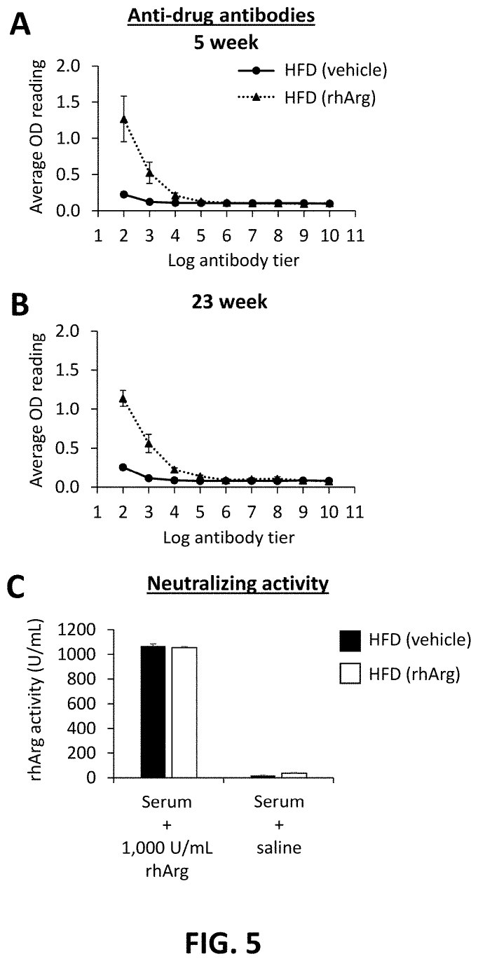

Generation of neutralizing antibodies is a common cause of development of resistance to protein drug. Using the enzyme-linked immunosorbent assay (ELISA) to detect antibodies against N-ABD094-rhArg (SEQ ID NO: 50) in the mouse serum taken at 5 and 23 weeks after weekly injection, it was found that anti-rhArg antibodies were detectable at 5 weeks (FIG. 5A) and the antibody tier remained the same at 23 weeks (FIG. 5B). However, despite the presence of anti-rhArg antibodies in the serum, when a fixed concentration of rhArg (1,000 U/mL) was incubated with the serum, followed by measurement of the enzymatic activity of rhArg, there was no reduction in the enzymatic activity of rhArg (FIG. 5C), which implied that the anti-rhArg antibodies in the serum did not have neutralizing effects, and thus suggesting that the anti-drug antibodies generated did not interfere with the active site of the drug molecule.

Together, these results demonstrate that N-ABD094-rhArg (SEQ ID NO: 50) does that induce drug resistance and is suitable for long-term usage.

### Example 3

### The N-ABD094-rhArg (SEQ ID NO: 50) Induces Intermittent Fasting and Reverses Adiposity in Diet-Induced Obese C57BL/6J Male Mice

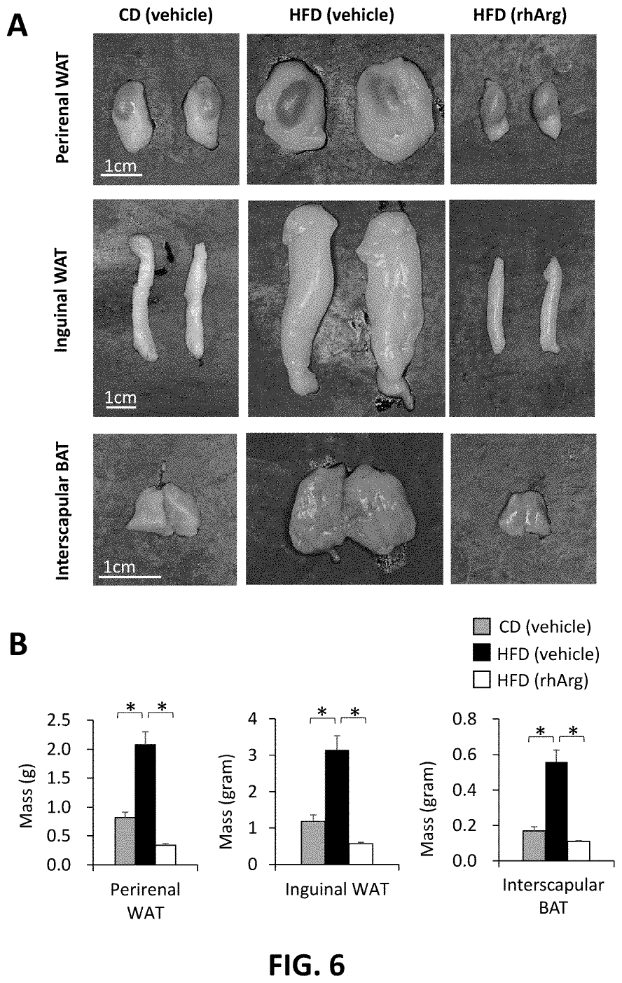

Intermittent fasting has been shown to reduce obesity, hypertension, and bring metabolic benefits As shown in FIG. 6, concomitant with induction of repetitive cycles of intermittent fasting (FIG. 1) and substantial weight loss (FIG. 2), the mass of the perirenal fat pad (representative depot of visceral white adipose tissue WAT) (FIG. 6A), inguinal fat pad (representative depot of subcutaneous WAT) (FIG. 6B) and interscapular fat pad (representative depot of brown adipose tissue BAT) (FIG. 6C) in DIO mice treated with rhArg was less than 20% of that in DIO mice treated with vehicle.

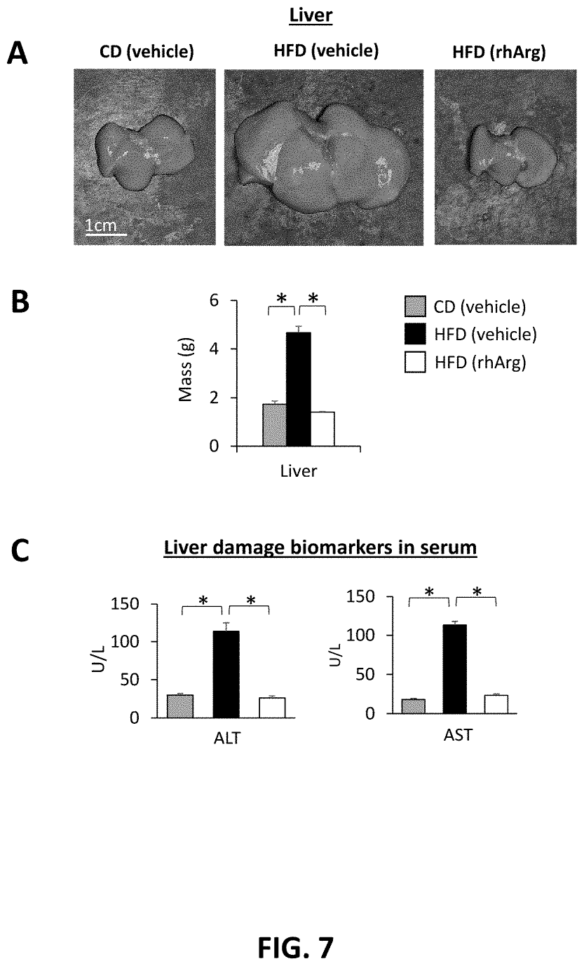

Other than WAT and BAT, obesity is associated with excessive accumulation of lipids in the liver (hepatic steatosis). Indeed, the liver of vehicle-treated DIO mice was markedly enlarged (FIG. 7A), with a mass about 3 times (4.68 g) that of the lean control (1.73 g) (FIG. 7B). However, in DIO mice treated with rhArg, the liver mass was dramatically reduced to 1.39 g (FIG. 7B). Serum concentrations of alanine transaminase (ALT) and aspartate transaminase (AST), two commonly used biomarkers of liver damage, were restored to levels comparable to the lean control mice.

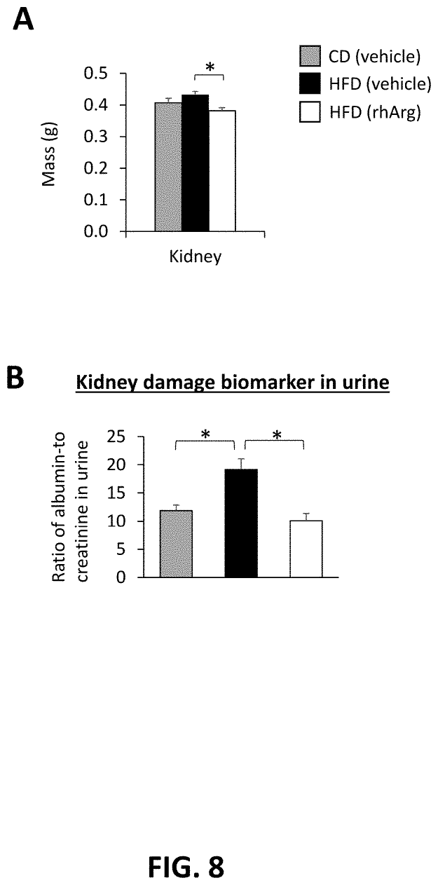

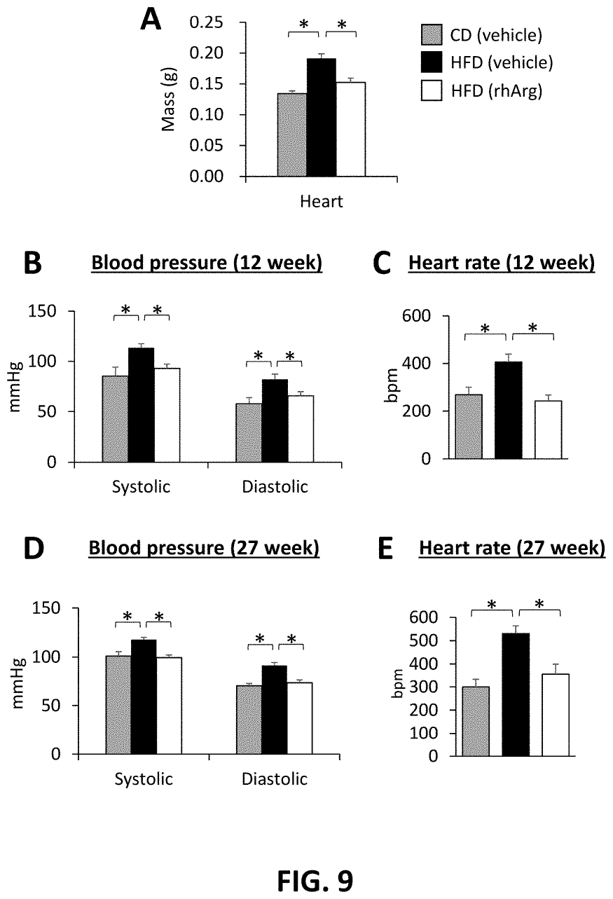

Other than liver, obesity is also commonly associated with lipid accumulation in the kidney (renal steatosis) and heart (cardiac steatosis), causing lipotoxicity and dysfunction of these organs. Indeed, there was an increase in kidney and heart mass in DIO mice treated with vehicle (FIG. 8A and FIG. 9A). Ratio of albumin-to-creatinine in urine, commonly used as biomarker of kidney damage, was significantly increased (FIG. 8B). Systolic and diastolic blood pressure (FIG. 9B), and heart rate (FIG. 9C) were significantly higher in vehicle-treated DIO mice in comparison to the lean control mice. Notably, in DIO mice receiving once weekly treatment of rhArg, their kidney (FIG. 8A) and heart mass (FIG. 9A) was significantly less than that of the vehicle-treated DIO mice. Ratio of albumin-to-creatinine in urine (FIG. 8B), blood pressure and heart rate measured at 12 weeks (FIGS. 9B and C) and 27 weeks (FIGS. 9D and E), were all at levels similar to the age-matched lean control mice.

Together, these findings support that rhArg can effectively reverse adiposity and protect major organs including the liver, kidney and heart from obesity-related dysfunctions and diseases, and prevent against hypertension, kidney and liver damages despite continual intake of a high-fat diet.

### Example 4

### Treatment with N-ABD094-rhArg (SEQ ID NO: 50) Reverses HFD-Induced Insulin Resistance and Impaired Glucose Tolerance

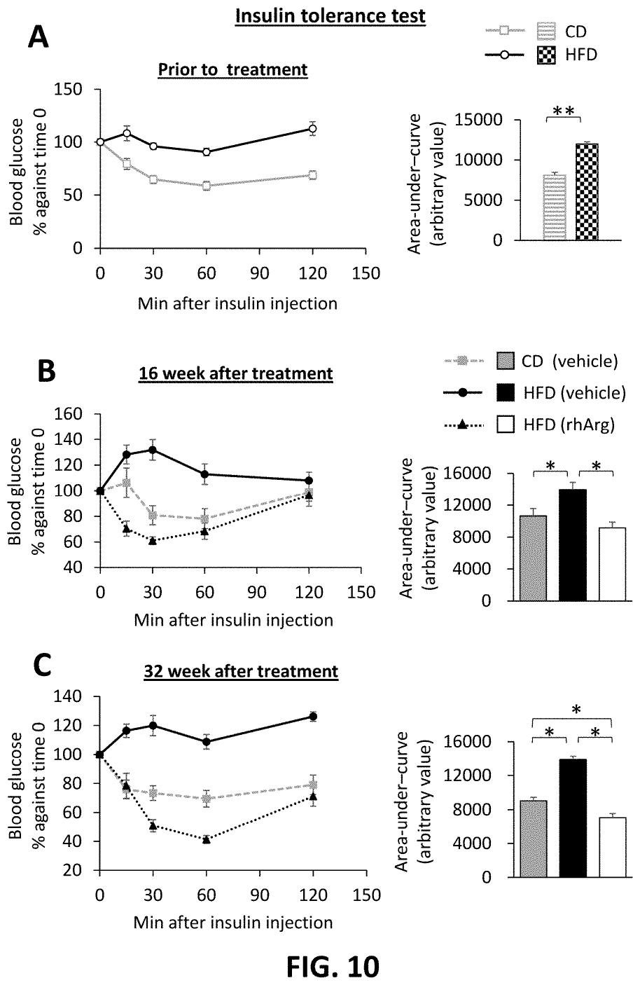

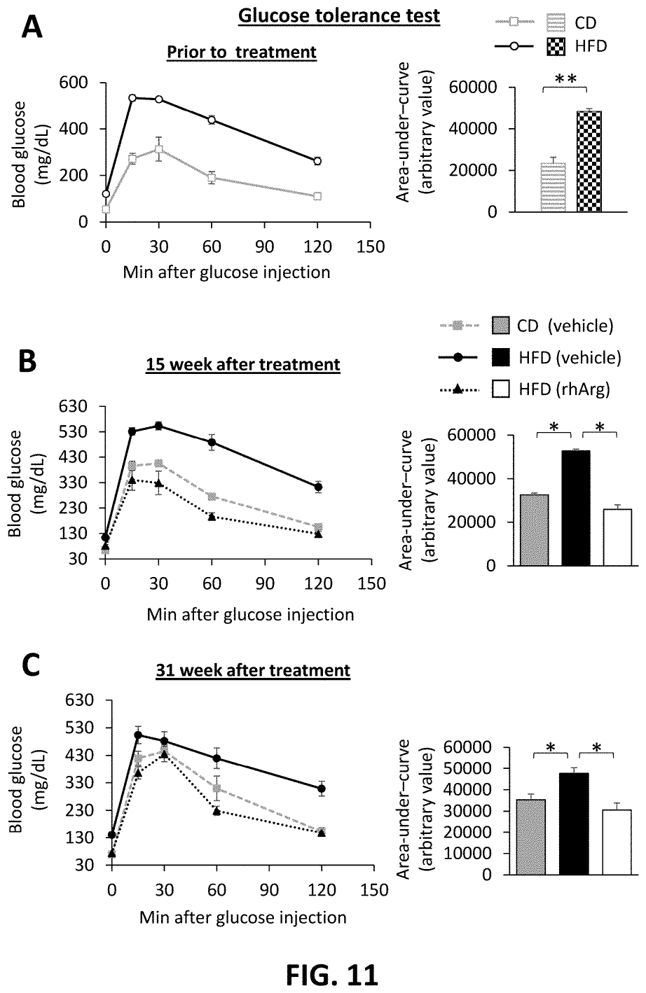

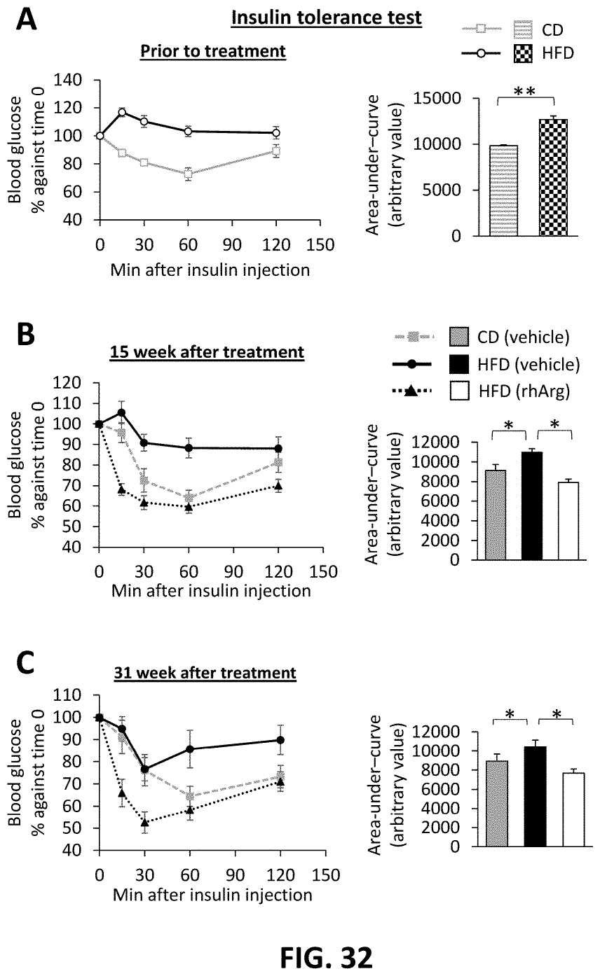

Intermittent fasting has been shown to have benefits on type 2 diabetes. C57BL/6J male mice feeding on a HFD for 12 weeks would develop peripheral insulin resistance (FIG. 10A) and impaired glucose tolerance (FIG. 11A), which are characteristics of prediabetes. However, DIO mice showed complete reversion of insulin resistance and exhibited marked increase in insulin sensitivity when subjected to insulin tolerance test at 16 (FIG. 10B) and 32 weeks (FIG. 10C) after receiving weekly treatment of N-ABD094-rhArg (SEQ ID NO: 50). In fact, rhArg-treated mice fed a HFD consistently (FIGS. 10B and 10C) had better insulin sensitivity than age-matched vehicle-treated mice fed a chow diet.

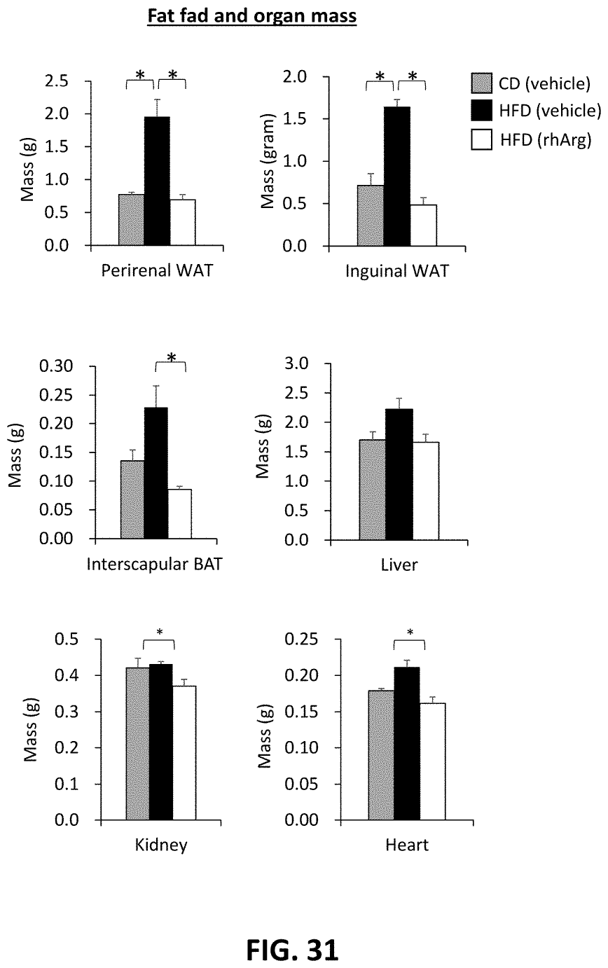

Besides insulin sensitivity, mice treated with N-ABD094-rhArg (SEQ ID NO: 50) exhibited significant improvement in glucose tolerance to a level comparable with the lean control mice when tested at 15 (FIG. 11B) and 31 weeks (FIG. 11C) after rhArg treatment.

Collectively, our findings demonstrate the efficacy of N-ABD094-rhArg (SEQ ID NO: 50) in enhancing insulin sensitivity and glucose tolerance, and it has great potential to be applied as an insulin-sensitizing agent for therapeutic treatment of prediabetes and type 2 diabetes characterized by insulin resistance, glucose intolerance and other disorders associated with insulin resistance.

### Example 5

### Artificial Intermittent Fasting by Controlled Feeding, but not Daily Reduced Food Intake can Induce Similar Anti-Obesity Effects as N-ABD094-rhArg (SEQ ID NO: 50)

Mice administered with N-ABD094-rhArg (SEQ ID NO: 50) have reduced food intake of around 30% per week. To decipher whether the anti-obesity effects of rhArg are caused by intermittent fasting and/or reduction in food intake, C57BL/6J male mice were subjected to the following feeding protocol:

C57BL/6J male mice were fed a high-fat diet from 5-week old for 12 weeks. They were then stratified into 3 groups according to their bodyweight and individually caged: (i) one group of mice received i.p. injection of 600 U N-ABD094-rhArg (SEQ ID NO: 50) once a week [HFD (rhArg) group] and was fed ad libitum the HFD for 5 weeks; (ii) one group of mice received i.p. injection of saline once a week, and was fed a predetermined amount of HFD on each day to artificially create a 7-day intermittent fasting cycle that mimics the pattern of food intake of mice administered once a week with rhArg [HFD (artificial IF) group)]; (iii) one group of mice was injected with saline once a week and was fed ad libitum the HFD [HFD (vehicle) group].

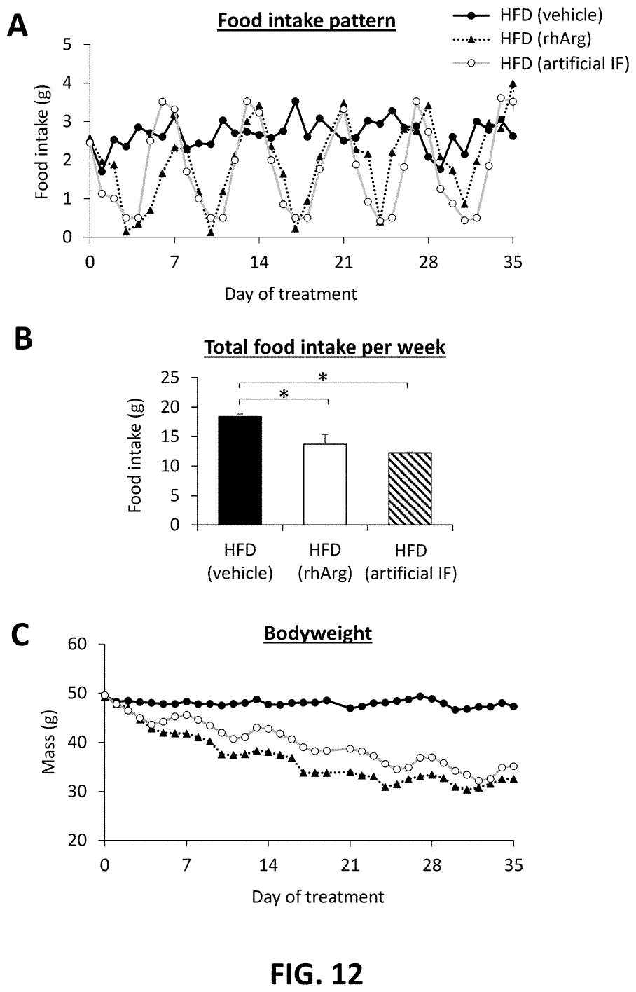

The food intake pattern of the 3 groups of mice and total food intake per week were shown in FIGS. 12A and 12B respectively. Results showed that mice that underwent 7-day cycles of intermittent fasting, achieved via rhArg treatment or artificial predetermined feeding protocol, exhibited substantial weight loss over the 5-week period. The pattern of change in bodyweight was similar, but the rate of weight loss exhibited by the HFD (rhArg) group was faster than HFD (artificial IF) group during the first 3 weeks of treatment. At the end of 5 weeks of treatment, both groups of mice showed marked reduction in the fat pad mass of perirenal and inguinal WAT, and interscapular BAT of mice, with the extent of reduction of WAT greater for HFD (rhArg) group. The liver mass was prominently reduced to the same extent for both groups.

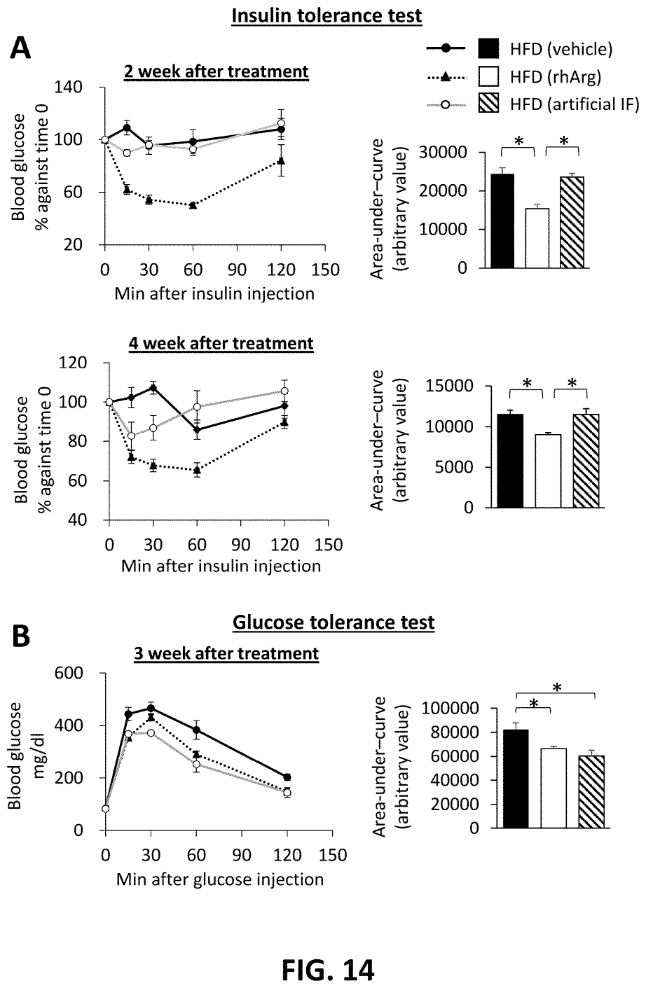

However, despite showing a prominent effect on reducing bodyweight and fat mass, mice undergoing artificial intermittent fasting cycles did not show significant improvement in insulin sensitivity in the insulin tolerance test conducted at 2 weeks and 4 weeks after treatment (FIG. 14A). In contrast, mice receiving weekly injection of rhArg exhibited reversal of insulin resistance and marked increase in insulin sensitivity by 2 weeks after treatment, and such improvement was well maintained when ITT test was repeated at 4 weeks after treatment. On the other hand, both groups of mice showed significant improvement in glucose tolerance by 3 weeks of treatment (FIG. 14B). Together, these results suggest that the effect of N-ABD094-rhArg (SEQ ID NO: 50) on improving glucose tolerance is probably brought about by bodyweight reduction and/or intermittent fasting, whereas its strong potency in increasing insulin sensitivity is mediated via other mechanisms.

To further decipher whether the beneficial effects of N-ABD094-rhArg (SEQ ID NO: 50) are brought by reduced food intake and/or intermittent fasting, DIO male mice were stratified into 3 groups according to their bodyweight and individually caged: (i) one group of mice received i.p. injection of 600 U N-ABD094-rhArg (SEQ ID NO: 50) once a week [HFD (rhArg) group] and was fed ad libitum the HFD for 5 weeks; (ii) one group of mice received i.p. injection of saline once a week, and was fed daily with 2.0 g of HFD (about 70% of average daily food intake by DIO mice) [HFD (reduced) group]; (iii) one group of mice was injected with saline once a week and was fed ad libitum the HFD [HFD (vehicle) group].

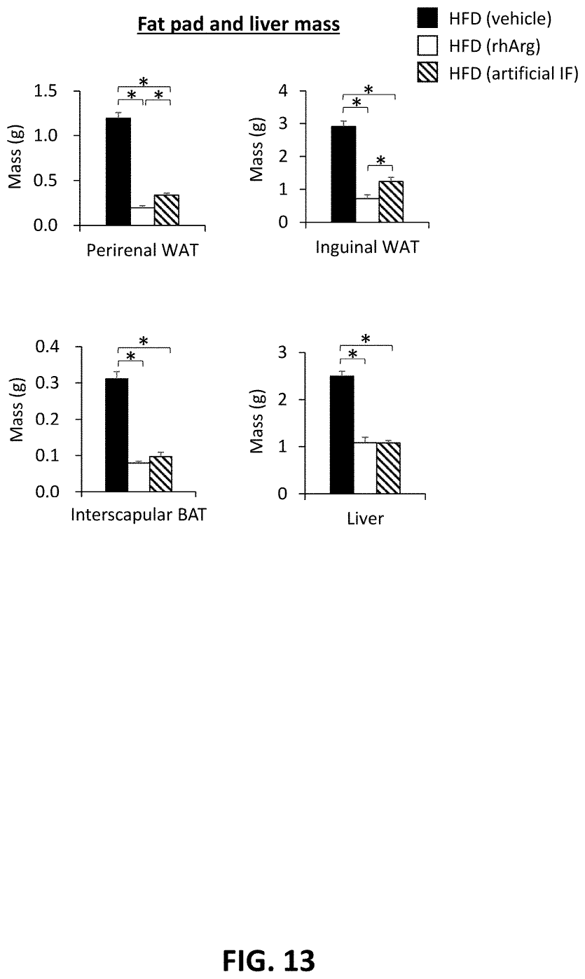

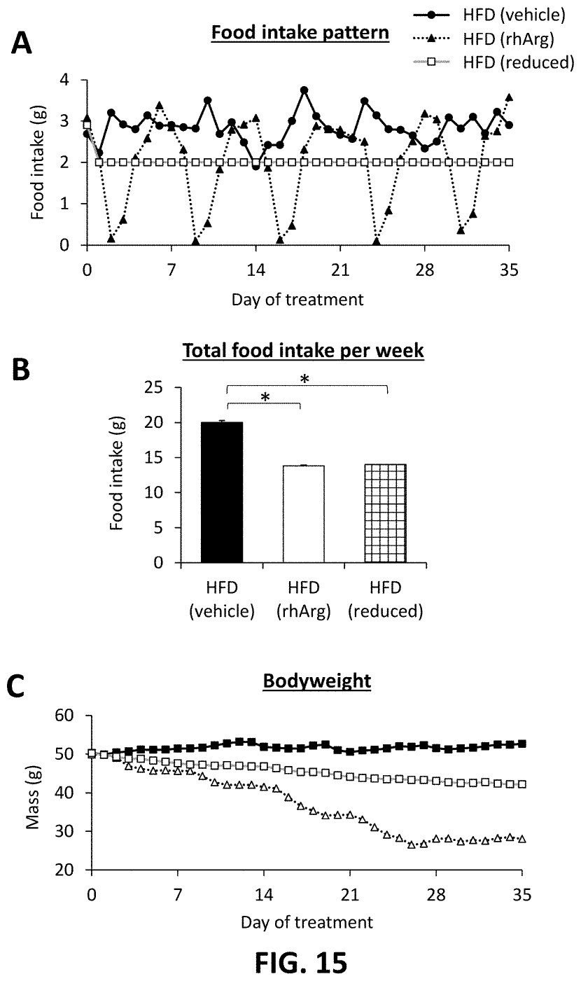

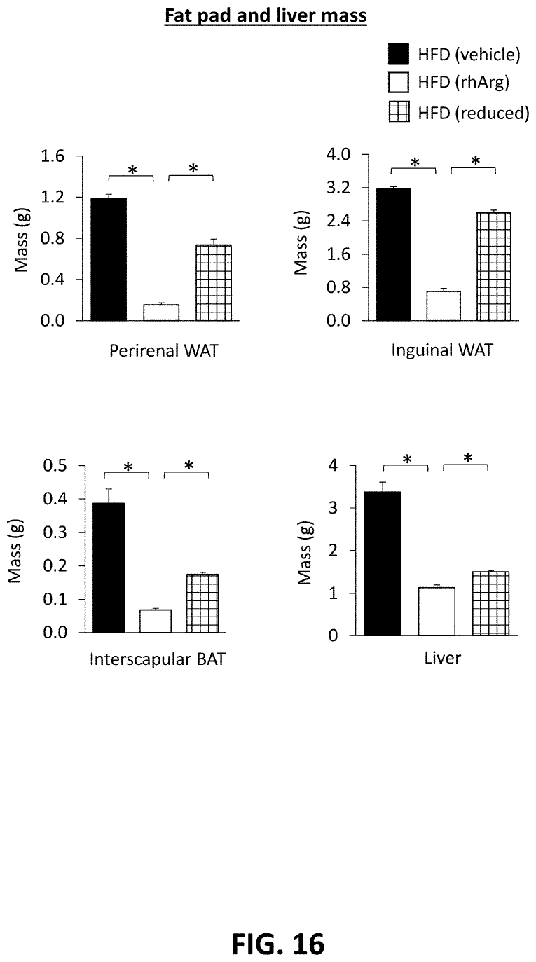

The food intake pattern of the 3 groups of mice and total food intake per week were shown in FIGS. 15A and 15B respectively. Results showed that despite the fact that both HFD (rhArg) group and HFD (reduced) group of mice had 30% reduction of total food intake per week, HFD (rhArg) mice that underwent a 7-day intermittent fasting cycle showed more prominent and rapid weight loss than HFD (reduced) group (FIG. 15C). As a result, by the end of 5 weeks of treatment, the bodyweight of HFD (rhArg) mice had decreased by 44%, whereas HFD (reduced) group only showed 16% reduction in bodyweight. In line with this finding, there were marked differences between these two groups of mice in the extent of reduction in fat pad mass of perirenal and inguinal WAT, and interscapular BAT. Such differences between HFD (rhArg) and HFD (reduced) groups were much greater than the differences between HFD (rhArg) and HFD (artificial IF) groups (FIG. 13), which implies that the anti-obesity effect of intermittent fasting is not merely due to reduction in food intake.

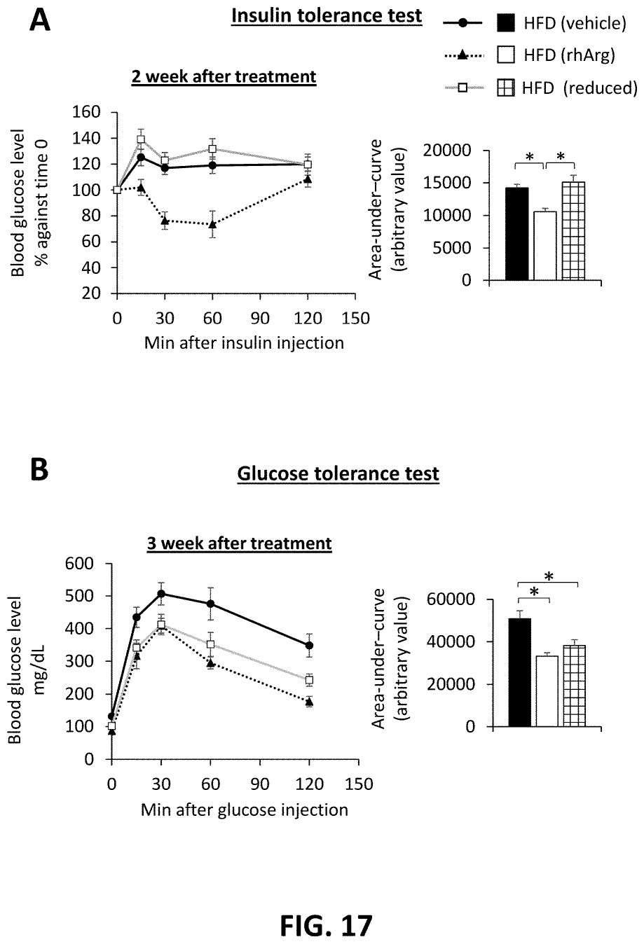

Similar to previous findings, in this experiment, mice receiving weekly injection of rhArg showed marked increase in insulin sensitivity by 2 weeks after treatment (FIG. 17A), and had improved glucose tolerance (FIG. 17B). In contrast, mice with daily reduced food intake only exhibited significant improvement in glucose tolerance, but remained insulin resistant.

Collectively, results of these two independent experiments support that induction of intermittent fasting plays an important role in mediating the anti-obesity effect of N-ABD094-rhArg (SEQ ID NO: 50).

### Example 6

### Different Arginine Depleting Agents can Induce Intermittent Fasting and Reduce Bodyweight of C57BL/6J Male Mice Fed with HFD

To determine whether or not induction of intermittent fasting is restricted to the effect of N-ABD094-rhArg [SEQ ID NO: 50], other arginine depleting agents were examined.

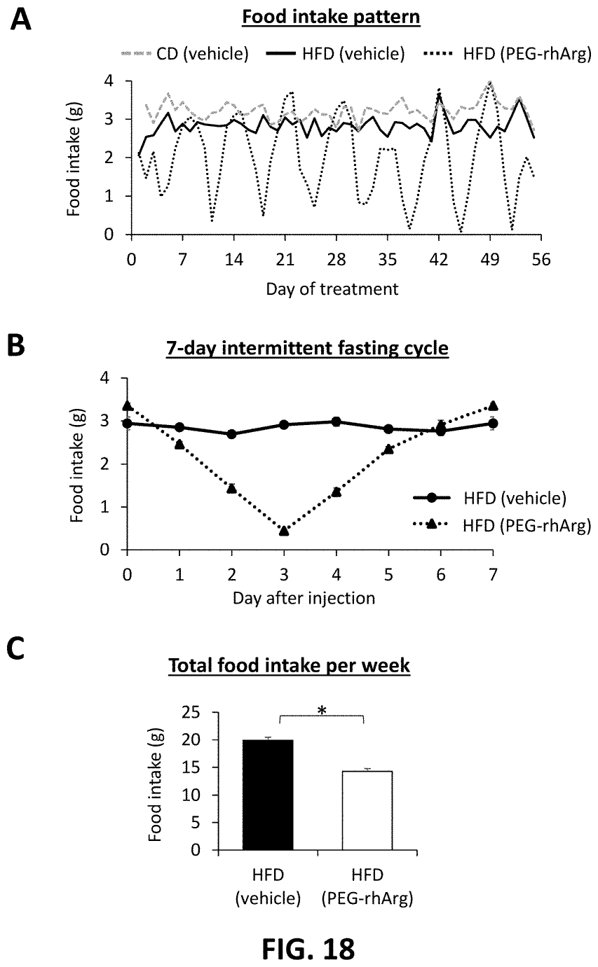

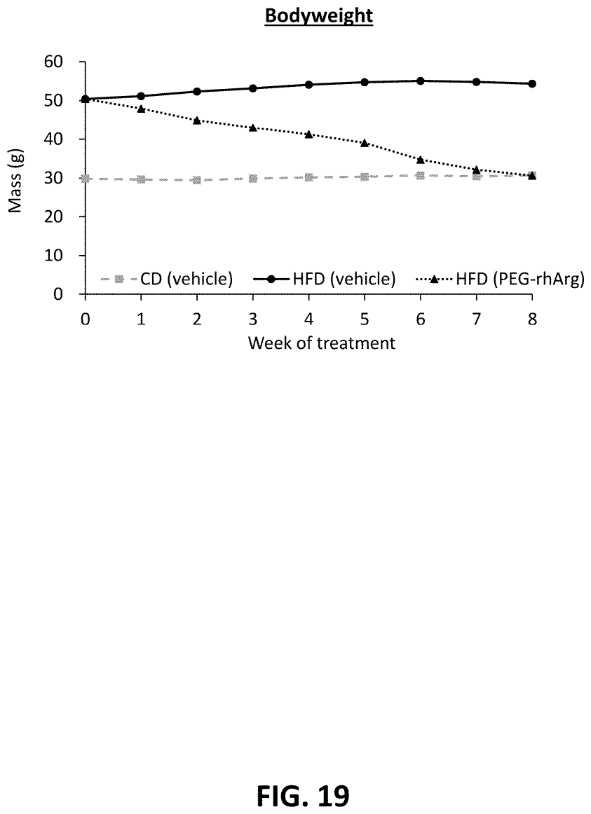

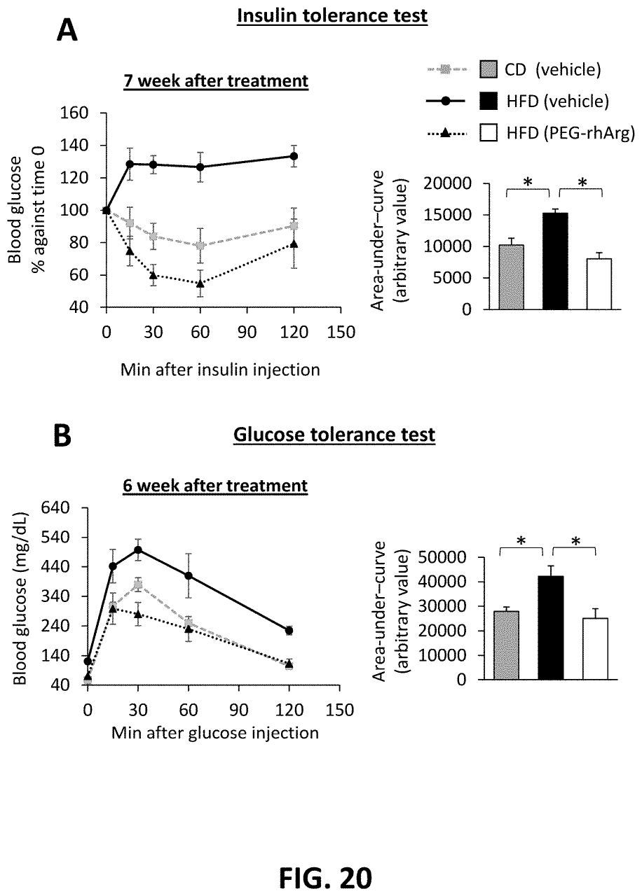

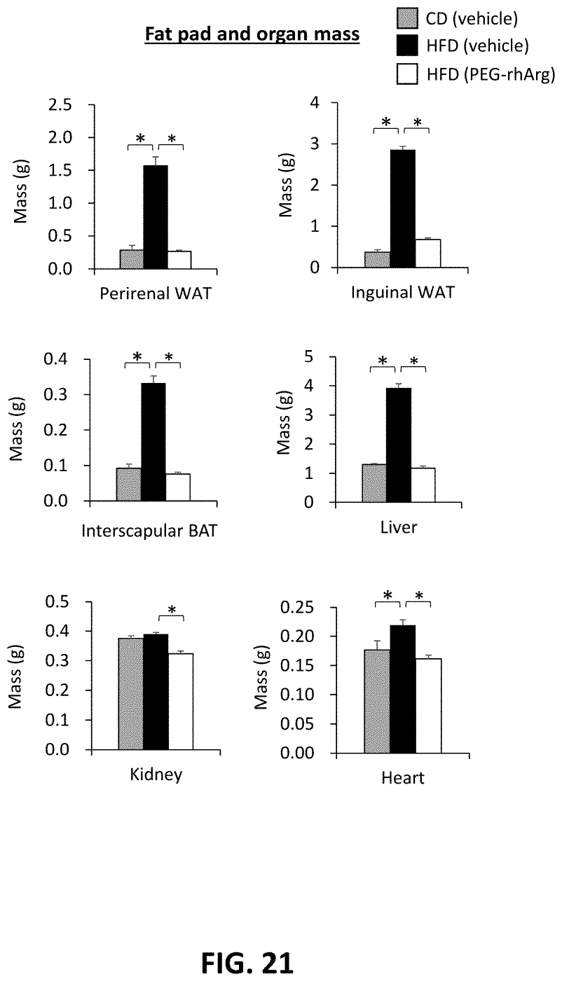

As shown in FIG. 18A, administration of 250 U PEGylated His-rhArg (SEQ ID NO: 101) via i.p. injection once a week for 8 weeks to C57BL/6J male mice with pre-existing obesity induced by feeding a HFD from 5-week of age for 12 weeks [HFD (PEG-rhArg) group], could induce repetitive 7-day intermittent fasting cycles, composed of fasting and refeeding period (FIG. 18B), with a reduction of total food intake per week of about 28% less than vehicle-treated DIO mice [HFD (vehicle) group]. Concomitant with the induction of intermittent fasting, HFD (PEG-rhArg) group of mice had substantial weight loss from 50 g to 30 g within 8 weeks of treatment (FIG. 19). They showed marked increase in insulin sensitivity (FIG. 20A) and significantly improved glucose tolerance (FIG. 20B). At the end of the treatment period, the mass of fat pad of perirenal and inguinal WAT, and interscapular BAT, and the liver mass was dramatically reduced to a weight similar to that of the vehicle-treated lean control mice fed a chow diet [CD (vehicle) group] (FIG. 21). The kidney and heart mass was also significantly reduced.

Together, these findings demonstrate that PEGylated His-rhArg [SEQ ID NO: 101] can induce intermittent fasting, and has anti-adiposity and insulin-sensitizing effects similar to N-ABD094-rhArg [SEQ ID NO: 50]. Arginine deprivation by PEGylated His-rhArg (SEQ ID NO: 101) has therapeutic effects to treat obesity and diseases associated with insulin resistance.

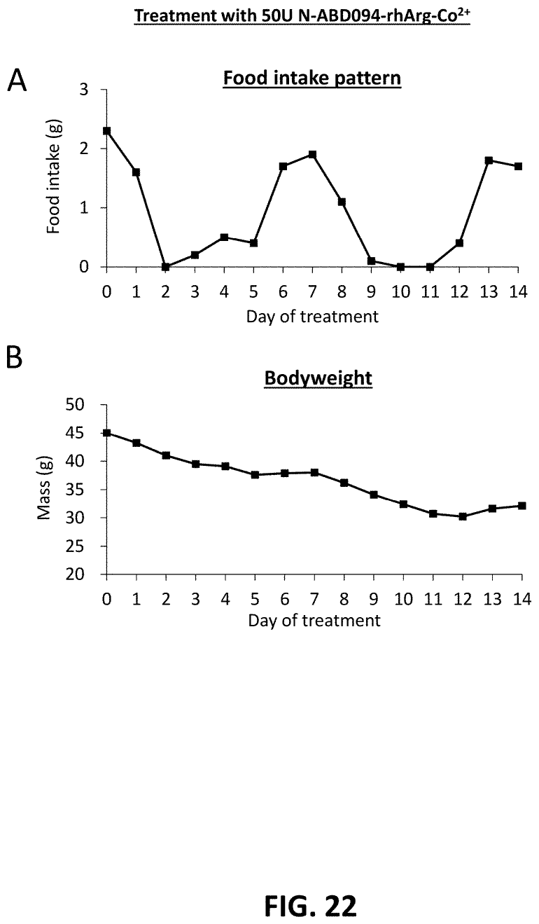

Next, C57BL/6J male mouse with pre-existing diet-induced obesity received i.p. injection of 50 U N-ABD094-rhArg-Co²⁺ [SEQ ID NO: 50 (cobalt substituted)] once a week for 2 weeks while continuously fed a HFD. Mice exhibited a 7-day intermittent fasting cycle with a long fasting period of 4 days followed by refeeding (FIG. 22A). In line with a longer fasting period, there was substantial weight loss from 45 g to 32 g within 2 weeks of treatment (FIG. 22B). These results show that a much lower drug dosage of N-ABD094-rhArg-Co²⁺ can induce a longer fasting period, which implies that substitution of Mn²⁺ with Co²⁺ markedly increase the potency of the ABD-rhArg fusion protein in inducing intermittent fasting cycle and reducing the bodyweight.

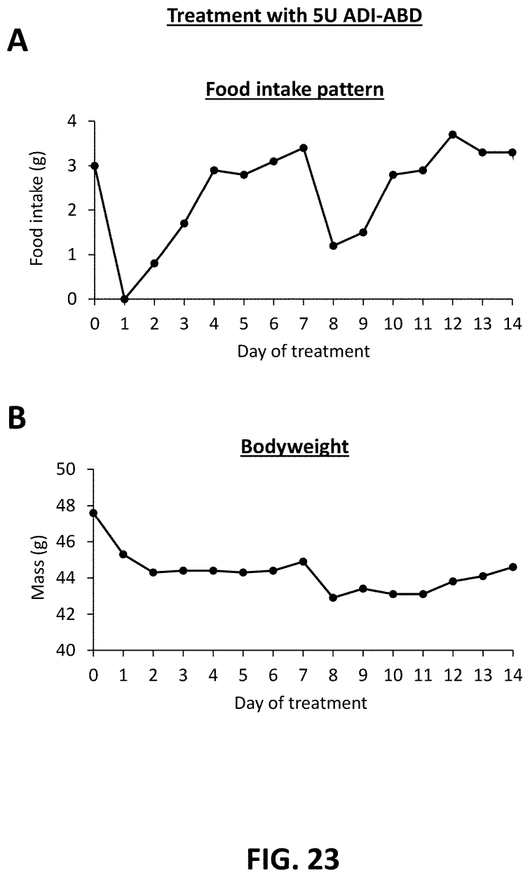

Other than arginase that converts arginine to ornithine and urea, another arginine-depleting enzyme arginine deiminase, which converts arginine to citrulline and ammonia, was examined. C57BL/6J male mouse with pre-existing obesity induced by a HFD was administered via i.p. injection with 5 U ADI-ABD (SEQ ID NO: 107). Food intake decreased to a minimum level after the first day and then gradually increased to the normal level FIG. 23A). The trend of fasting followed by refeeding is similar but not identical to that of N-ABD094-rhArg (SEQ ID NO: 50), which has minimum levels of food intake at about Day 2 to Day 3 instead of Day 1. For the body weight, in the first cycle, it decreased from 47 g to about 44 g in 2 days and maintained at this level during refeeding phase (FIG. 23B).

Together, these findings support that induction of repetitive cycles of intermittent fasting can be achieved by arginine depleting agents other than N-ABD094-rhArg [SEQ ID NO: 50]. The ABD-rhArg fusion protein or other forms of arginase with extended half-life (e.g. rhArg-PEG, BCA-PEG or BCA-ABD) or arginine-depleting enzymes (e.g. ADI-PEG, ADI-ABD, ADC-PEG or ADC-ABD) may have similar therapeutic effects, such as treating obesity and diseases associated with insulin resistance.

### Example 7

### The N-ABD094-rhArg (SEQ ID NO: 50) can Prevent the Development of Cognitive Defects in C57BL/6J Male Mice Fed with HFD

Intermittent fasting has been shown to bring many health benefits and has anti-aging effects. It can help prevent and treat a large variety of diseases. For example, intermittent fasting protects against neurodegeneration. It can enhance cognitive performance.

To determine whether N-ABD094-rhArg [SEQ ID NO: 50], which can induce repetitive intermittent fasting cycles, has beneficial effects on cognitive performance, the Barnes maze test was conducted over ten days to evaluate spatial learning and memory function. The test was performed on a flat circular platform (100 cm in diameter, 1.5 cm thick, elevated 40 cm above the floor) with twenty evenly-spaced holes (7 cm in diameter) distributed around the circumference, and an escape box was placed under one of the holes. Prior to experimentation, each mouse was guided to the specific hole that was positioned over the escape box and left for 180 seconds. Subsequently, each mouse was trained to find the escape hole by placing the mouse in the center of the platform under a small black chamber for three seconds, the chamber was then lifted, and mouse was allowed to find the escape hole within 180 seconds. The platform was brightly illuminated and cooled using a desk fan as adverse stimuli. If the mouse was unable to find the target hole in the allowed time, it was guided to the escape box. Two trials were performed each day for four consecutive days as training period, after which the short- and long-term spatial memory of the mouse was probed on Day 5 and Day 10 respectively, by removing the escape box and allowing them to explore the platform for a full 180 seconds. Each trial was recorded directly over the platform. For each trial on Day 1 to 4, the time required to find the escape hole and exit the platform, and the type of exploratory pattern (random, serial, or direct) in exploring were recorded. For each probe trial on Day 5 and 10, the number of times the mice investigated each of the twenty holes was recorded over 180 seconds, with an instance of investigation defined as the localization of the snout inside a given hole.

Spatial memory loss is a prominent feature in rodent models of type 2 diabetes and obesity [Boitard et al., 2014; Underwood and Thompson, 2016]. To investigate whether arginase treatment could prevent spatial memory loss in mice fed a HFD, DIO C57BL/6J male mice were subjected to assessment of spatial memory by Barnes maze test at 43 weeks after receiving weekly administration of N-ABD094-rhArg (SEQ ID NO: 50) starting from 17-week old.

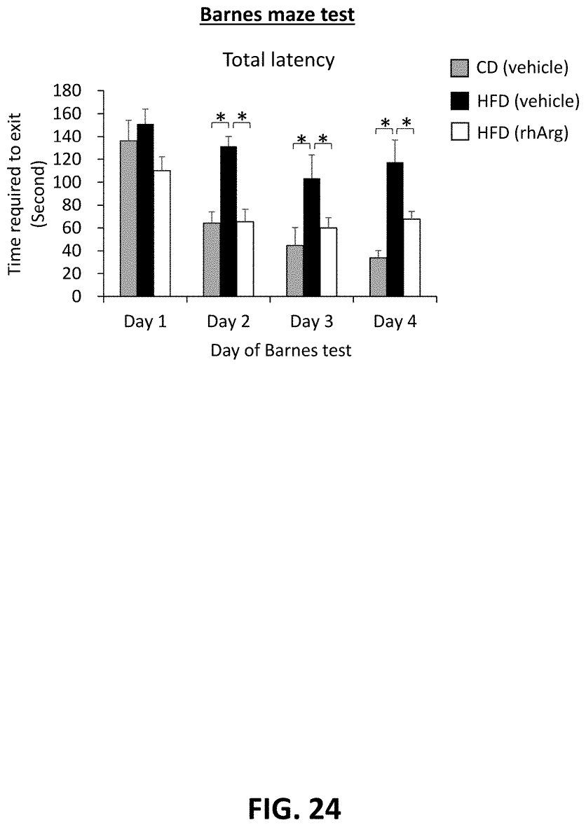

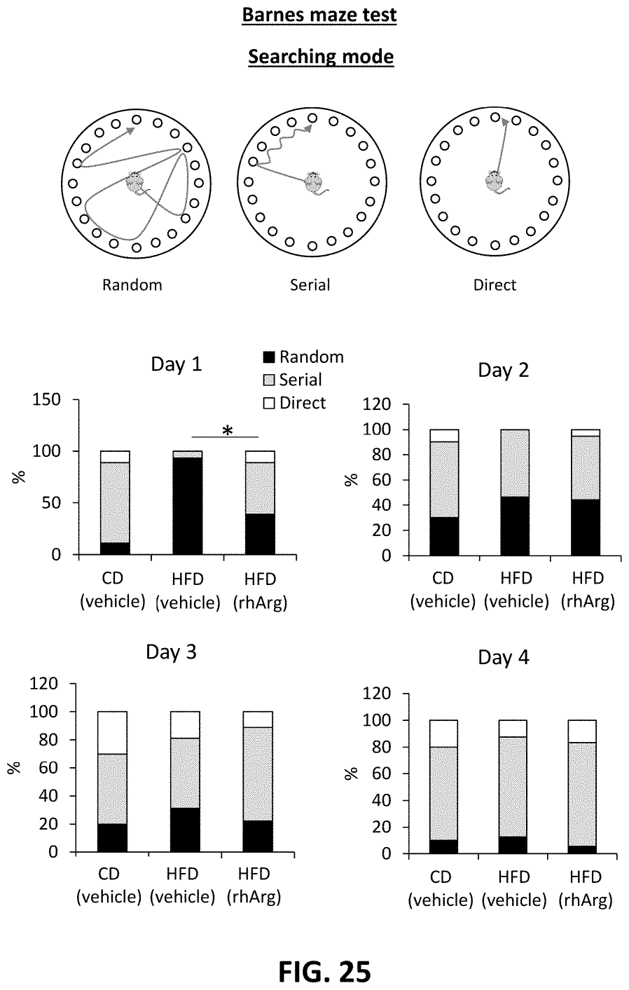

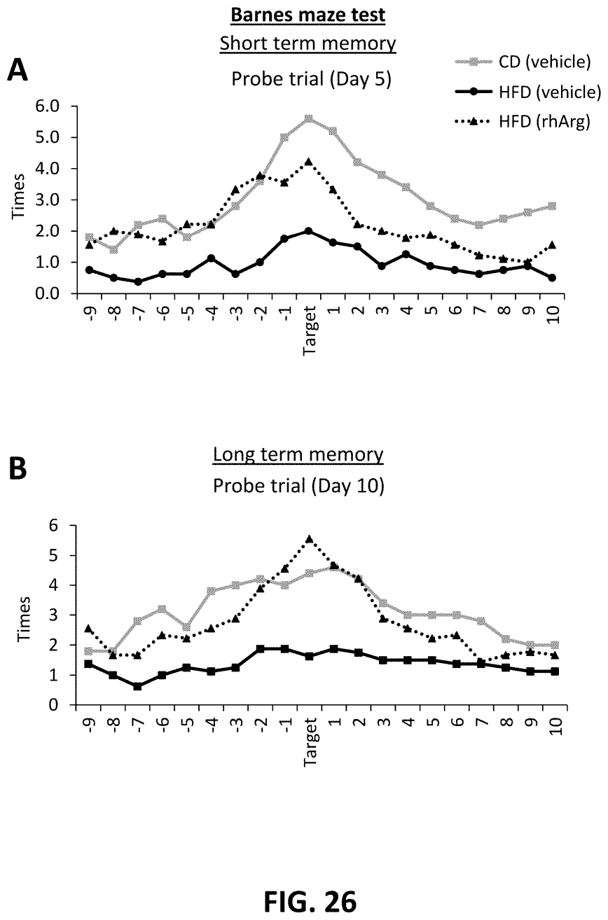

Starting from Day 2 of the training phase, both age-matched vehicle-treated mice fed a chow diet [CD (vehicle) group] and HFD-fed mice treated with N-ABD094-rhArg (SEQ ID NO: 50) [HFD (rhArg) group] could exit the platform significantly earlier than HFD-fed mice treated with vehicle [HFD (vehicle) group] (FIG. 24). At this time, mice from all groups exhibited similar exploratory patterns despite the finding that 90% of mice of HFD (vehicle) group used a random-type exploratory pattern on Day 1 compared to the other two groups, which predominately used a serial-type exploratory pattern (FIG. 25). When short-term memory of the mice was probed on Day 5, mice of CD (vehicle) and HFD (rhArg) groups explored the holes nearest to the target in increasing frequency with the target hole being the most-explored hole, whereas HFD (vehicle) group of mice explored all holes in similar frequency (FIG. 26A). Such trends were maintained on Day 10 when the long-term memory of the mice was probed (FIG. 26B).

Together, these findings support that treatment of ABD-rhArg fusion protein or other forms of arginase (e.g., rhArg-PEG, BCA-PEG or BCA-ABD) or arginine-depleting enzyme (e.g., ADI-PEG, ADI-ABD, ADC-PEG or ADC-ABD) may have therapeutic effects to prevent or improve cognitive defects.

### Example 8

### The N-ABD094-rhArg (SEQ ID NO: 50) can Rejuvenate Neuromuscular Strength and Coordination of C57BL/6J Male Mice Fed with HFD

To evaluate balance, neuromuscular strength and coordination of mice, DIO C57BL/6J male mice fed a HFD were subjected to inverted grid hanging test and rotarod test at 42 week and 30 week respectively after receiving weekly administration of N-ABD094-rhArg (SEQ ID NO: 50) starting from 17-week old.

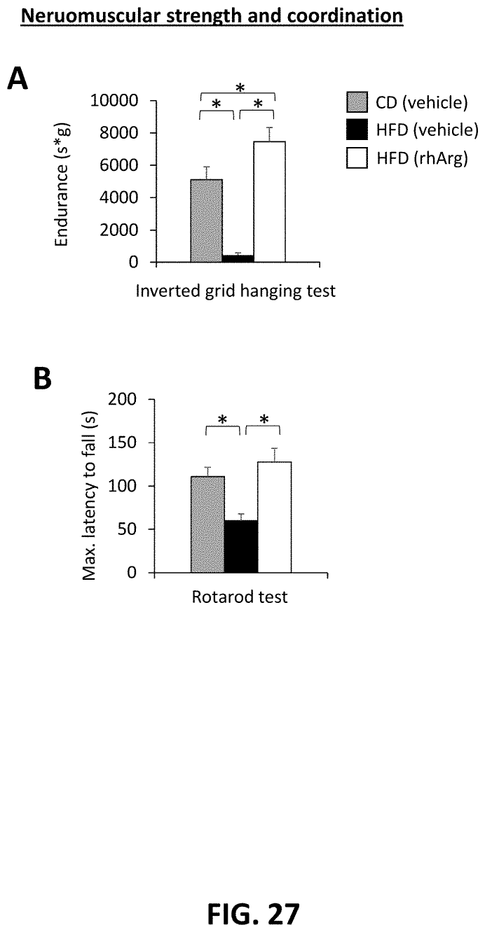

The four limb inverted grid hanging test uses a wire grid set up to non-invasively measure the ability of mice to exhibit sustained limb tension to oppose their gravitational force. The time (latency) it took the mouse to fall off the grid was recorded. Results showed that vehicle-treated mice with long-term HFD feeding [HFD (vehicle) group] exhibited severe reduced neuromuscular strength with a 90% decrease in the endurance when compared with vehicle-treated control mice fed a chow diet [CD (vehicle) group]. In contrast, HFD-fed mice receiving weekly treatment of N-ABD094-rhArg (SEQ ID NO: 50) [HFD (rhArg) group] for 42 weeks exhibited significantly improved neuromuscular performance, with the endurance even higher than mice in CD (vehicle) group (FIG. 27A).

Rotarod test is used to assess neuromuscular coordination and balance in rodents. Mice have to keep their balance on a rotating rod with accelerating mode from 5 to 40 rpm in 300 sec. The time (latency) it took the mouse to fall off the rod was recorded. Results showed that vehicle-treated mice with long term HFD feeding [HFD (vehicle) group] exhibited a shorter latency on the rotating rod than vehicle-treated control mice fed a chow diet [CD (vehicle) group]. In contrast, HFD-fed mice receiving weekly treatment of N-ABD094-rhArg (SEQ ID NO: 50) [HFD (rhArg) group] for 30 weeks showed significantly improved performance in the rotarod test with the latency similar to mice in CD (vehicle) group (FIG. 18).

Together, these findings support that treatment of ABD-rhArg fusion protein or other forms of arginase (e.g., rhArg-PEG, BCA-PEG or BCA-ABD) or arginine-depleting enzymes (e.g., ADI-PEG, ADI-ABD, ADC-PEG or ADC-ABD) may have therapeutic effects to improve neuromuscular strength and coordination.

### Example 9

### The N-ABD094-rhArg (SEQ ID NO: 50) can Prevent Liver Cancer in C57BL/6J Male Mice Fed with HFD

Intermittent fasting has been shown to have anti-aging effects and can protect against cancer, which is one of the biomarkers of aging.

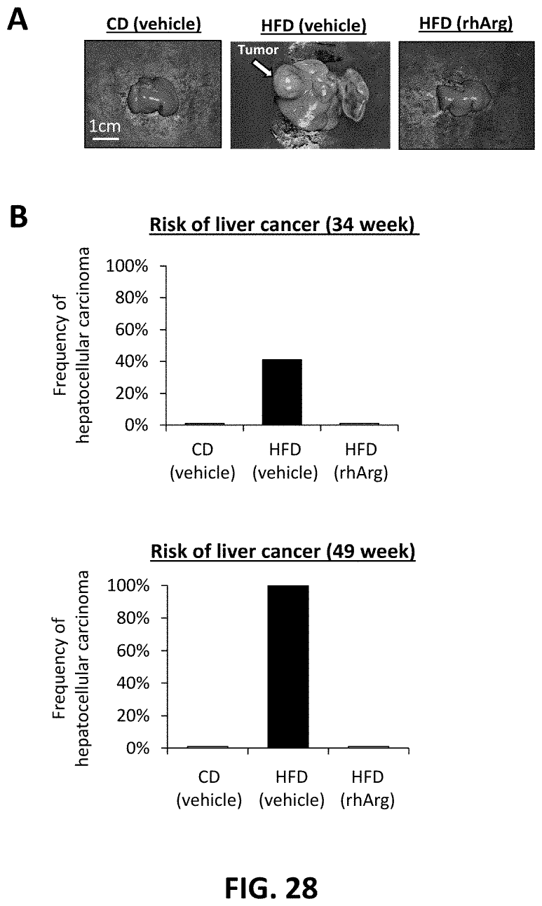

Development of neoplasm in the liver is one of the major characteristics of aging in C57BL/6J male mice with long-term feeding on a HFD, which may be related to the development of severe non-alcoholic fatty liver disease. Treatment with N-ABD094-rhArg [SEQ ID NO: 50] can effectively reverts hepatic steatosis and lower serum concentrations of liver damage biomakers ALT and AST (FIG. 7). Autopsy performed at the end of experiment confirmed the presence of hepatocellular carcinoma (FIG. 28A) in around 40% of C57BL/6J male mice that had been fed on a HFD for 46 weeks (12+34 weeks) starting from 5-week old [HFD (vehicle) group in FIG. 1] (FIG. 28B). The incidence rate even increased to 100% in mice fed on the HFD for 61 weeks (12+49 weeks) [HFD (vehicle) group in FIG. 3] (FIG. 28C). In contrast, in the DIO C57 BL/6J male mice that received weekly injection of N-ABD094-rhArg for 34 weeks [HFD (rhArg) group in FIG. 1] and 49 weeks [HFD (rhArg) group in FIG. 3] starting from 17-week of age, none of the mice developed hepatocellular carcinoma. These findings support that rhArg has potent effects on prevention of liver cancer.

### Example 10

### The N-ABD094-rhArg (SEQ ID NO: 50) Induces Intermittent Fasting and Reduces Bodyweight in Obese ICR Female Mice Fed with HFD

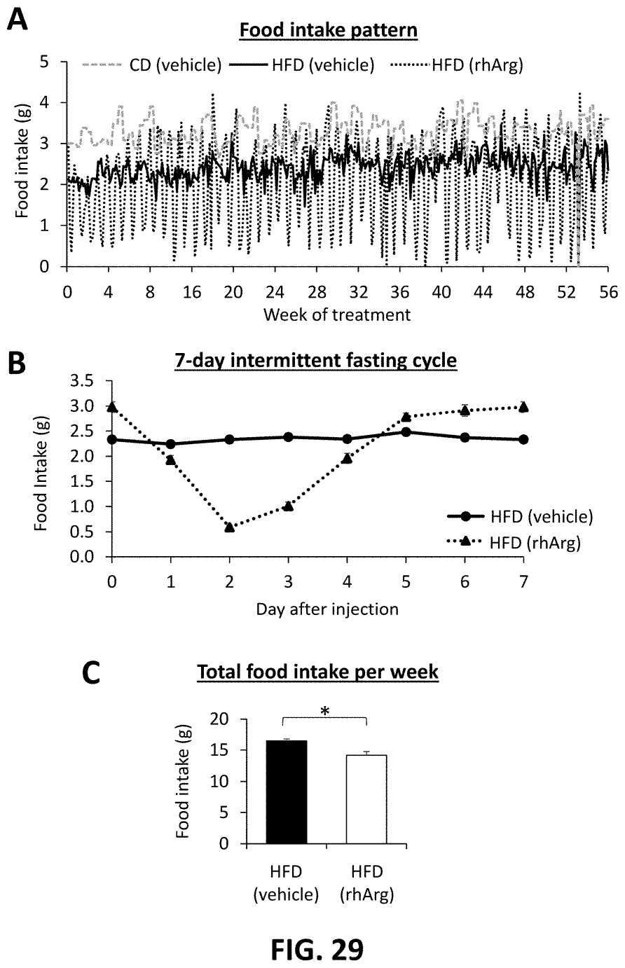

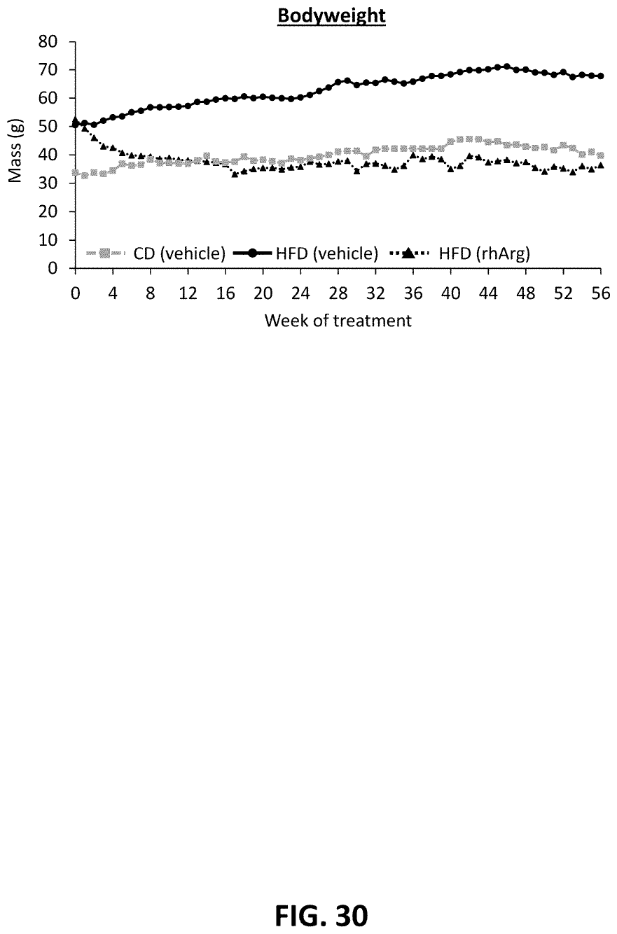

After confirming the effect of N-ABD094-rhArg (SEQ ID NO: 50) in inducing intermittent fasting with concomitant reduction in bodyweight in male C57BL/6J mice, the effect of N-ABD094-rhArg (SEQ ID NO: 50) on female mice was studied to determine if there could be any sex difference. We found that, similar to the results of other reports, C57BL/6J female mice is not as susceptible as C57BL/6J male mice to diet-induced obesity. Thus, we employed ICR female mice, which developed obesity with a bodyweight of around 50 g after 12 weeks of feeding with a HFD starting from 5 weeks of age. FIG. 29 shows the results of administration, via i.p. injection, of 1200 U of N-ABD094-rhArg (SEQ ID NO: 50) once a week to diet-induced obese ICR female mice for 56 weeks, which continued to consume a HFD [HFD (rhArg) group]. Results of daily monitoring of food intake showed that a 7-day intermittent fasting cycle (FIG. 29A), consisting of period of fasting followed by refeeding (FIG. 29B), with a pattern similar to that in C57BL/6J male mice (FIG. 1B), was repeatedly occurring in ICR female mice throughout the 56 weeks of treatment period. Despite a reduction of total food intake per week by an average of 14% (FIG. 29C) in comparison to vehicle-treated ICR female mice fed a HFD [HFD (vehicle) group], within 9-10 weeks, the bodyweight decreased significantly by 30% to a level similar to the bodyweight of age-matched lean control ICR female mice fed a chow diet [CD (vehicle) group] (FIG. 30). The bodyweight of mice in HFD (rhArg) group could be maintained at that level for the rest of the treatment period despite continued consumption on a HFD. In contrast, mice in both HFD (vehicle) and CD (vehicle) groups showed gradual bodyweight gain from 50 g to 70 g and from 32 g to 45 g respectively over the 56-week of treatment period.

Together, these findings support that treatment of ABD-rhArg fusion protein or other forms of arginase (e.g., rhArg-PEG, BCA-PEG or BCA-ABD) or arginine-depleting enzymes (e.g., ADI-PEG, ADI-ABD, ADC-PEG or ADC-ABD) can induce intermittent fasting and have anti-obesity effects in both sexes.

### Example 11

### The N-ABD094-rhArg (SEQ ID NO: 50) Prevents Age-Related Diseases in ICR Female Mice Fed a HFD

Increased adiposity, insulin resistance, impaired glucose tolerance, decline in neuromuscular strength and motor coordination, and increased risk of cancer are common age-related changes that are further accelerated by obesity. Studies were conducted to determine whether there could be any improvements in these parameters in concomitant with induction of intermittent fasting in female mice fed a HFD via weekly injection of N-ABD094-rhArg (SEQ ID No: 50) for 56 weeks.

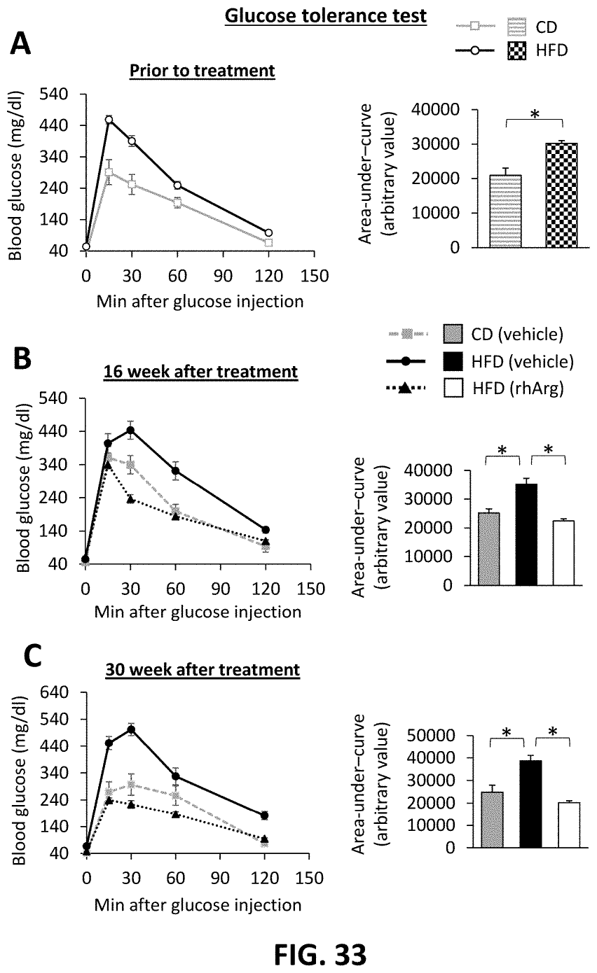

In line with the results of reduction in bodyweight (FIG. 30), at the end of 56 weeks of treatment period, the fat pad mass of perirenal and visceral WAT, interscapular BAT, and liver, kidney and heart mass in HFD-fed mice treated with N-ABD094-rhArg (SEQ ID NO: 50) [HFD (rhArg) group] was significantly less than that of HFD-fed mice treated with vehicle [HFD (vehicle) group] (FIG. 31). Furthermore, similar to the effect on C57BL/6J male mice, weekly treatment with N-ABD094-rhArg (SEQ ID NO: 50) could reverse pre-existing insulin resistance (FIG. 32A) and impaired glucose tolerance (FIG. 33A). In fact, mice in HFD (rhArg) group showed better insulin sensitivity than age-matched mice in CD (vehicle) group in insulin tolerance test conducted at 15 weeks (FIG. 32B) and 31 weeks (FIG. 32C). Improved glucose tolerance was also confirmed by glucose tolerance test conducted at 16 weeks (FIG. 33B) and 30 weeks (FIG. 33C) after receiving rhArg treatment.

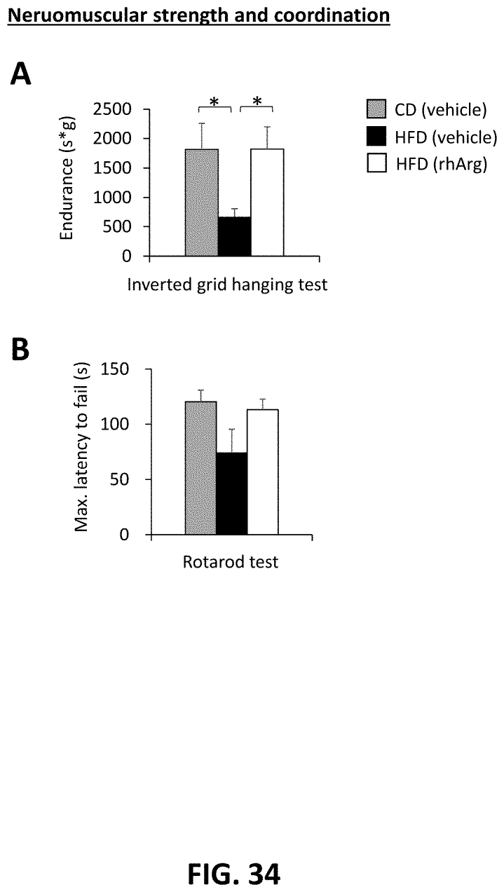

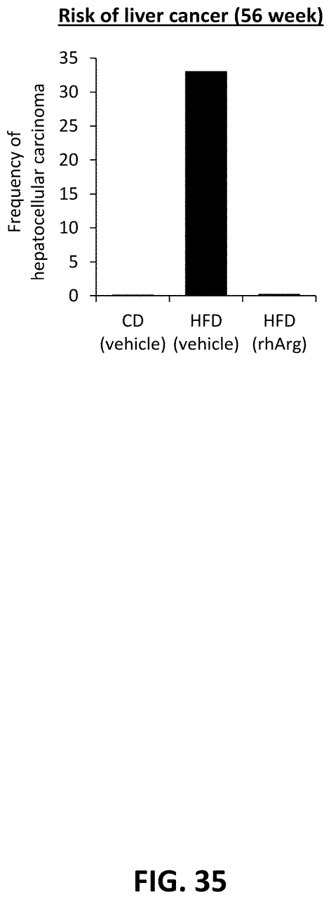

Furthermore, the performance of HFD (rhArg) group of mice in the inverted grid hanging test (FIG. 34A) and rotarod test (FIG. 34B) conducted respectively at 54 weeks and 55 weeks of treatment period was significantly better than mice in HFD (vehicle) group, and was comparable with age-matched CD (vehicle) group, showing that treatment with N-ABD094-rhArg (SEQ ID NO: 50) could prevent decline in neuromuscular strength and coordination. Furthermore, while over 30% of mice in HFD (vehicle) group developed hepatocellular carcinoma (FIG. 35), none of the mice in HFD (rhArg) group developed hepatocellular carcinoma, thus demonstrating the potent anti-cancer effect of N-ABD094-rhArg (SEQ ID NO: 50).

Together, these findings support that treatment of ABD-rhArg fusion protein or other forms of arginase (e.g., rhArg-PEG, BCA-PEG or BCA-ABD) or arginine-depleting enzymes (e.g., ADI-PEG, ADI-ABD, ADC-PEG or ADC-ABD) can induce intermittent fasting, with concomitant prevention of various aging-associated diseases in both genders.

### Example 12

### The N-ABD094-rhArg (SEQ ID NO: 50) Induces Intermittent Fasting and Improves Metabolic Health in Middle-Aged Male C57BL/6J Male Mice Fed a HFD

In previous studies, HFD-induced obese male (Example 1) and female mice (Example 10) received treatment of N-ABD094-rhArg (SEQ ID NO: 50) starting from about 4-5 months old, which is equivalent to about mid-twenties in humans. To determine the susceptibility of middle-aged subjects to arginine depletion, obese C57BL/6J male mice, which had been fed a HFD since 5 weeks old, received weekly injection of N-ABD094-rhArg (SEQ ID NO: 50) for 25 weeks starting from around 16 months of age [HFD Old (rhArg) group], which is equivalent to mid-fifties in humans.

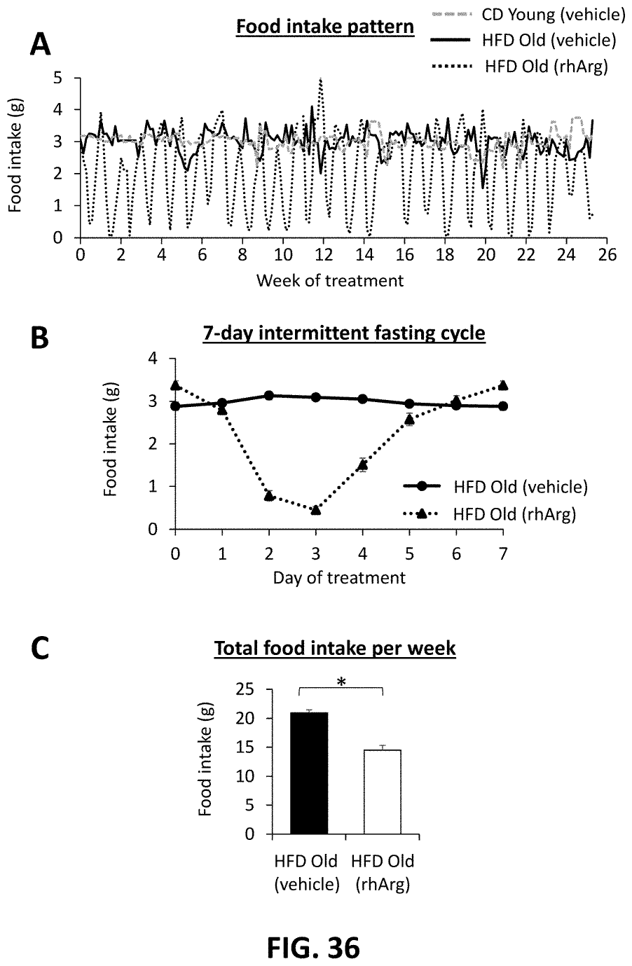

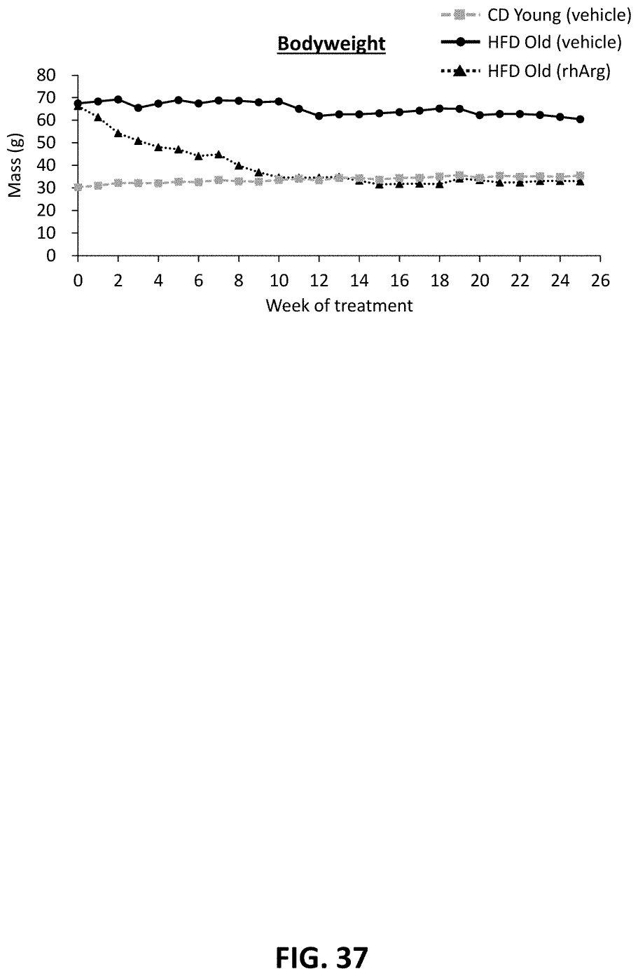

As shown in FIG. 37, the mean bodyweight of HFD-fed mice at 16-month old was about 68 g prior to treatment. Once weekly injection of N-ABD094-rhArg (SEQ ID NO: 50) could induce repetitive 7-day intermittent fasting cycles (FIG. 36A), composed of fasting and refeeding period (FIG. 36B). Overall, there was 31% reduction of food intake per week in comparison to HFD-fed mice treated with vehicle [HFD Old (vehicle) group]. Such a pattern is very similar to the response of young mice to treatment with N-ABD094-rhArg (SEQ ID NO: 50) (FIG. 1B, 3B) and other arginine-depleting agents (FIG. 18B).

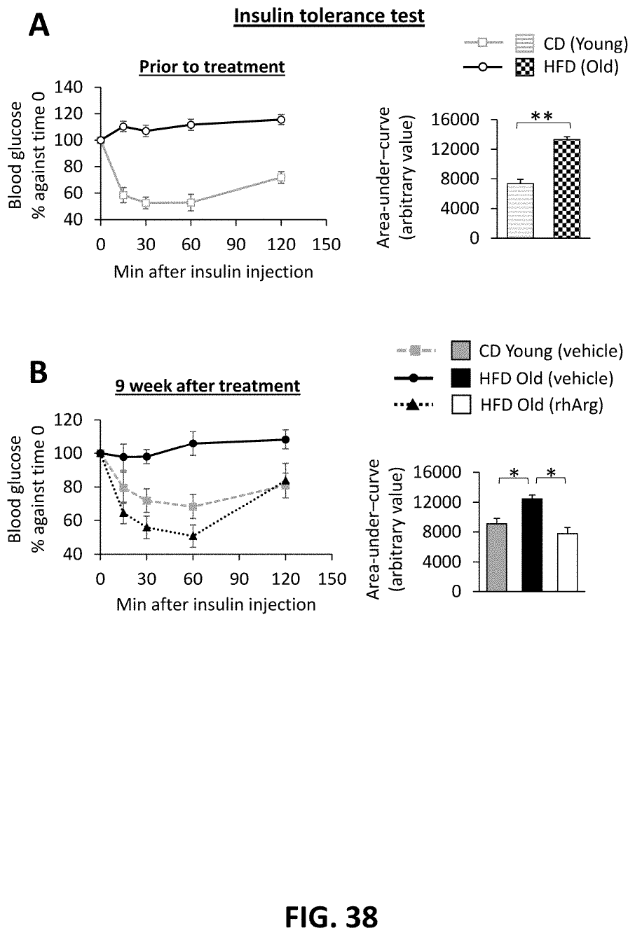

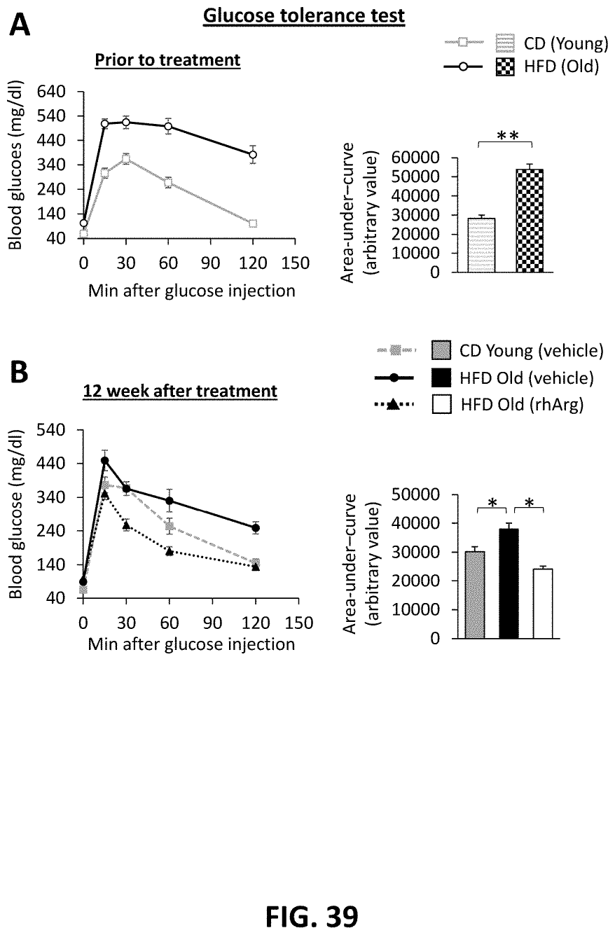

Concomitant with the induction of repetitive cycles of intermittent fasting, mice in HFD Old (rhArg) group showed progressive reduction in bodyweight, such that the bodyweight markedly dropped by 50% within 9-10 months, and was well-maintained at that level throughout the remaining treatment period (FIG. 37). Other than inducing substantial bodyweight loss, N-ABD094-rhArg (SEQ ID NO: 50) treatment could markedly reverse pre-existing insulin resistance (FIG. 38A) and impaired glucose tolerance (FIG. 39A), and increased insulin sensitivity (FIG. 38B) and glucose tolerance (FIG. 39B) in mice of HFD Old (rhArg) group to a level similar, if not better, than vehicle-treated mice that were around 7-8 month old and fed a chow diet [CD Young (vehicle)].

Together, these findings support that treatment of ABD-rhArg fusion protein or other forms of arginase (e.g., rhArg-PEG, BCA-PEG or BCA-ABD) or arginine-depleting enzymes (e.g., ADI-PEG, ADI-ABD, ADC-PEG or ADC-ABD) can induce intermittent fasting in diet-induced obese mice disregard of age, and improve metabolic health.

### Example 13

### The N-ABD094-rhArg (SEQ ID NO: 50) Induces Intermittent Fasting and Weight Loss in Male C57BL/6J Male Mice Fed an Ordinary Chow

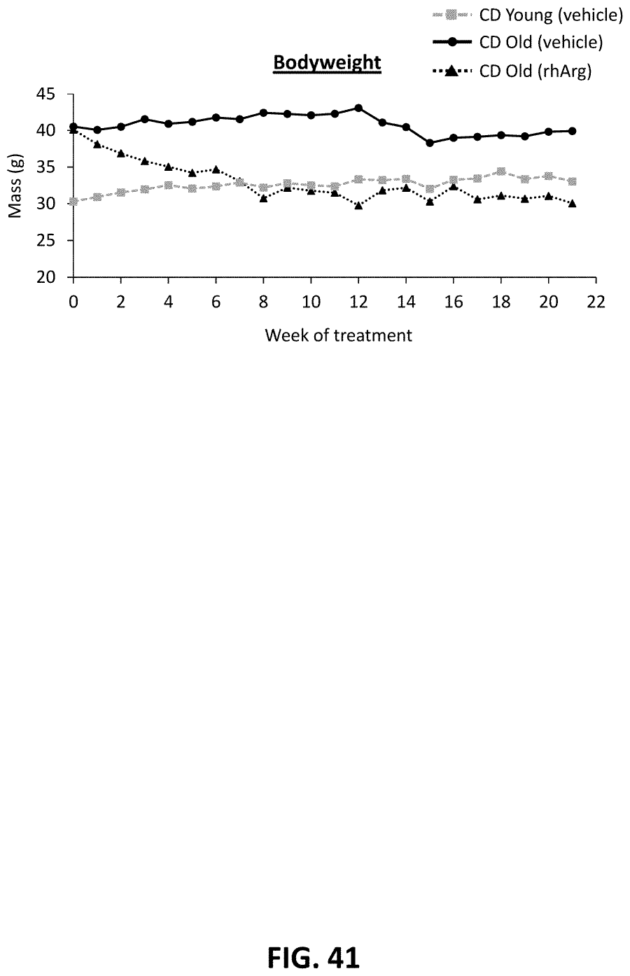

Increase in bodyweight and adiposity are common age-related changes. For instance, C57BL/6J male mice fed an ordinary chow had a bodyweight of 30 g at 5 months old (equivalent to mid-twenties in humans), which progressively increases to 40 g at 18 months old (equivalent to mid-fifties in humans) (FIG. 41). To determine whether N-ABD094-rhArg (SEQ ID NO: 50) can have metabolic benefits on age-related adiposity, old C57BL/6J male mice at 18 months of age fed an ordinary chow were injected once weekly with N-ABD094-rhArg (SEQ ID NO: 50) [CD Old (rhArg) group] or vehicle [CD Old (vehicle) group] for 5 months. C57BL/6J male mice at 5 months old served as the young control [CD Young (vehicle)] (FIG. 41).

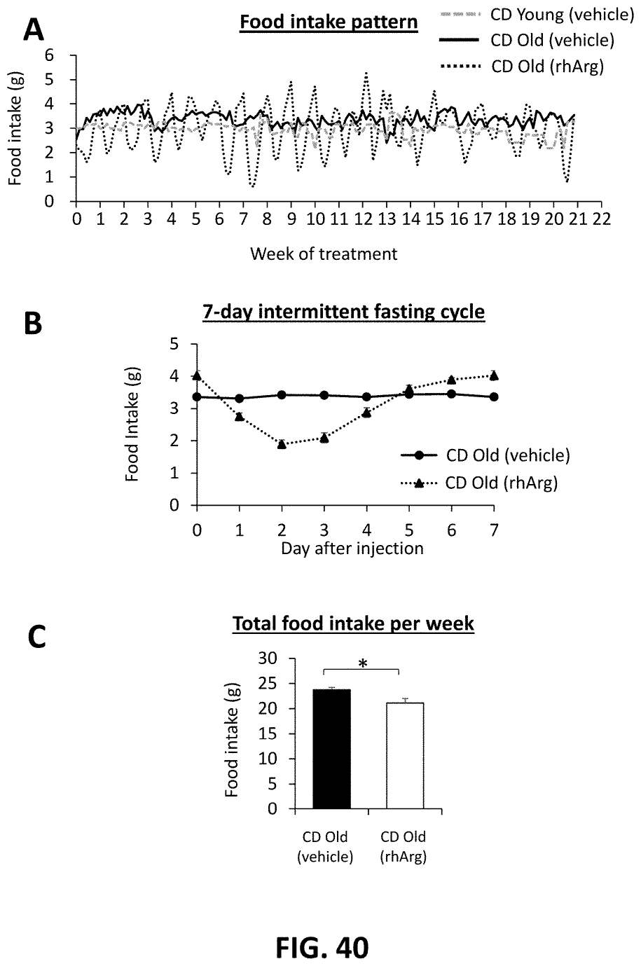

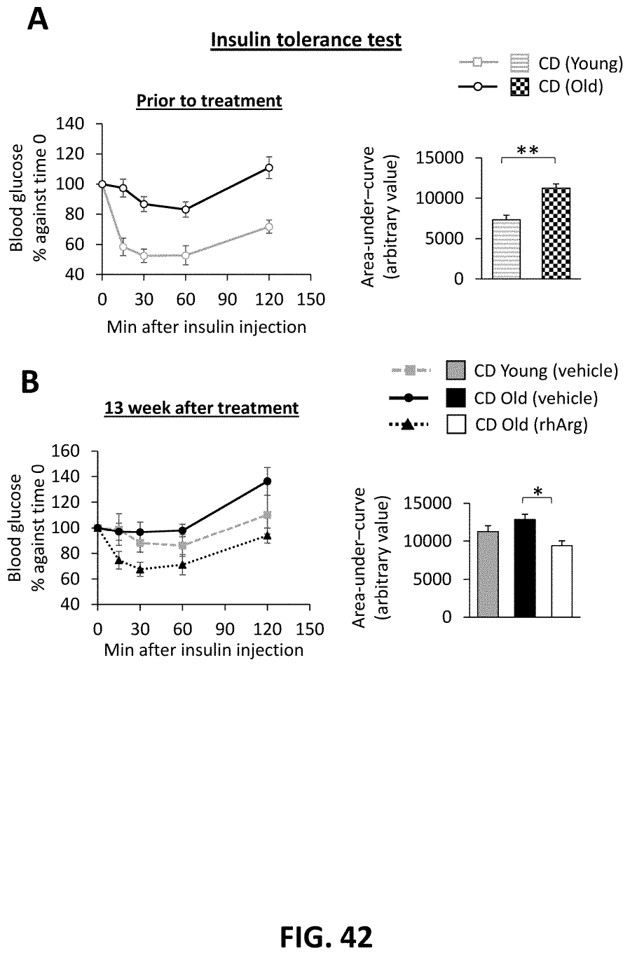

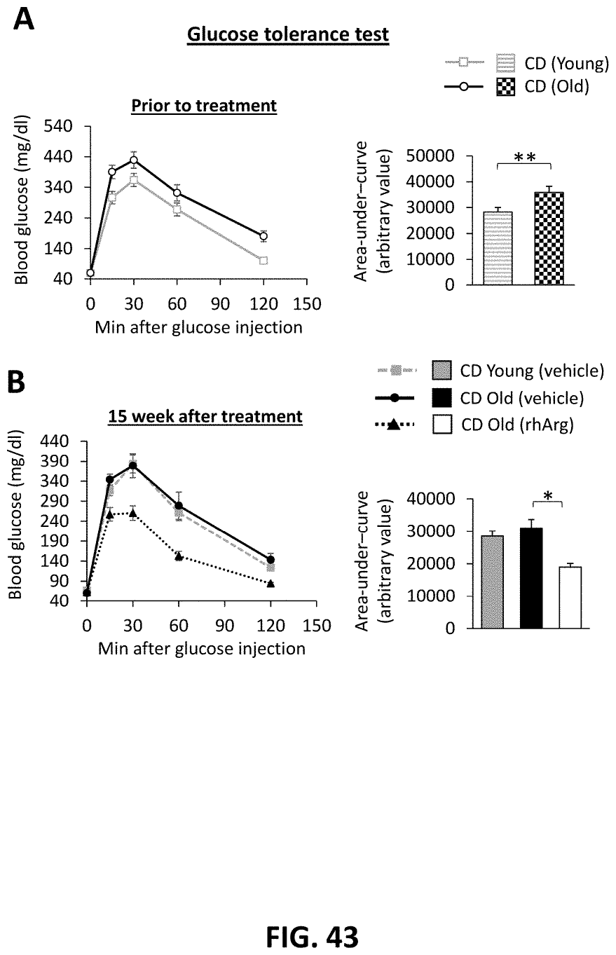

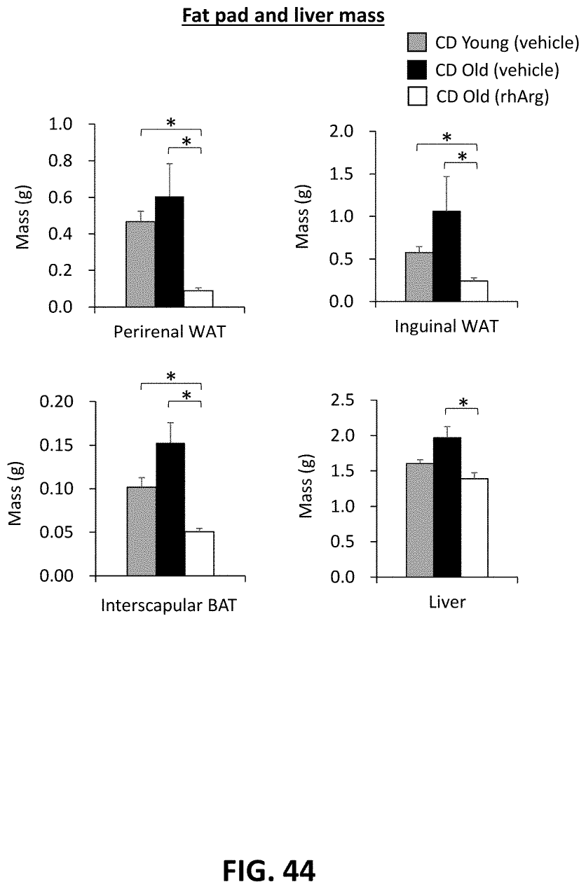

As shown in FIG. 40A, once weekly injection of N-ABD094-rhArg (SEQ ID NO: 50) could induce repetitive 7-day intermittent fasting cycles with period of fasting and refeeding (FIG. 40B) in CD Old (rhArg) group of mice, and the total food intake per week was reduced by 11% in comparison to mice in CD Old (vehicle) group (FIG. 40C). While the bodyweight of mice in CD Young (vehicle) group progressively increased from 30 g at 5-month old to 35 g at 10-month old, the bodyweight of mice in CD Old (rhArg) group reduced to 30 g within 6 weeks of N-ABD094-rhArg (SEQ ID NO: 50) treatment and was well-maintained at that level throughout the rest of the treatment period. In line with a decrease in bodyweight, there was significant reduction in the fat pad mass of perirenal and inguinal WAT, interscapular BAT and liver mass (FIG. 44) to such an extent that the mass of some of these organs was even lower than that of mice in CD Young (vehicle) group. Other than reducing adiposity, N-ABD094-rhArg (SEQ ID NO: 50) treatment could also significantly improve insulin sensitivity (FIGS. 42A and B) and glucose tolerance (FIGS. 43A and B) in CD Old (rhArg) mice to a level better than mice in CD Young (vehicle) group as this group of mice was increasing in age.

Together, these findings support that treatment of ABD-rhArg fusion protein or other forms of arginase (e.g., rhArg-PEG, BCA-PEG or BCA-ABD) or arginine-depleting enzymes (e.g., ADI-PEG, ADI-ABD, ADC-PEG or ADC-ABD) can induce intermittent fasting in mice fed an ordinary chow diet and improve metabolic health.

### Example 14

### The N-ABD094-rhArg (SEQ ID NO: 50) Increases the Lifespan of Aged C57BL/6J Male Mice Fed an Ordinary Chow

Specific autophagy upregulation in Caenorhabditis elegans and Drosophila extends lifespan, and drugs that induce autophagy, such as rapamycin, promote longevity in rodents (Hansen et al., Nature Reviews Molecular Cell Biology. 2018; 19:579-593). For C57BL/6J mice, over 24 months of age can be considered “very old” and survivorship drops off markedly (https://www.jax.org/news-and-insights/jax-blog/2017/november/when-are-mice-considered-old). To determine whether N-ABD094-rhArg (SEQ ID NO: 50), which induces systemic upregulation of autophagy, can increase longevity, very old C57BL/6J male mice at 25 months of age (equivalent to seventy in humans) fed an ordinary chow diet were separated into 2 groups. One group was treated once weekly with 600 U N-ABD094-rhArg (SEQ ID NO: 50) [CD Very Old (rhArg) group] for 30 weeks while another group was treated with vehicle [CD Very Old (vehicle) group]. A group of C57BL/6J male mice at 7 months of age (equivalent to early thirties in humans) treated with vehicle [CD Young (vehicle) group] served as the reference of progressive changes from mature adult to late middle age over the 30-week period.

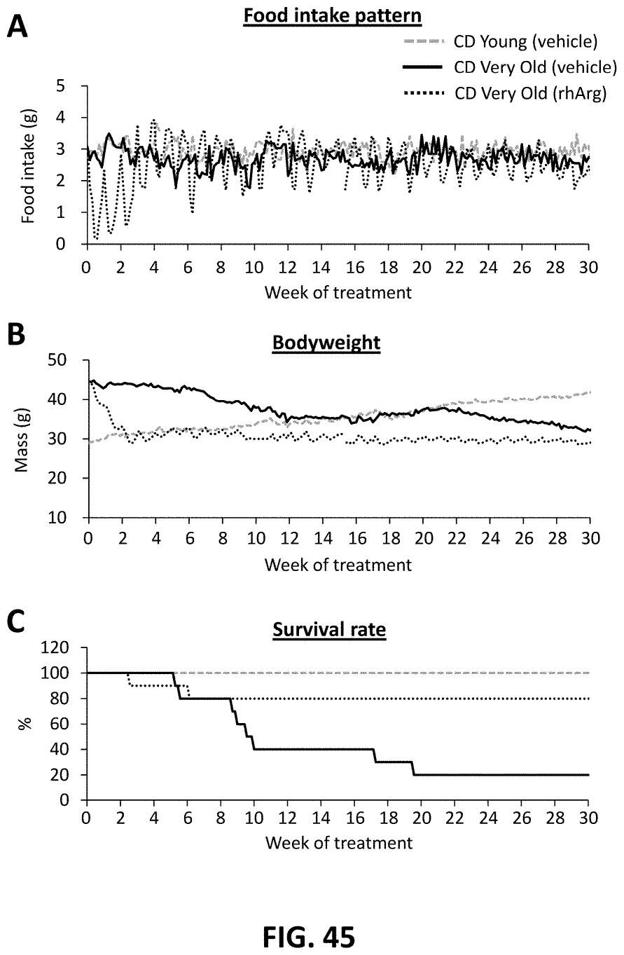

As shown in FIG. 45B, very old mice exhibited age-related obesity with the bodyweight reaching 45 g at 2 years of age. Treatment of very old mice with N-ABD094-rhArg (SEQ ID NO: 50) [CD Very Old (rhArg) group] once a week induced prominent repetitive 7-day intermittent fasting cycles (FIG. 45A), with a concomitant reduction in bodyweight that decreased to 30 g within 3 weeks, which was similar to the bodyweight of mature adult mice at 7-month old [CD Young (vehicle) group]. Mice in the [CD Young (vehicle) group] showed gradual increase in bodyweight from 30 g to 43 g over the 7-month study period as they progressed through middle age. In contrast, for very old mice [CD Very Old (vehicle) group], as they further advanced in age, their bodyweight gradually declined. Moreover, the survival rate dropped to 20% at 30 months of age (end of study) (FIG. 45C). Notably, with weekly injection of N-ABD094-rhArg (SEQ ID NO: 50), the survival rate was maintained at 80%, implicating a prominent increase in lifespan.

These findings support that ABD-rhArg fusion protein or other forms of arginase (e.g., rhArg-PEG, BCA-PEG or BCA-ABD) or arginine-depleting enzymes (e.g., ADI-PEG, ADI-ABD, ADC-PEG or ADC-ABD) can increase longevity of animals.

### Example 15

### The N-ABD094-rhArg (SEQ ID NO: 50) Induces Autophagy (Lipophagy) During the Fasting Phase of the 7-Day Intermittent Fasting Cycle to Break Down Lipids in the Liver of C57BL/6J Mice Fed a HFD

Autophagy is involved in cell growth, survival, development and death. Impaired autophagic flux has been linked to a variety of human pathophysiological processes, including neurodegeneration, cancer, myopathy, cardiovascular and immune-mediated disorders. There is a growing need to identify and quantify the status of autophagic flux in different pathological conditions. Autophagy is a highly dynamic and complex process that is regulated at multiple steps. Autophagic flux can be detected by LC3-II turnover using western blot analysis in the presence and absence of lysosomal degradation inhibitors, chloroquine (CQ). If autophagic flux is occurring, the level of LC3-II will increase in the presence of a lysosomal degradation inhibitor because the transit of LC3-II through the autophagic pathway will be blocked.

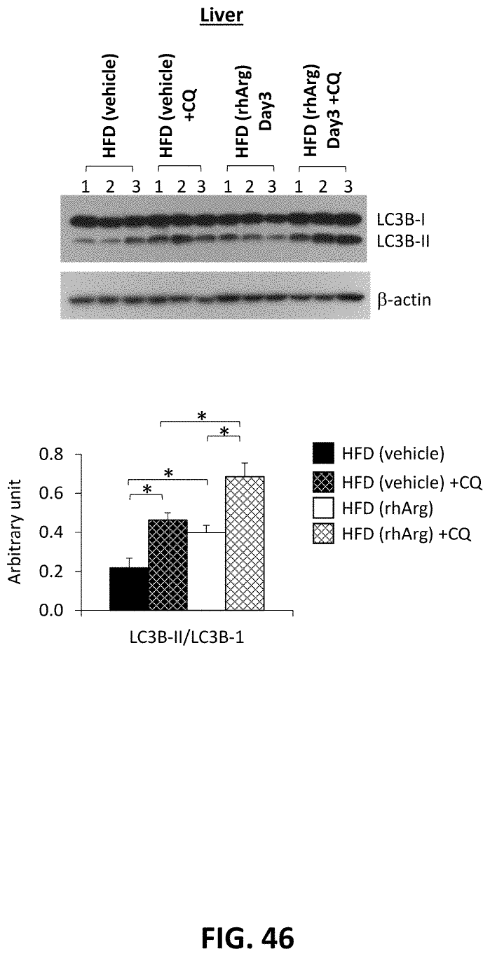

Our results showed that the LC3-II/LC3-I ratio in the liver sample of the N-ABD094-rhArg (SEQ ID NO: 50)-treated group showed dramatic increase upon injection of CQ for 5 hrs (FIG. 46). This indicated that N-ABD094-rhArg treatment (Day 3) induced autophagy in the liver of DIO mice at 4 wk.

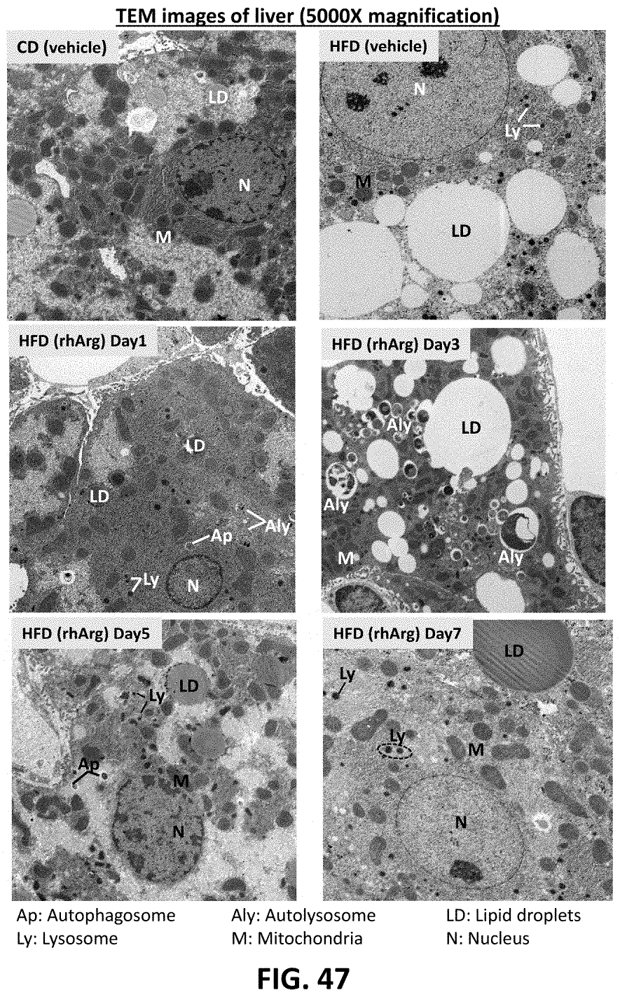

FIG. 47 shows the transmission electron microscopy images of the liver sections of mice fed a chow diet (CD) or HFD, and administered with N-ABD094-rhArg (SEQ ID NO: 50) (rhArg) or saline (vehicle) for 4 weeks. In mice fed with HFD and administered with vehicle [HFD (vehicle) group], accumulation of large lipid droplets and lysosomes was observed in the hepatocyte (liver cell). In mice fed with HFD and administered with N-ABD094-rhArg (SEQ ID NO: 50) [HFD (rhArg) group], at Day 1 of the 7-day intermittent fasting cycle, autophagosomes and autolysosomes were found in the hepatocye. However, no lipophagy was observed at this stage yet. At Day 3 of the 7-day intermittent fasting cycle, extensive lipophagy was observed in the hepatocye. There were plenty of autolysosomes, which were breaking down the lipid content in the hepatocyte. At Day 5 of the 7-day intermittent fasting cycle, autophagosome can still be found in the hepatocye, but autolysosome and lipophagy became rare. This indicated that cyclic activation of lipophagy nearly came to an end. At Day 7 of the 7-day intermittent fasting cycle, there was no autophagosome and no lipophagy was observed. There was lysosome accumulation, which was similar to the HFD (vehicle) group, but the lipid content accumulated in the hepatocyte was reduced. The accumulated lipid content was similar to that observed in mice fed the chow diet [CD (vehicle) group].

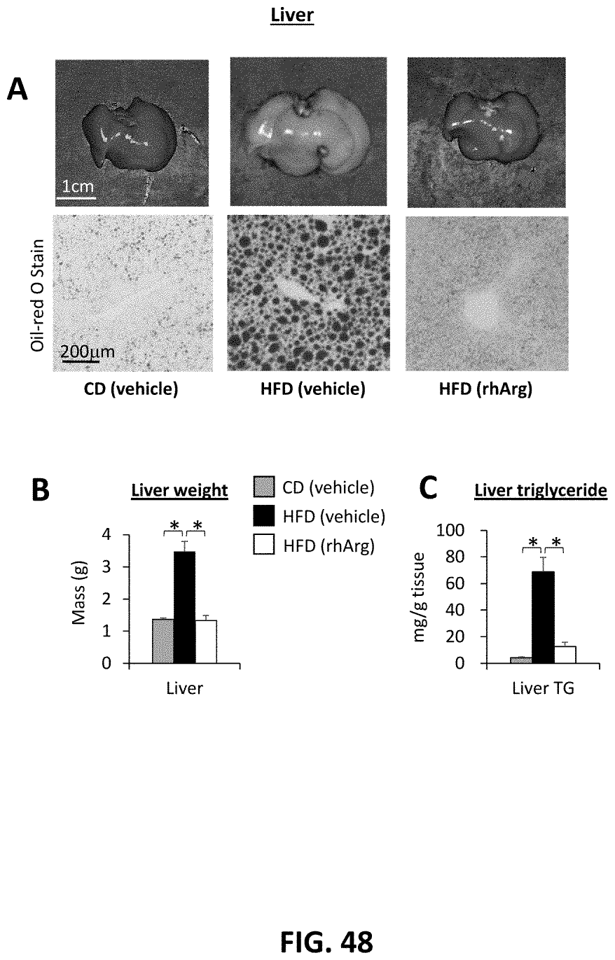

The presence of extensive lipophagy is consistent with reduced lipid content in the liver after N-ABD094-rhArg (SEQ ID NO: 50) treatment. Twelve weeks of HFD feeding induced hepatic steatosis in C57BL/6J male mice (FIG. 48A). After extending the feeding of HFD for 12 weeks, the liver was further enlarged and appeared pale in colour (FIG. 48A). The liver weight was more than double of that of the lean control mice fed a chow diet [CD (vehicle) group] (FIG. 48B). Massive lipid accumulation had extended to the entire liver as indicated by oil red O staining of lipids (FIG. 48A). However, in DIO mice treated with N-ABD094-rhArg (SEQ ID NO: 50) [HFD (rhArg) group], the liver showed a marked diminution in size (FIG. 48A) to such an extent that the weight was similar to the lean control (FIG. 48B). Moreover, the liver restored to a reddish colour, which was well correlated with a dramatic clearance of lipid droplets (FIG. 48A) with the triglyceride concentrations reduced to a level similar to the lean control (FIG. 48C).

Collectively, our findings reveal the marked effect of N-ABD094-rhArg (SEQ ID NO: 50) on reversal of hepatic steatosis via autophagy (lipophagy).

### Example 16

### The N-ABD094-rhArg (SEQ ID NO: 50) Induces Autophagy (Lipophagy) During the Fasting Phase of the 7-Day Intermittent Fasting Cycle to Break Down Lipids in the BAT of C57BL/6J Mice Fed a HFD

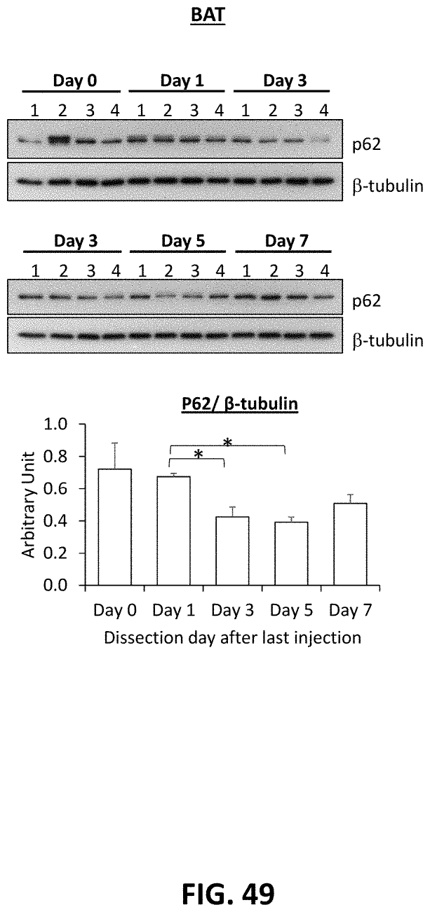

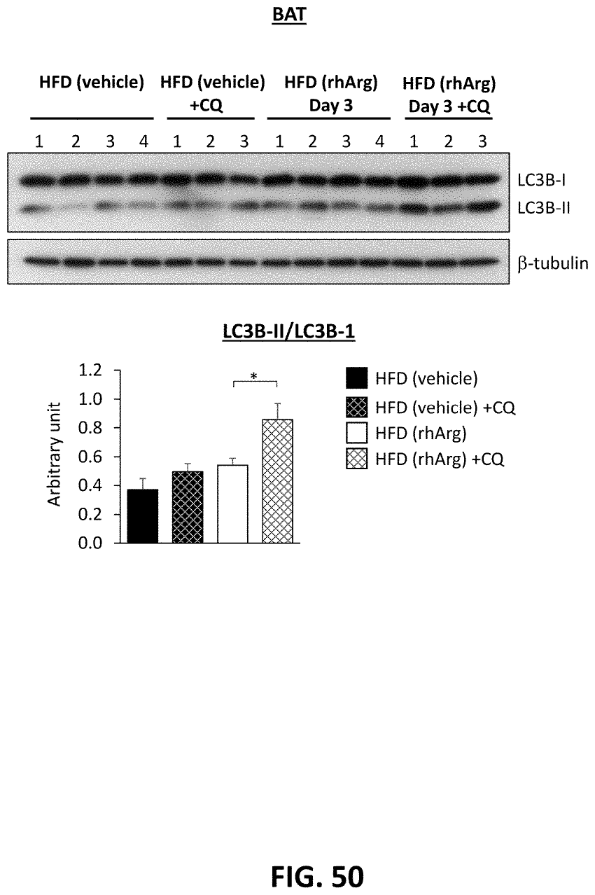

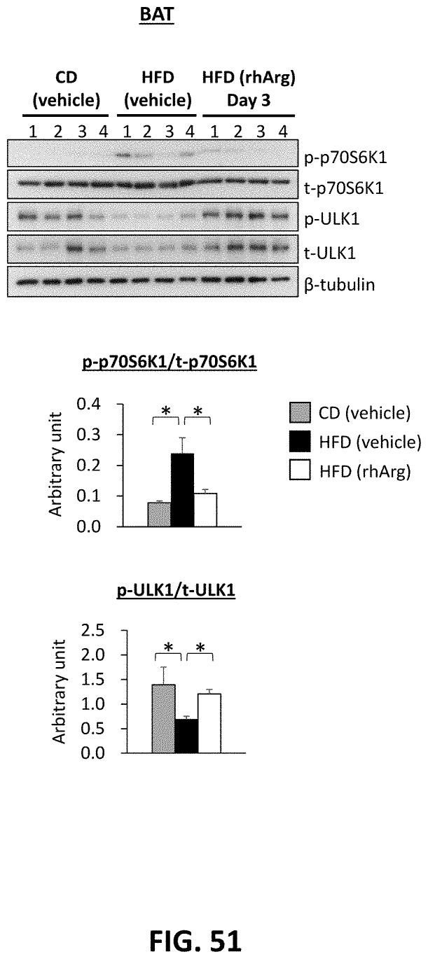

The p62, also known as SQSTMl/sequestome 1, serves as a link between LC3B and ubiquitinated substrates and is efficiently degraded by autophagy. Thus, the level of p62 proteins can be used to monitor autophagic flux. For example, autophagic suppression correlates with an increased p62 level, and similarly, autophagic activation correlates with a decreased p62 level. Western blotting of p62 on brown adipose tissue (BAT) showed that there is a significant decrease in p62 level at Day 3 and Day 5, implying the presence of autophagic flux during the fasting period of the intermittent fasting cycle induced by N-ABD094-rhArg (SEQ ID NO: 50) administration (FIG. 49). Besides, the expression level of autophagy marker LC3B was detected in presence or absence of CQ (FIG. 50). The conversion of LC3B -I to the lower migrating form, LC3B-II, is used as an indicator of cellular autophagy level. The higher the LC3B-II/LC3B-I ratio indicates the higher incidence rate of autophagy in the tissue. In line with the decreased p62 level at Day 3, the result showed that the LC3-II/LC3-I ratio in the BAT sample of the N-ABD094-rhArg (SEQ ID NO: 50)-treated group [HFD (rhArg) group] collected at Day 3 of the 7-day intermittent fasting cycle showed a dramatic increase upon injection of CQ for 5 hrs (FIG. 50). This indicated that N-ABD094-rhArg treatment induced autophagy in the liver at Day 3. In obese mice treated with vehicle [HFD (vehicle) group], there was an absence of increase after CQ administration, suggesting that the tissue was under autophagy arrest.

Nevertheless, the suppression of ribosomal protein S6 kinase beta-1 (p70S6K1, a downstream protein of mammalian target of rapamycin mTOR) and stimulation of Unc-51 like autophagy activating kinase 1 (ULK1, an initiator of autophagy) in BAT of HFD-induced obese male mice administrated with 600 U N-ABD094-rhArg (SEQ ID NO: 50) indicated that the initiation of autophagy may be mediated via the inhibition of mTOR pathway, which is the major sensor of arginine deprivation.

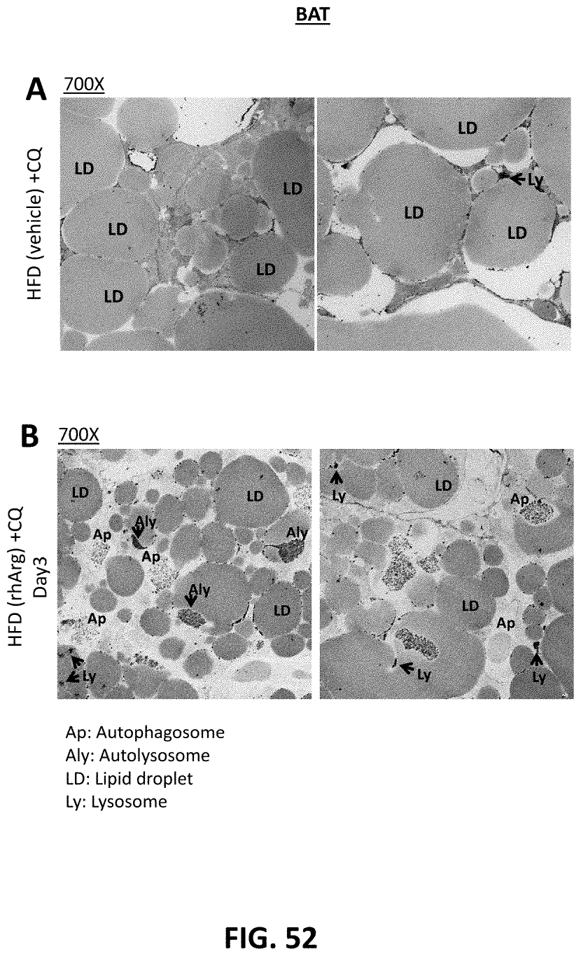

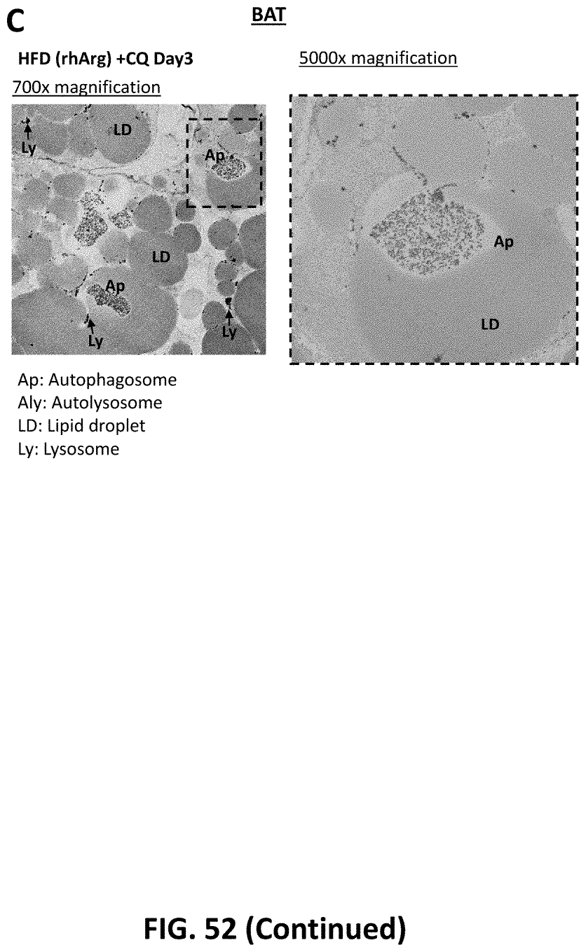

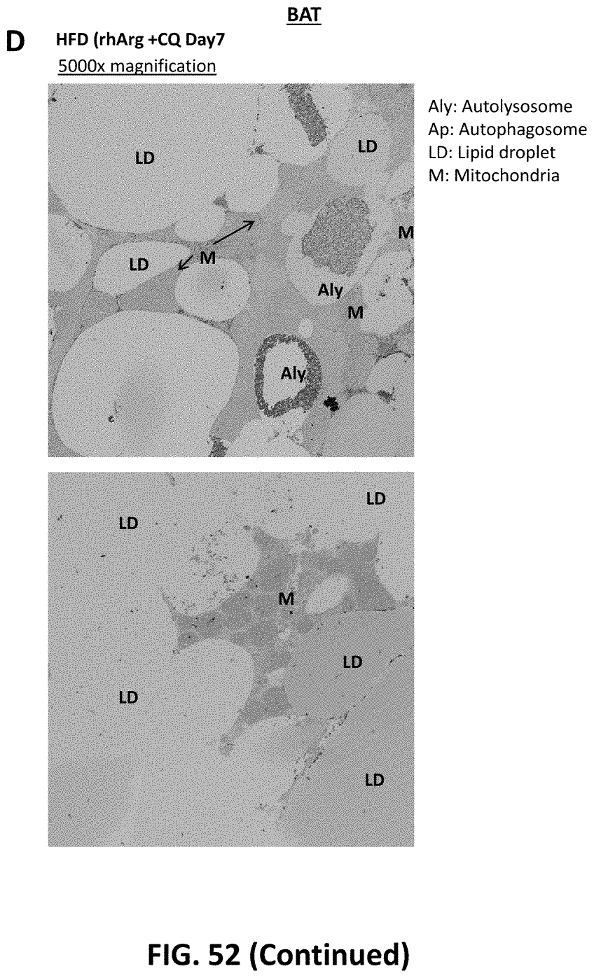

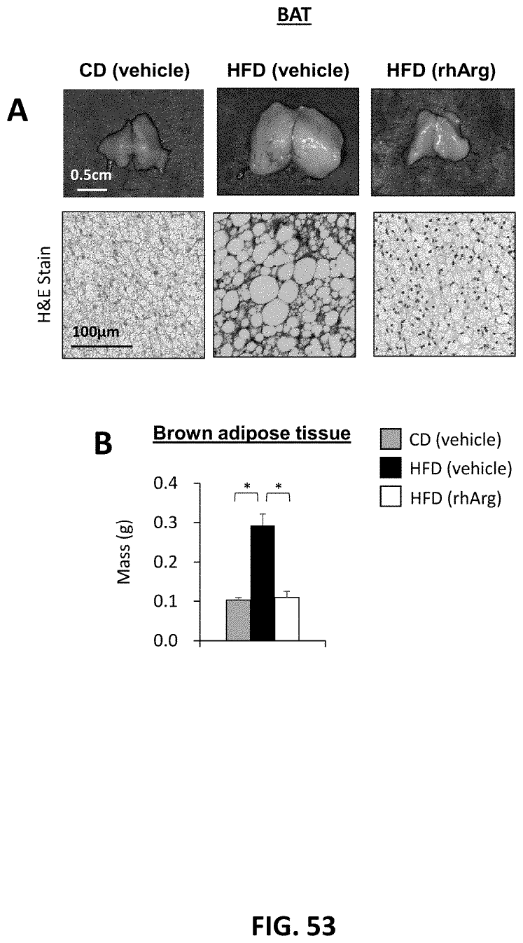

After prolonged feeding on a HFD, there was a further increase in the size (FIG. 53A) and weight of the interscapular BAT (FIG. 53B) in vehicle-treated DIO mice [HFD (vehicle) group] in comparison to the lean control mice [CD (vehicle) group], which was correlated with the presence of massive amount of enlarged white-like unilobular cells (FIG. 53A). As observed under transmission electron microscopy, brown adipocyte in mice fed with HFD [HFD (vehicle) group] had massive enlarged lipid droplets (FIG. 52A). In contrast, brown adipocyte in mice fed with HFD and treated with N-ABD094-rhArg (SEQ ID NO: 50) [HFD (rhArg) group] exhibited active lipophagy at Day 3 of treatment, which is the fasting period of the 7-day intermittent fasting cycle. Many autophagosomes were actively forming and engulfing the lipid droplets (FIG. 52B). The autolysosome was also found to be breaking down small lipid droplets, mixing with plenty of lysosomes in the structure (FIG. 52C). When compared with Day 3, the BAT section from N-ABD094-rhArg (SEQ ID NO: 50) treated group at Day 7 showed that continuous active autophagy (lipophagy) was taking place as there was a considerable amount of autophagosomes and autolysosomes present in the cytoplasm of brown adipocytes (FIG. 52D). Besides, there were extensive areas exhibiting empty cavities with the lipid content therein being completely degraded, implying that autophagy, especially lipophagy, was persistently taking place in the cytoplasm of brown adipocytes. In addition, of note, there was relatively large amount of mitochondria in the cytoplasm of the brown adipocyte in the Day 7 rhArg-treated sample in comparison to that in the Day 3 sample. This likely indicated that mitochondria biogenesis was also actively occurring once lipophagy induced by rhArg dissipated lipid droplets, which reached certain extent.

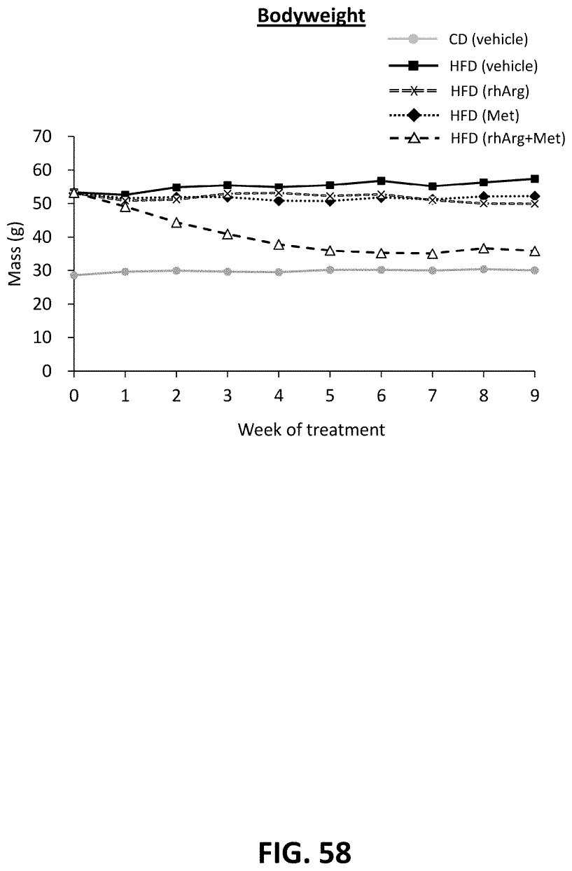

Concomitant with the presence of autophagy, treatment of DIO mice with N-ABD094-rhArg (SEQ ID NO: 50) could dramatically restore the interscapular BAT to a size (FIG. 53A) and weight (FIG. 58B) similar to the lean control mice, with almost complete remission of white-like unilobular cells (FIG. 53B). Collectively, these findings demonstrate that autophagy induced by N-ABD094-rhArg (SEQ ID NO: 50) reverses whitening of the brown fat.

### Example 17

### The N-ABD094-rhArg (SEQ ID NO: 50) Induces Autophagy in Hypothalamic POMC Neurons Leading to Appetite Inhibition

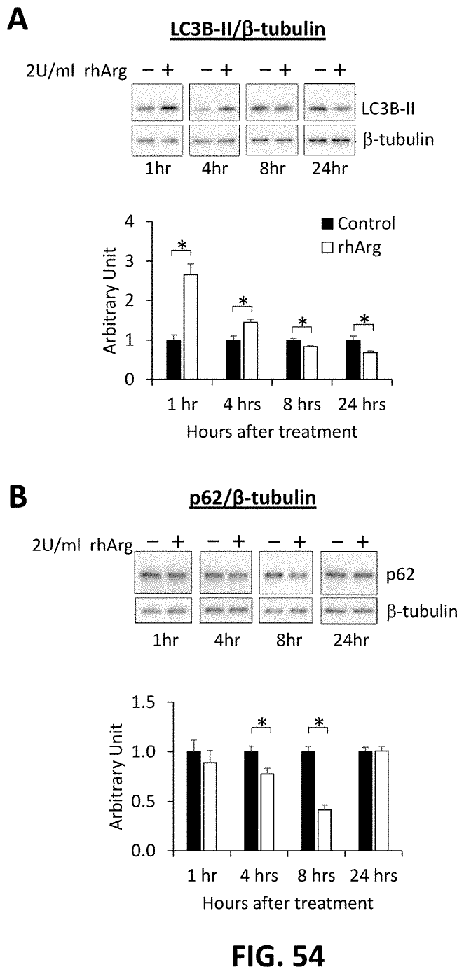

The arcuate nucleus in the hypothalamus has received extensive attention as an integrator and regulator of energy homeostasis and appetite. These neurons can rapidly sense metabolic fluctuations in the blood. Others have recently implicated autophagy in central appetite regulation and leptin sensitivity (Park et al., Nature Communications. 2020; 11: 1914). Knockdown of an essential autophagy gene autophagy-related 7 (Atg7), leads to accumulation of p62 in POMC neurons, resulting in an increase in bodyweight and appetite. Besides, it has been shown that amino acid deprivation/imbalance can trigger a reduction in food intake via eIF2α/ATF4 pathway [Maurin et al., Cell Rep. 2014 Feb. 6(3): 438-444]. In our study, mature hypothalamic neurons (after 14 day in vitro culture) were exposed to vehicle or 2 U/mL N-ABD094-rhArg (SEQ ID NO: 50) for 1 hr, 4 hrs, 8 hrs or 24 hrs. After treatment, proteins were harvested from whole cell lysate of neurons and subjected to western blot analysis for examining the short-term effect of arginine depletion on hypothalamic neurons. Autophagy markers (LC3B and p62) were analyzed. In FIG. 54, it can been seen that after 1 hr and 4 hrs of rhArg treatment, the level of LC3B-II was significantly increased (FIG. 54A), which suggested that there was enhanced formation of autophagosome in primary hypothalamic neurons. Together, the level of p62 was significantly decreased after 4 hrs and 8 hrs of N-ABD094-rhArg (SEQ ID NO: 50) treatment (FIG. 54B), which indicated an increase in autophagosome degradation. These findings supported that N-ABD094-rhArg (SEQ ID NO: 50) induced an autophagic flux in hypothalamic neurons.

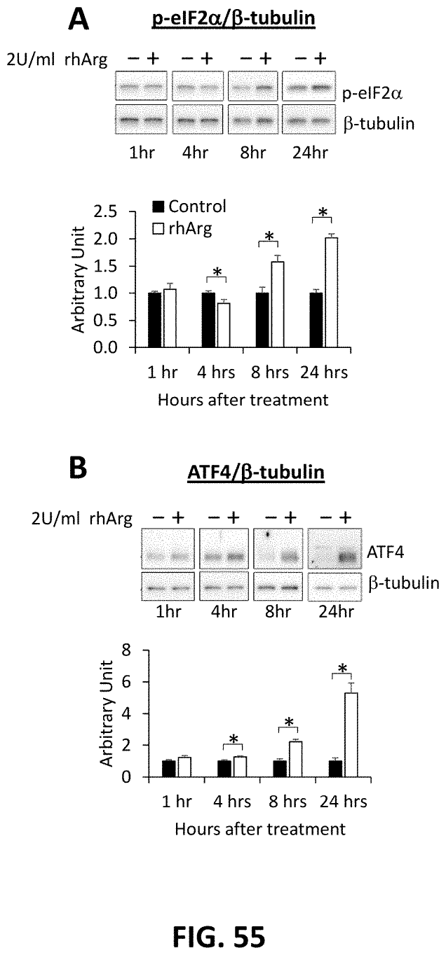

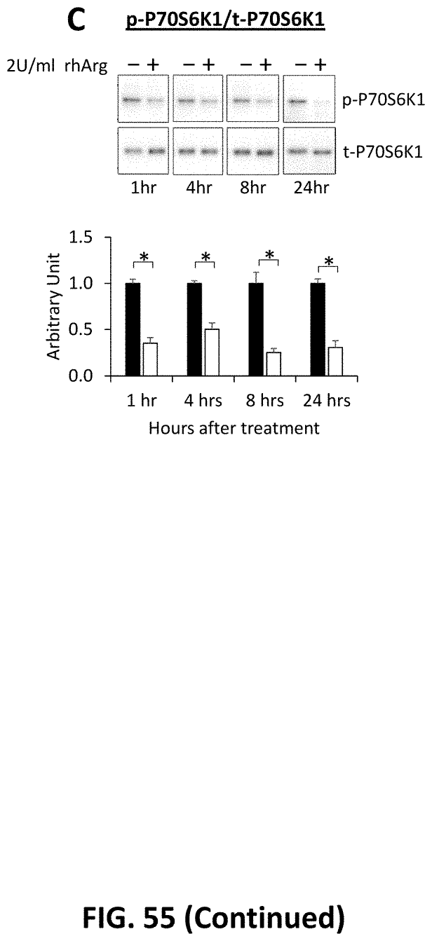

Other than autophagy, a significant increase in the phosphorylation level of eIF2α was observed at 8 hrs or 24 hrs after N-ABD094-rhArg (SEQ ID NO: 50) treatment (FIG. 55A), which suggested that arginine depletion induced activation of eIF2α in hypothalamic neurons. In addition, a dramatic increase in ATF4 level was observed in the rhArg-treated neurons 24 hrs after treatment (FIG. 55B), which is in line with the findings of amino acid deprivation in vivo [Maurin et al., Cell Rep. 2014 Feb. 6(3): 438-444].

Furthermore, treatment with N-ABD094-rhArg (SEQ ID NO: 50) significantly decreased the phosphorylation level of p70S6K1 in neurons after 1 hr, 4 hrs, 8 hrs or 24 hrs of treatment (FIG. 55C). This data suggested that arginine deprivation suppressed activity of p70S6K1 via mTOR pathway in hypothalamic neurons.

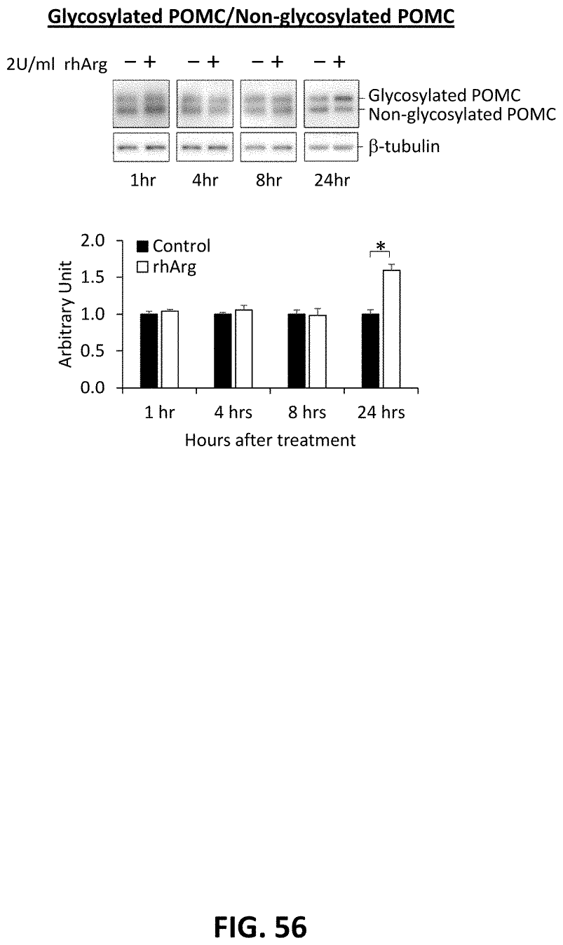

The proopiomelanocortin (POMC) neurons produce the neuro peptide precursor POMC, which is cleaved to form α-melanocyte stimulating hormone (α-MSH), which ultimately reduces food intake. Our data showed that N-ABD094-rhArg (SEQ ID NO: 50) treatment significantly increased the proportion of glycosylated POMC in neurons after 24 hrs of treatment (FIG. 56).

Taken together, the suppression of mTOR pathway is the earliest response towards depletion of arginine, and followed by activation of eIF2α/ATF4 pathway. Finally, upregulation in the proportion of glycosylated POMC was observed after autophagic flux occurred.

Upregulation of autophagy pathway may be neuroprotective, and much effort is being invested in developing drugs that cross the blood-brain barrier and increase neuronal autophagy. One well-recognized way of inducing systemic autophagy is by food restriction, which upregulates autophagy in many organs including the liver and neuronal cells (Alirezaei et al. 2010. Autophagy 6(6):702-710). Thus, sporadic fasting may represent a simple, safe and inexpensive means to promote this potentially therapeutic neuronal response. That means N-ABD094-rhArg (SEQ ID NO: 50) or other forms of arginase (e.g., rhArg-PEG, BCA-PEG or BCA-ABD) or arginine-depleting enzymes (e.g., ADI-PEG, ADI-ABD, ADC-PEG or ADC-ABD), which are shown here to be an efficient intermittent fasting inducer and/o autophagy inducer, can be used to treat or prevent neurodegeneration diseases that are caused by disruption of autophagy.

### Example 18

### Synergistic Effects of Combining N-ABD094-rhArg (SEQ ID NO: 50) and Metformin in Inducing Intermittent Fasting on C57BL/6J Male Mice Fed a HFD

Metformin is the frontline drug for treatment of type2 diabetes.

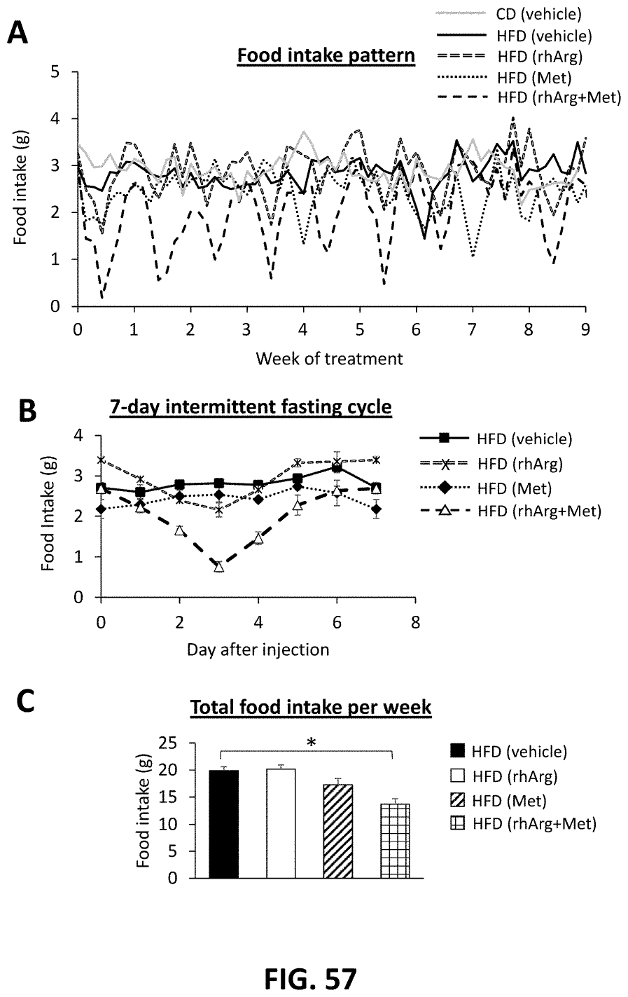

Other than applying alone, we found thatN-ABD094-rhArg (SEQ ID NO: 50) when combined with metformin demonstrate a synergistic interaction in inducing intermittent fasting and reducing adiposity (FIG. 57). Five groups of pre-existing C57BL/6J male mice fed with a HFD for 12 weeks were treated at different conditions. For the combination group, mice were injected once a week with half the dosage of N-ABD094-rhArg (SEQ ID NO: 50, 300 U) together with orally fed of 300 mg/kg metformin once per day [HFD (rhArg+Met)]. The dose of metformin administrated is mimicking the dosage clinically used on humans. For the control group, C57BL/6J obese male mice were either weekly injected with half-dose of N-ABD094-rhArg (SEQ ID NO: 50) together with water daily fed [HFD (rhArg)] or weekly injected with saline together with 300 mg/kg metformin fed daily [HFD (Met)]. Obese mice without N-ABD094-rhArg (SEQ ID NO: 50) and metformin treatment were serve as negative control [HFD (vehicle)] while lean mice with Chow diet [Chow (vehicle)] were serve as a normal control.

In terms of the daily food intake (FIG. 57A), we observed that the food intake of the vehicle treatment or metformin treated group were stable, and they consumed about 2.5 to 3 grams of food pellet daily. When the mice were treated with 300 U N-ABD094-rhArg (SEQ ID NO: 50) once weekly alone, even though it was a half dosage, we could observe a weak intermittent fasting cycle (FIG. 57B). Similarly, from Day 1 to Day 3, the food intake decreased continuously. The food consumption reached the least on Day 3 but the magnitude of appetite suppression is not as strong as full dose 600 U/week. After that the mice resumed food intake from Day 4 to Day 7. Surprisingly, when the mice received drug treatment with both 300 U N-ABD094-rhArg (SEQ ID NO: 50) weekly and 300 mg/kg metformin daily, we could observe a significant intermittent fasting cycle that is as strong as the full dose administration of 600 U N-ABD094-rhArg (SEQ ID NO: 50) that describe previously (FIG. 1) (FIG. 57C) In parallel with the food consuming, we could observe that the average body weight of the chow group was very stable and they kept at a level of about 30 grams during the treatment period, while the HFD group mice continued to increase slightly from around 51 g at the beginning to about 58 g with 11 weeks continuous HFD feeding (FIG. 58). Mice upon single drug treatment with 300 U N-ABD094-rhArg (SEQ ID NO: 50) alone weekly or feeding of 300 mg/kg metformin alone daily for about 11 weeks also demonstrate a stable body weight without further increase in body mass even fed with HFD (FIG. 58). In contrast, mice received combination therapy with both N-ABD094-rhArg (SEQ ID NO: 50) and metformin for 11 weeks, we could observe that the average body weight of the mice continued to drop from 50 grams to 35 grams within 4 weeks, and then maintain a steady state until the end of 11 weeks of treatment (FIG. 58). The combination uses of N-ABD094-rhArg (SEQ ID NO: 50) and metformin had a superior synergistic effect on weight loss in the mice.

Together, these findings support that treatment of ABD-rhArg fusion protein or other forms of arginase (e.g., rhArg-PEG, BCA-PEG or BCA-ABD) or arginine-depleting enzymes (e.g., ADI-PEG, ADI-ABD, ADC-PEG or ADC-ABD) can induce a synergistic effect on intermittent fasting when combined with metformin.

### Example 19

### Synergistic Effects of Combining N-ABD094-rhArg (SEQ ID NO: 50) and Metformin in Reducing Organ Fat Mass, Reversing Insulin Resistance and Glucose Intolerance on C57BL/6J Male Mice Fed a HFD

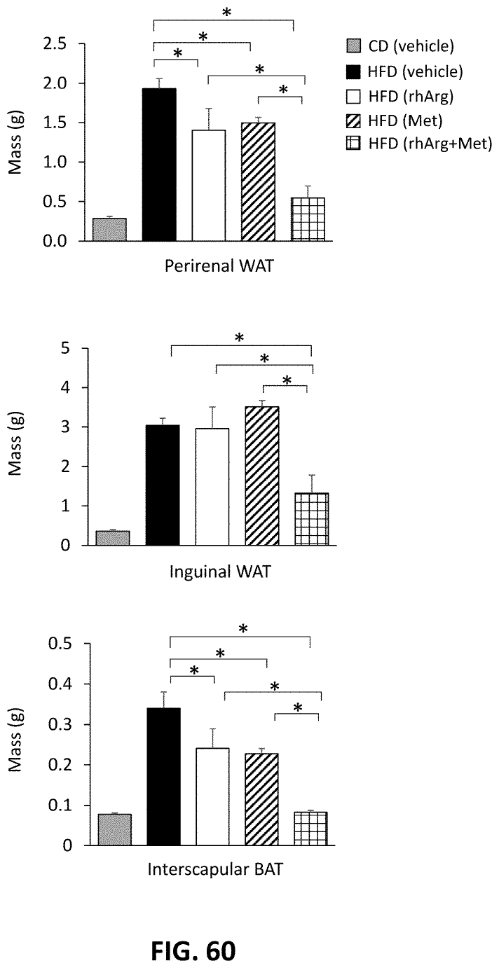

Concomitant with the reduced body weight, there was dramatic reduction of visceral (perirenal) and subcutaneous (inguinal) fat mass (FIG. 60) in combination treatment of both N-ABD094-rhArg (SEQ ID NO: 50) and metformin. Use of N-ABD094-rhArg (SEQ ID NO:50) or metformin individually did not reduce body fat which is concomitant with the unchanged body weight. As previously described, there was a prominent increase in the weight of liver, kidney, pancreas and BAT in DIO mice treated with vehicle. Our results demonstrated that N-ABD-rhArg (SEQ ID NO: 50) or metformin alone cannot reduce the mass of most organs including liver, kidney and BAT (except pancreas, treatment of half dose N-ABD-rhArg alone can also reverse the fatty pancreas). However, combination of both N-ABD-rhArg (SEQ ID NO: 50) and metformin can effectively reverse mass/steatosis in these organs (FIG. 44B).

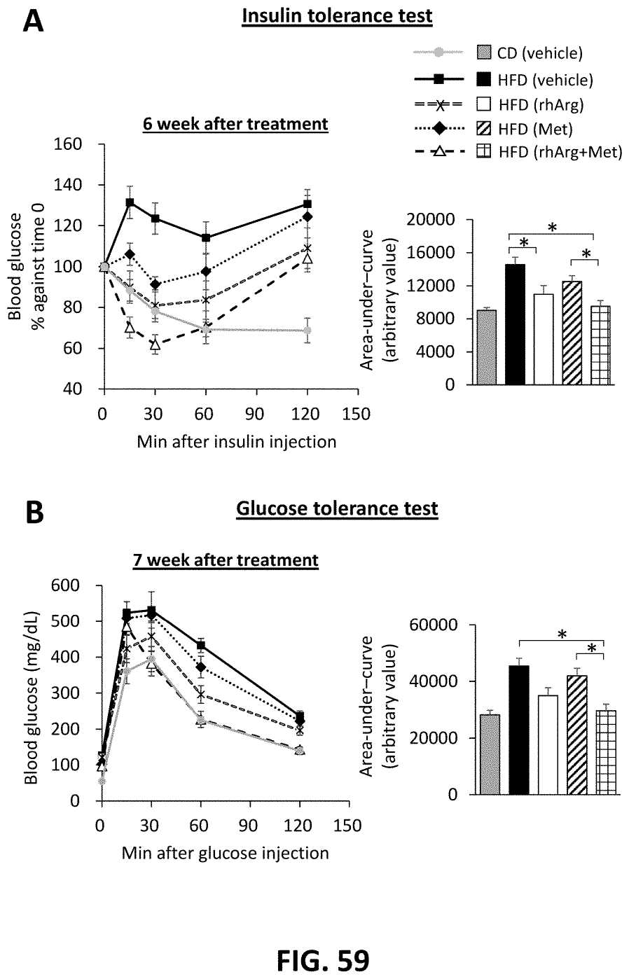

In terms of reversing insulin resistance on pre-existing obese mice, insulin tolerance test (ITT) was employed (FIG. 59). As mentioned previously, mice fed with HFD exhibited impaired insulin response. Results showed that after 6 weeks of treatment, 300 mg/kg metformin treatment alone cannot relieve insulin resistant; treatment with 300 U/week N-ABD-rhArg (SEQ ID NO: 50) alone has a marginal effect on improving insulin sensitivity but not reach statistical significant. While in contrast, a significant enhancement of insulin sensitivity is detected by combination therapy of both N-ABD-rhArg (SEQ ID NO: 50) and metformin reaching a sensitivity level similar to normal lean control mice.

Mice fed with HFD also exhibited impaired glucose tolerance. Results showed that after 7 weeks of treatment, again 300 mg/kg metformin treatment alone cannot relieve glucose intolerance; treatment with 300 U/week N-ABD-rhArg (SEQ ID NO: 50) alone has a significant improvement on glucose tolerance (FIG. 60B). Similarly, a significant enhancement of glucose tolerance is detected by combination therapy of both N-ABD-rhArg (SEQ ID NO: 50) and metformin reaching a level similar to normal lean control mice. In summary, the combination use of the arginase and metformin on the mice could significantly enhance the insulin sensitivity and improve the glucose tolerance, which ultimately could reverse type 2 diabetes in the obese mice.

Together, these findings support that treatment of ABD-rhArg fusion protein or other forms of arginase (e.g., rhArg-PEG, BCA-PEG or BCA-ABD) or arginine-depleting enzymes (e.g., ADI-PEG, ADI-ABD, ADC-PEG or ADC-ABD) can induce a synergistic effect on reducing fat accumulation in organs and improving insulin sensitivity and glucose intolerance when combined with metformin.

### Example 20

### Synergistic Effects of Combining N-ABD094-rhArg (SEQ ID NO: 50) and Metformin in Inducing Autophagy in Liver of C57BL/6J Male Mice Fed a HFD

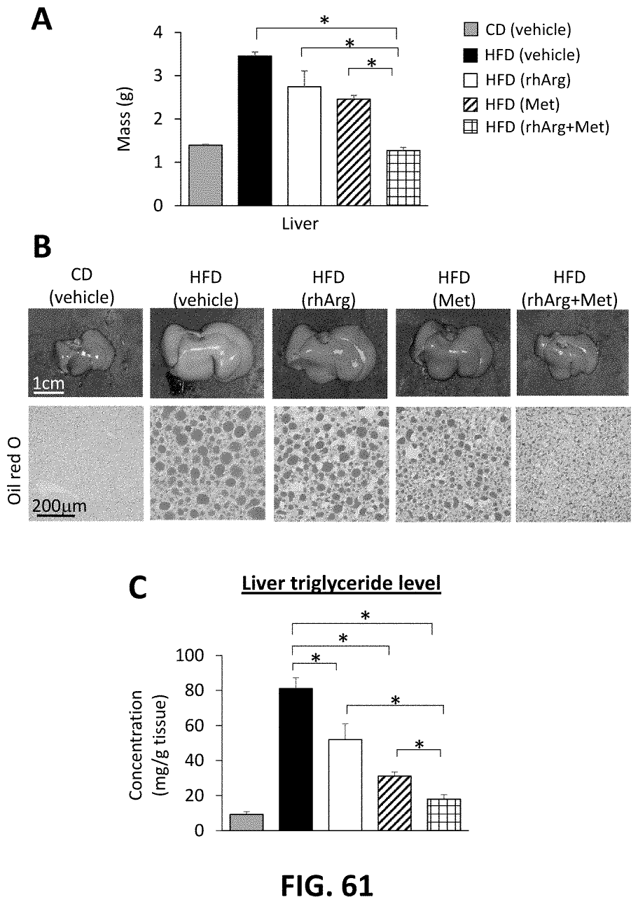

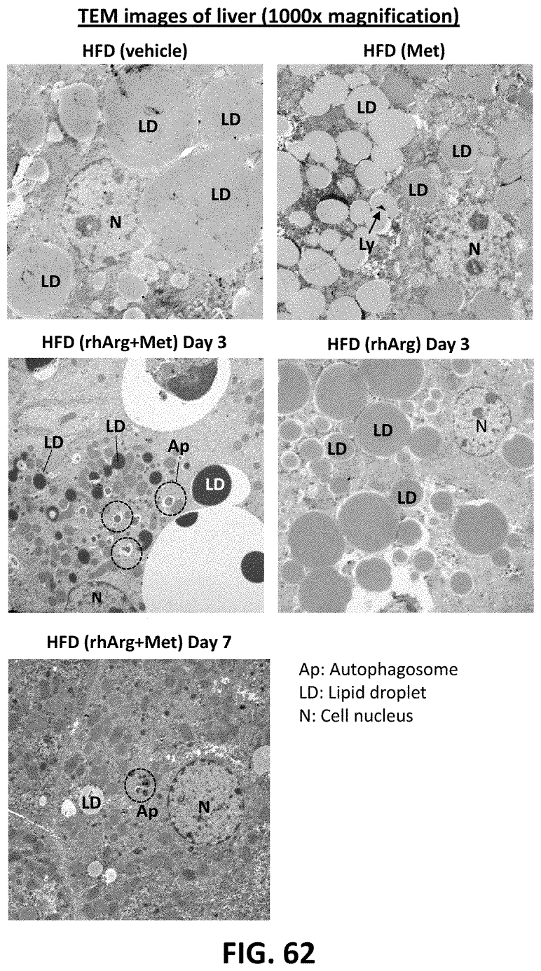

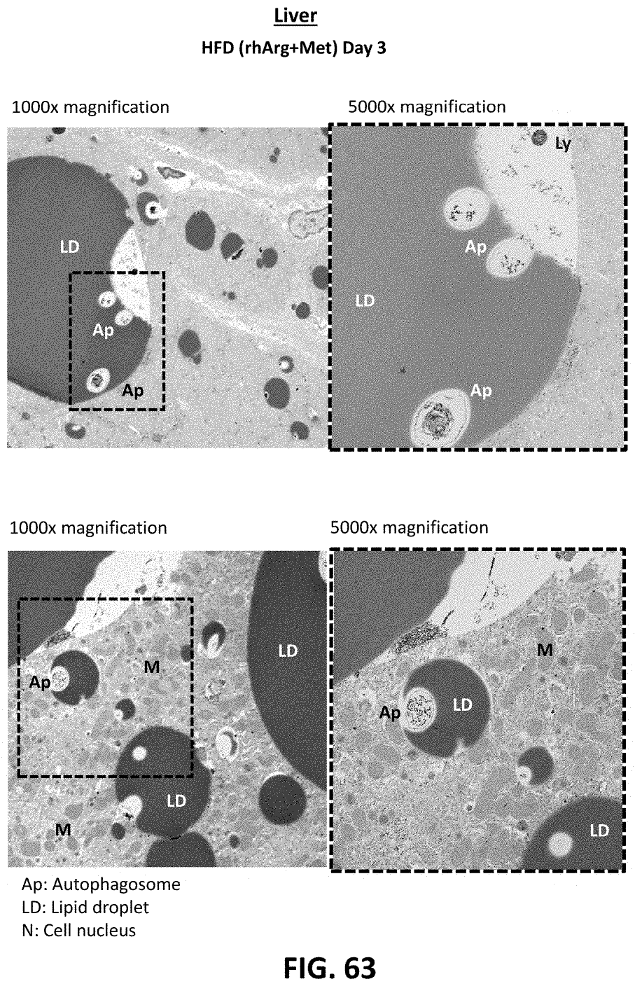

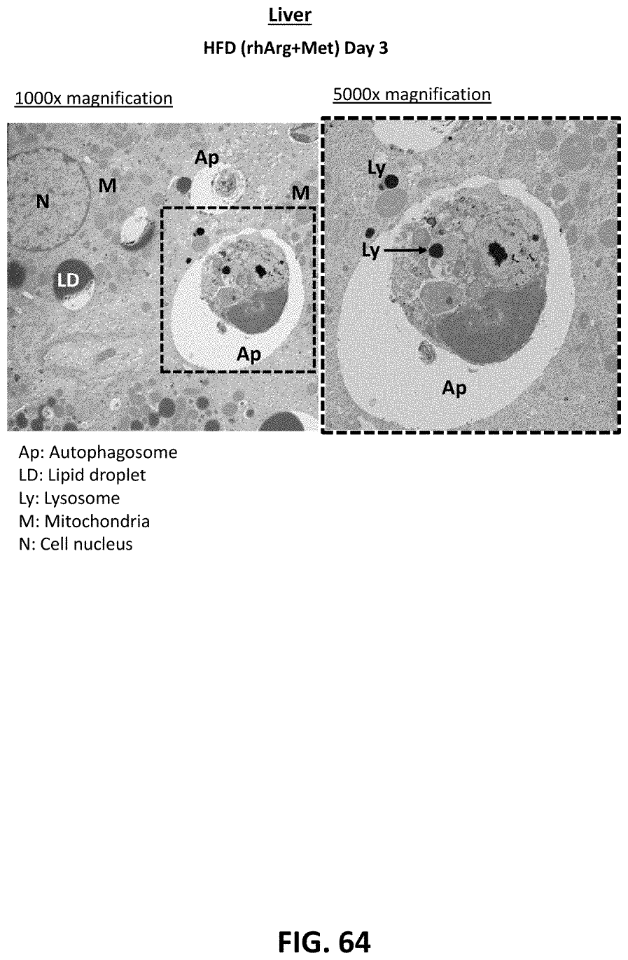

The presence of autophagosome in liver after treatment for 3 weeks were examined by TEM (FIGS. 62, 63 and 64). As mentioned, after prolonged feeding on a HFD, there was an increase in the size (FIG. 61A) of liver which correlated with the accumulation of large lipid droplet in vehicle-treated DIO. As observed by TEM, liver in mice fed with HFD presented massive enlarged lipid droplets (FIG. 61B). Treatment with 300 mg/kg metformin or 300 U/week N-ABD-rhArg (SEQ ID NO: 50) alone did show a significant decrease in lipid droplet size, but no autophagosome is observed in both treatment condition (FIG. 62). In contrast, liver in mice fed with HFD and treated with both N-ABD094-rhArg (SEQ ID NO: 50) and metformin simultaneously can obviously clear most of the lipid droplet in hepatocyte at Day 3 of treatment which is the fasting period of intermittent fasting cycle (FIG. 63). Besides, autophagy is triggered in cell as a massive amount of active autophagosome was observed (FIG. 64). Under 5000× magnification, we found lipophagy was actively taking place via generating autophagosomes that sequestered portions of large lipid droplets to form the double-membrane vesicles, breaking down the droplet into a smaller and more digestible size (FIG. 64). Nevertheless, macroautophagy was also observed characterized by a large autophagosome containing a variety of cytoplasmic components fusing with lysosomes to further form an autolysosome (FIG. 64).

General H&E staining of liver (FIG. 61) at 11 weeks after treatment showed a result in parallel with the observation in TEM at third week of treatment. Combine treatment of N-ABD094-rhArg (SEQ ID NO: 50) and metformin presents an extreme positive effect that reverse non-alcoholic fatty liver. Single treatment of low dose N-ABD094-rhArg (SEQ ID NO: 50) or normal dose Metformin also have slight effect of diminishing lipid in liver in absence of autophagy, suggesting that lipophagy is not the only mechanism in reducing lipid content in liver. Taken together, the induction of autophagy by combination treatment of N-ABD094-rhArg (SEQ ID NO: 50) and metformin can greatly help the removal of lipid droplet in hepatocytes and reversing fatty liver.

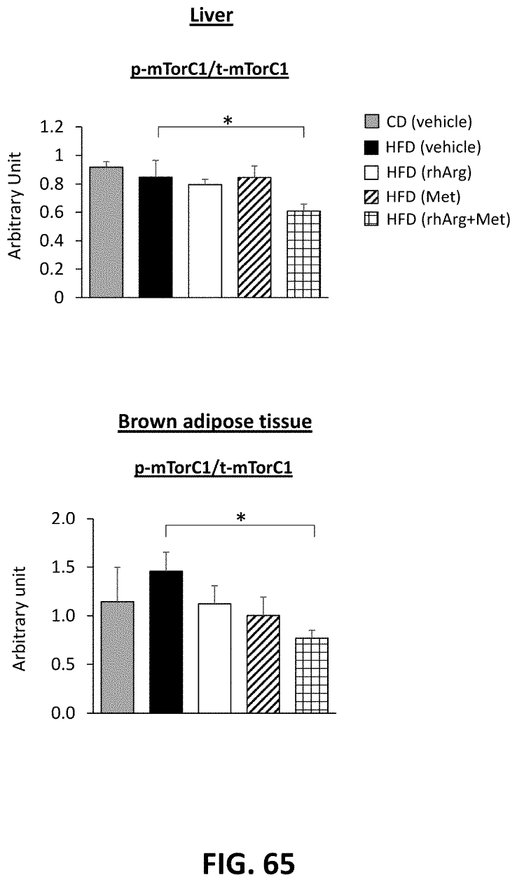

The induction of autophagy is believed to be correlated with the suppression of mTOR phosphorylation (FIG. 65A), which is the master arginine sensor and autophagy suppressor. Inhibition of mTOR pathway has been reported to enhance cellular autophagy. Similar suppression of mTOR and downstream S6K1 was also observed in BAT (FIG. 65B), indicated that autophagy may also be happening in BAT.

### Example 21

### Synergistic Effects of Combining N-ABD094-rhArg (SEQ ID NO: 50) and All-Trans Retinoic Acid (RA) in Inducing Intermittent Fasting on C57BL/6J Male Mice Fed a HFD

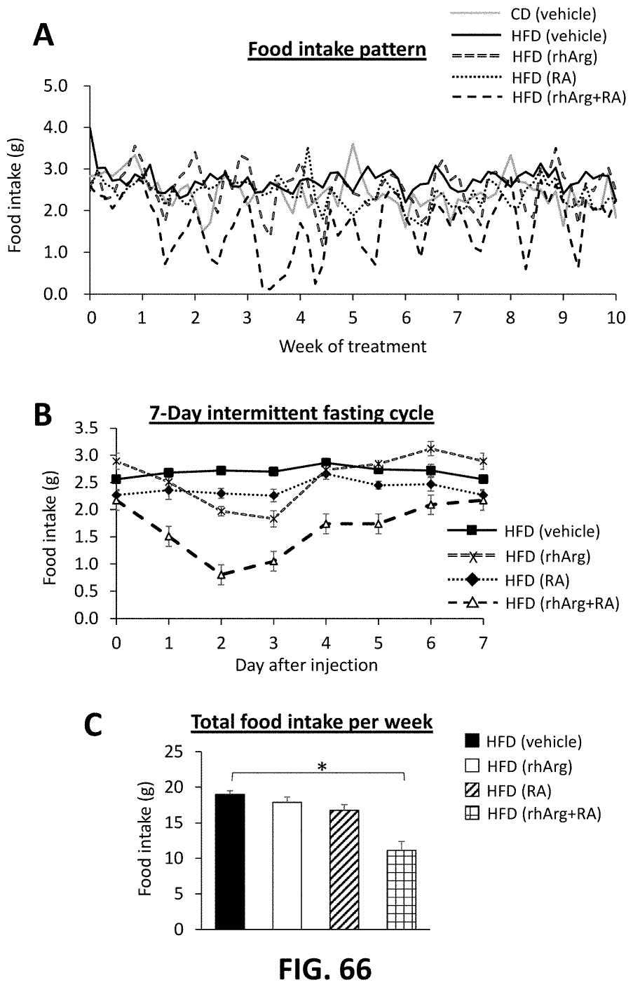

Other than metformin, we found thatN-ABD094-rhArg (SEQ ID NO: 50) when combining with retinoic acid (RA) can also demonstrate a synergistic interaction in inducing intermittent fasting and reducing adiposity (FIG. 66). Five groups of pre-existing C57BL/6J male mice fed with a HFD for 12 weeks were treated at different conditions. For the combination group, mice were weekly injected with half-dose of N-ABD094-rhArg (SEQ ID NO: 50, 200 U) together with orally fed with 0.33 mg RA suspended in peanut oil once per day [HFD (rhArg+RA)]. For the control group, C57BL/6J obese male mice were either weekly injected with half-dose of N-ABD094-rhArg (SEQ ID NO: 50) together with peanut oil feeding daily [HFD (rhArg)] or weekly injected with saline together with 0.33 mg RA fed daily [HFD (RA)]. Obese mice without N-ABD094-rhArg (SEQ ID NO: 50) and RA treatment were serve as negative control [HFD (vehicle)] while lean mice with Chow diet [Chow (vehicle)] were serve as a normal control.

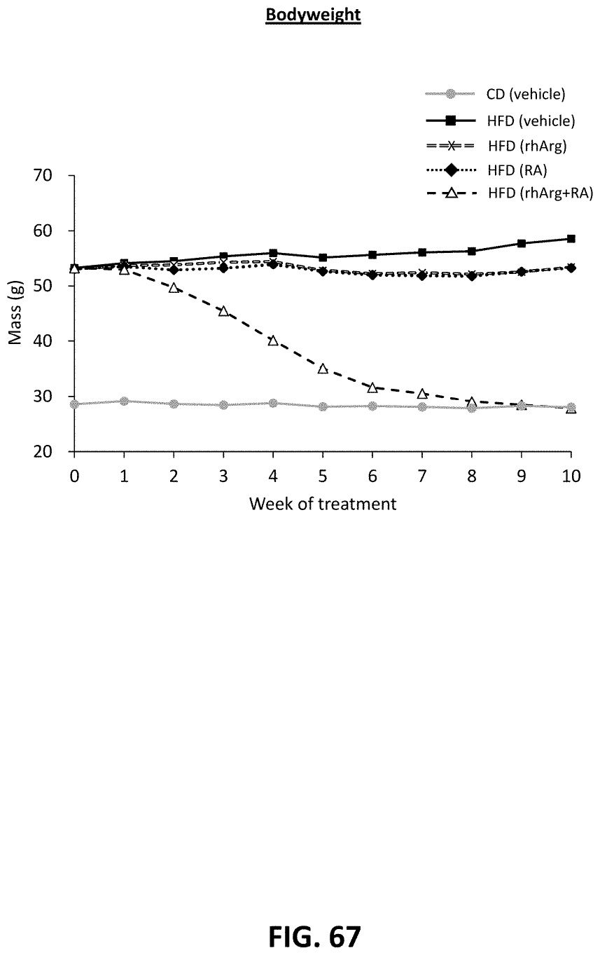

In terms of the daily food intake (FIG. 66B) similar to the combination experiment with metformin, the food intake of the vehicle treatment or RA treated group were stable, and they consumed about 2.5 to 3 grams of food pellet daily. When the mice were treated with 300 U N-ABD094-rhArg (SEQ ID NO: 50) alone weekly, even though it was a half dose, we could observe a weak 7 days intermittent fasting cycle, but the magnitude of appetite suppression is not as strong as full dose 600 U/week. Interestingly, when the mice received drug treatment with both 300 U N-ABD094-rhArg (SEQ ID NO: 50) weekly and 0.33 mg RA daily, we could observe a very clear intermittent fasting cycle that is as strong as the full dose administration of 600 U N-ABD094-rhArg (SEQ ID NO: 50) that describe previously (FIG. 66B). In parallel with the food consuming, we could observe that the average body weight of the chow group was very stable and they kept at a level of about 30 grams during the treatment period, while the HFD group mice continued to increase slightly from around 52 g at the beginning to about 60 g with 11 weeks continuous HFD feeding (FIG. 66B). Mice upon single drug treatment with 300 U N-ABD094-rhArg (SEQ ID NO: 50) alone weekly or feeding of 0.33 mg RA alone daily for about 11 weeks also demonstrate a stable body weight without further increase even fed with HFD (FIG. 67). In contrast, mice received combination therapy with both N-ABD094-rhArg (SEQ ID NO: 50) and RA for 11 weeks, we could observe that the average body weight of the mice continued to drop from 52 g to 30 g within 6 weeks, and then maintain a steady state until the end of 11 weeks' treatment (FIG. 67). The combination uses of N-ABD094-rhArg (SEQ ID NO: 50) and RA had a superior synergistic effect on weight loss in the mice.

Together, these findings support that treatment of ABD-rhArg fusion protein or other forms of arginase (e.g., rhArg-PEG, BCA-PEG or BCA-ABD) or arginine-depleting enzymes (e.g., ADI-PEG, ADI-ABD, ADC-PEG or ADC-ABD) can induce a synergistic effect on intermittent fasting when combined with RA.

### Example 22

### Synergistic Effects of Combining N-ABD094-rhArg (SEQ ID NO: 50) and All-Trans Retinoic Acid (RA) in Reducing Organ Fat Mass, Reversing Insulin Resistance and Glucose Intolerance on C57BL/6J Male Mice Fed a HFD

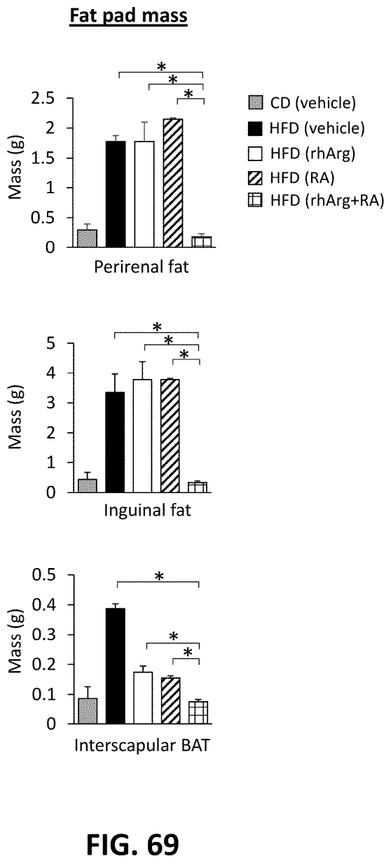

Concomitant with the reduced body weight, there was dramatic reduction of visceral (perigonadal, perirenal and mesenteric fad pad) and subcutaneous (inguinal fat pad) fat mass in combination treatment of both N-ABD094-rhArg (SEQ ID NO: 50) and RA (FIG. 69). Use of N-ABD094-rhArg (SEQ ID NO: 50) or RA individually did not reduce body fat which is concomitant with the unchanged body weight. As previously described, there was a prominent increase in the weight of liver, kidney, pancreas and BAT in DIO mice treated with vehicle. Our results demonstrated that N-ABD-rhArg (SEQ ID NO: 50) or RA alone can already reduce the mass of most organs including liver, pancreas and BAT but the magnitude of reduction is not as strong as combination of both N-ABD-rhArg (SEQ ID NO: 50) and RA (FIG. 53B).

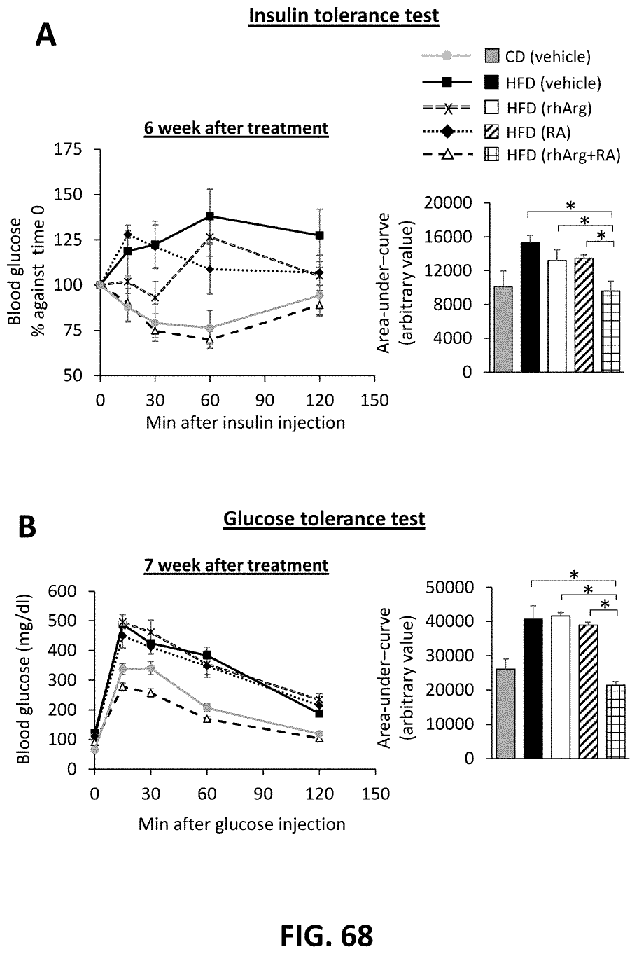

In terms of reversing insulin resistance on pre-existing obese mice, insulin tolerance test (ITT) was employed (FIG. 68A). As mentioned previously, mice fed with HFD exhibited impaired insulin response. Results showed that after 6 weeks of treatment, 300 U/week N-ABD-rhArg (SEQ ID NO: 50) or 0.33 mg/day RA treatment alone has a marginal effect on improving insulin sensitivity but not reach statistical significant. While in contrast, a significant enhancement of insulin sensitivity is detected by combination therapy of both N-ABD-rhArg (SEQ ID NO: 50) and RA reaching a sensitivity level similar to normal lean control mice.

Mice fed with HFD also exhibited impaired glucose tolerance (FIG. 68B). Results showed that after 7 weeks of treatment, again 0.33 mg/day RA or 300 U/week N-ABD-rhArg (SEQ ID NO: 50) alone treatment alone cannot relieve glucose intolerance. On the other hand, a significant enhancement of glucose tolerance is detected by combination therapy of both N-ABD-rhArg (SEQ ID NO: 50) and RA reaching a level similar to normal lean control mice. In summary, the combination use of the arginase and RA on the mice could significantly enhance the insulin sensitivity and improve the glucose tolerance, which ultimately could reverse type 2 diabetes in the obese mice.

### Example 23

### Synergistic Effects of Combining N-ABD094-rhArg (SEQ ID NO: 50) and All-Trans RA (RA) in Inducing Autophagy in Liver of C57BL/6J Male Mice Fed a HFD

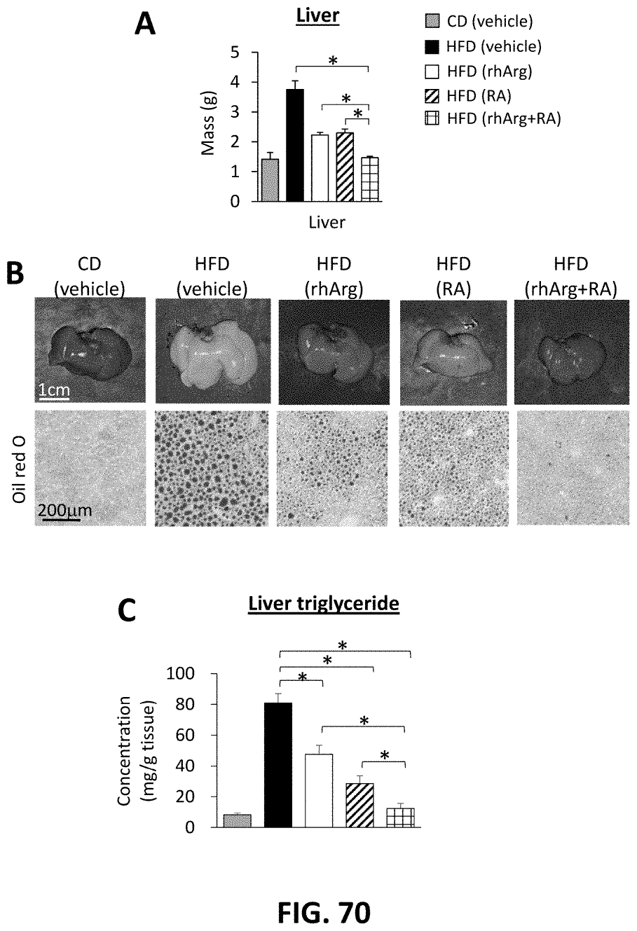

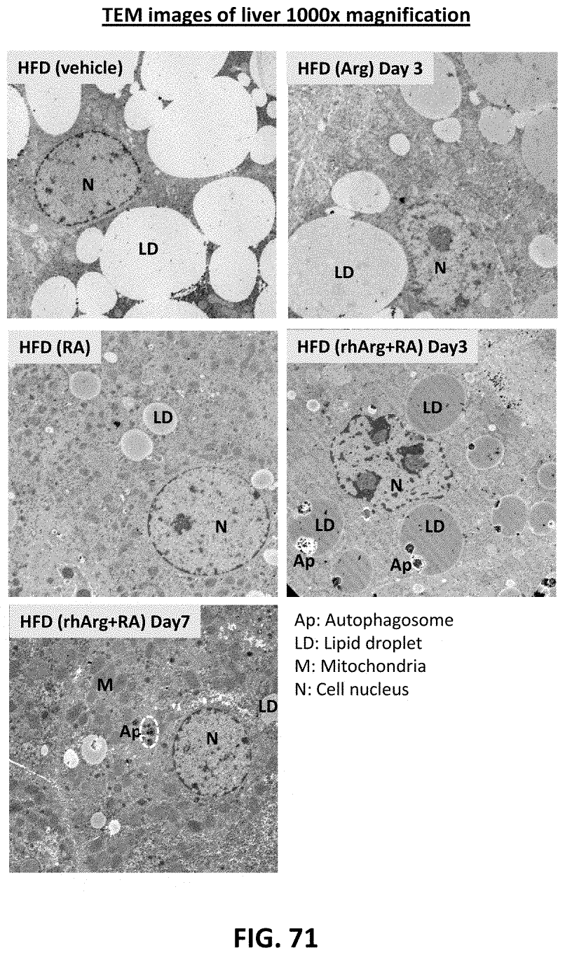

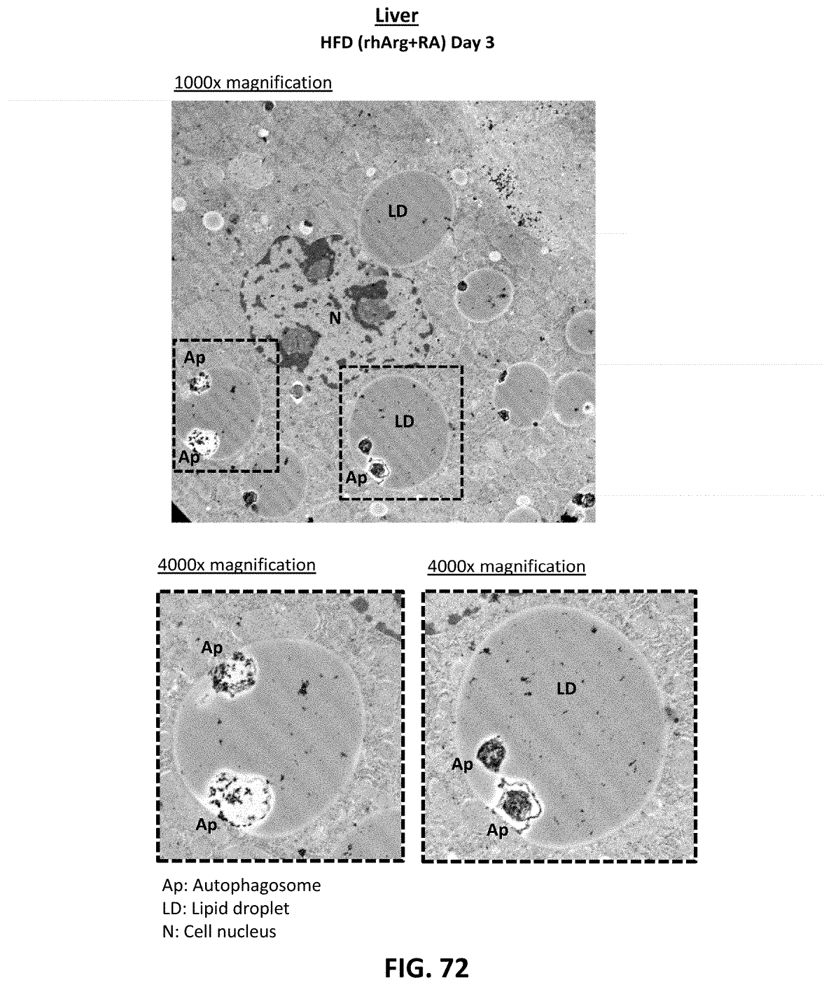

The presence of autophagosome in liver after treatment for 3 weeks were examined by transmission electron microscopy (FIGS. 71 and 72). As mentioned, after prolonged feeding on a HFD, there was an increase in the size (FIG. 70) of liver which correlated with the accumulation of large lipid droplet in vehicle-treated DIO. As observed by TEM, liver in mice fed with HFD presented massive enlarged lipid droplets (FIG. 71). Treatment with 0.33 mg RA daily or 300 U/week N-ABD-rhArg (SEQ ID NO: 50) alone already demonstrated a significant decrease in lipid droplet size, with the effect of RA alone more significant than N-ABD-rhArg alone. However, no autophagosome is observed in both treatment condition (FIG. 71). In contrast, liver in mice fed with HFD and treated with both N-ABD094-rhArg (SEQ ID NO: 50) and RA simultaneously can obviously clear most of the lipid droplet in hepatocyte at Day 3 of treatment which is the fasting period of intermittent fasting cycle (FIG. 72), the result presents an extreme positive effect that reverse non-alcoholic fatty liver. Besides, autophagy is triggered in cell as a massive amount of active autophagosome was observed (FIG. 71). Under 4000× magnification, we found active lipophagy were taking place via generating autophagosomes that sequestered portions of large lipid droplets to form the double-membrane vesicles, breaking down the droplet into a smaller and more digestible size (FIG. 71).

H&E staining of liver (FIG. 70) at 11 weeks after treatment showed combine treatment of N-ABD094-rhArg (SEQ ID NO: 50) and RA. Single treatment of low dose N-ABD094-rhArg (SEQ ID NO: 50) or normal dose RA also have significant effect on diminishing lipid in liver. On the other hand, combination treatment of N-ABD094-rhArg (SEQ ID NO: 50) and RA can completely remove the lipid droplet in hepatocytes and reversing fatty liver.

Together, these findings support that treatment of ABD-rhArg fusion protein or other forms of arginase (e.g., rhArg-PEG, BCA-PEG or BCA-ABD) or arginine-depleting enzymes (e.g., ADI-PEG, ADI-ABD, ADC-PEG or ADC-ABD) can induce a synergistic effect on reducing fat accumulation in organs and improving insulin sensitivity and glucose intolerance when combined with RA.

We claim:

1. A method of inducing intermittent fasting, modulating autophagy, or inducing intermittent fasting and modulating autophagy in a subject in need thereof comprising the step of administering a therapeutically effective amount of an arginine depleting agent to the subject.

2. The method of claim 1, wherein inducing intermittent fasting, modulating autophagy, or inducing intermittent fasting and modulating autophagy in the subject results in treatment of at least one autophagy related or intermittent fasting related disease or health condition selected from the group consisting of increasing the longevity of the subject, a symptom of aging or preventing an age related disease, and promoting cellular regeneration.

3. The method of claim 1, wherein the arginine concentration in the subject's serum is maintained below 50 μM, below 25 μM, below 20 μM, below 10 μM, or below 5 μM.

4. The method of claim 1, wherein the arginine depleting agent is an arginase protein, an arginine deiminase protein, or an arginine decarboxylase protein.

5. The method of claim 4, wherein the arginase protein, arginine deiminase protein, or arginine decarboxylase protein further comprises one or more polyethylene glycol (PEG) groups.

6. The method of claim 5, wherein the arginase protein comprises a polypeptide having SEQ ID NO: 101, SEQ ID NO: 102, SEQ ID NO: 103, or SEQ ID NO: 104.

7. The method of claim 4, wherein the arginase protein, arginine deiminase protein, or arginine decarboxylase protein further comprises an albumin binding domain or human serum albumin, or a human IgG Fc domain.

8. The method of claim 7, wherein the arginine depleting agent is a fusion protein comprising an ABD polypeptide and an arginase polypeptide; an ABD polypeptide and an arginine deiminase polypeptide; or an ABD polypeptide and an arginine decarboxylase polypeptide.

9. The method of claim 1, wherein the arginine depleting agent comprises a polypeptide having at least 98% sequence homology with SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 75, SEQ ID NO: 107, or SEQ ID NO: 76.

10. The method of claim 1, wherein the arginine depleting agent is co-administered with a therapeutically effective amount of an autophagy inducing agent.

11. The method of claim 10, wherein the autophagy inducing agent is selected from the group consisting of a retinoid derivative, an (−)-epigallocatechin-3-gallate (EGCG) derivative, a green tea catechin, and a rapamycin derivative.

12. The method of claim 10, wherein the autophagy inducing agent is selected from the group consisting of carbamazepine, clonidin, lithium, metformin, rapamycin (and rapalogs), rilmenidine, sodium valproate, verapamil, trifluoperazine, statins, tyrosine kinase inhibitors, BH3 mimetics, caffeine, omega-3 polyunsaturated fatty acids, resveratrol, spermidine, vitamin D, trehalose, polyphenol(−)-epigallocatechin-3-gallate and combinations thereof.

13. The method of claim 1, wherein the arginine depleting agent is co-administered with a therapeutically effective amount of a glucose lowering agent.

14. The method of claim 13, wherein the glucose lowering agent is an alpha-glucosidase inhibitor, a biguanide, bile acid sequestrant, a dopamine-2 agonist, a dipeptidyl peptidase 4 (DPP-4) inhibitor, a meglitinide, a sodium-glucose transport protein 2 (SGLT2) inhibitor, a sulfonylurea, a thiazolidinedione, or a combination thereof.

15. The method of claim 14, wherein the biguanide is metformin; the alpha-glucosidase inhibitor is acarbose or miglitol; the bile acid sequestrant is colesevelam; the dopamine-2 agonist is bromocriptine; the DPP-4 inhibitor is alogliptin, linagliptin, saxagliptin, or sitagliptin; the meglitinide is nateglinide or repaglinide; the SGLT2 inhibitor is canagliflozin, dapagliflozin, or empagliflozin; the sulfonylureas ischlorpropamide, glimepiride, glipizide, or glyburide; and the thiazolidinedione is rosiglitazone or pioglitazone.

16. The method of claim 1, wherein the arginine depleting agent is co-administered with a therapeutically effective amount of a retinoid derivative.

17. The method of claim 16, wherein the retinoid derivative is acitretin, alitretinoin bexarotene, isotretinoin, retinol, retinoic acid, or a pharmaceutically acceptable salt thereof.

18. The method of claim 1, wherein the retinoid derivative is retinoic acid.

19. The method of claim 1, wherein the arginine depleting agent is co-administered with a therapeutically effective amount of an (−)-epigallocatechin-3-gallate (EGCG) derivative, a green tea catechin or a pharmaceutically acceptable salt or product thereof.

20. The method of claim 19, wherein the EGCG derivative is EGCG or pharmaceutically acceptable salt thereof or EGCG peracetate.

21. The method of claim 1, wherein the arginine depleting agent is co-administered with a therapeutically effective amount of a rapamycin derivative or pharmaceutically acceptable salt thereof.


SEQUENCE LISTING (107 sequences)

SEQ ID NO: 1 - DNA, 50 bp, Artificial Sequence
  ABD Primer 1, Synthesized in the Lab
ggagatggacatatgcatcatcaccatcaccatgatgaagccgtggatgc

SEQ ID NO: 2 - DNA, 50 bp, Artificial Sequence
  ABD Primer 2, Synthesized in the Lab
gctaagactttagcttcagctaaggaattcgcatccacggcttcatcatg

SEQ ID NO: 3 - DNA, 50 bp, Artificial Sequence
  ABD Primer 3, Synthesized in the Lab
ccttagctgaagctaaagtcttagctaacagagaacttgacaaatatgga

SEQ ID NO: 4 - DNA, 50 bp, Artificial Sequence
  ABD Primer 4, Synthesized in the Lab
taggttcttgtaatagtcacttactccatatttgtcaagttctctgttag

SEQ ID NO: 5 - DNA, 50 bp, Artificial Sequence
  ABD Primer 5, Synthesized in the Lab
ggagtaagtgactattacaagaacctaatcaacaatgccaaaactgttga

SEQ ID NO: 6 - DNA, 50 bp, Artificial Sequence
  ABD Primer 6, Synthesized in the Lab
aaatttcatctatcagtgcttttacaccttcaacagttttggcattgttg

SEQ ID NO: 7 - DNA, 50 bp, Artificial Sequence
  ABD Primer 7, Synthesized in the Lab
gtaaaagcactgatagatgaaattttagctgcattaccttcgggtagtaa

SEQ ID NO: 8 - DNA, 50 bp, Artificial Sequence
  ABD Primer 8, Synthesized in the Lab
tccgccgctaccattgttattattgttgttactacccgaaggtaatgcag

SEQ ID NO: 9 - DNA, 47 bp, Artificial Sequence
  ABD Primer 9, Synthesized in the Lab
gatcttaagcatatgcatcatcaccatcaccatgatgaagcggtgga

SEQ ID NO: 10 - DNA, 47 bp, Artificial Sequence
  ABD Primer 10, Synthesized in the Lab
gcttctttcgcttccgccaggctgttcgcatccaccgcttcatcatg

SEQ ID NO: 11 - DNA, 50 bp, Artificial Sequence
  ABD Primer 11, Synthesized in the Lab
gcggaagcgaaagaagcggcgaacgcggaactggatagctatggcgtgag

SEQ ID NO: 12 - DNA, 50 bp, Artificial Sequence
  ABD Primer 12, Synthesized in the Lab
ttatcaatcaggcgtttataaaaatcgctcacgccatagctatccagttc

SEQ ID NO: 13 - DNA, 50 bp, Artificial Sequence
  ABD Primer 13, Synthesized in the Lab
gatttttataaacgcctgattgataaagcgaaaaccgtggaaggcgtgga

SEQ ID NO: 14 - DNA, 51 bp, Artificial Sequence
  ABD Primer 14, Synthesized in the Lab
cggcagcgccgccagaatcgcatctttcagcgcttccacgccttccacggt

SEQ ID NO: 15 - DNA, 50 bp, Artificial Sequence
  ABD Primer 15, Synthesized in the Lab
ttctggcggcgctgccgtcgggtagtaacaacaataataacaatggtagc

SEQ ID NO: 16 - DNA, 39 bp, Artificial Sequence
  ABD Primer 16, Synthesized in the Lab
ggatccgccgctaccattgttattattgttgttactacc

SEQ ID NO: 17 - DNA, 50 bp, Artificial Sequence
  ABD Primer 17, Synthesized in the Lab
catatgttagctgatggatccagtaacaacaataataacaatggtagcgg

SEQ ID NO: 18 - DNA, 50 bp, Artificial Sequence
  ABD Primer 18, Synthesized in the Lab
aattcgcatccacggcttcatcaccgccgctaccattgttattattgttg

SEQ ID NO: 19 - DNA, 50 bp, Artificial Sequence
  ABD Primer 19, Synthesized in the Lab
gaagccgtggatgcgaattccttagctgaagctaaagtcttagctaacag

SEQ ID NO: 20 - DNA, 50 bp, Artificial Sequence
  ABD Primer 20, Synthesized in the Lab
cttactccatatttgtcaagttctctgttagctaagactttagcttcagc

SEQ ID NO: 21 - DNA, 50 bp, Artificial Sequence
  ABD Primer 21, Synthesized in the Lab
gagaacttgacaaatatggagtaagtgactattacaagaacctaatcaac

SEQ ID NO: 22 - DNA, 50 bp, Artificial Sequence
  ABD Primer 22,  Synthesized in the Lab
tacaccttcaacagttttggcattgttgattaggttcttgtaatagtcac

SEQ ID NO: 23 - DNA, 50 bp, Artificial Sequence
  ABD Primer 23, Synthesized in the Lab
gccaaaactgttgaaggtgtaaaagcactgatagatgaaattttagctgc

SEQ ID NO: 24 - DNA, 50 bp, Artificial Sequence
  ABD Primer 24, Synthesized in the Lab
atggtgatggtgatgatgaggtaatgcagctaaaatttcatctatcagtg

SEQ ID NO: 25 - DNA, 50 bp, Artificial Sequence
  ABD Primer 25, Synthesized in the Lab
catatgttatgcgatggatccagtaacaacaataataacaatggtagcgg

SEQ ID NO: 26 - DNA, 50 bp, Artificial Sequence
  ABD Primer 26, Synthesized in the Lab
tgttcgcatccaccgcttcatcaccgccgctaccattgttattattgttg

SEQ ID NO: 27 - DNA, 50 bp, Artificial Sequence
  ABD Primer 27, Synthesized in the Lab
gaagcggtggatgcgaacagcctggcggaagcgaaagaagcggcgaacgc

SEQ ID NO: 28 - DNA, 50 bp, Artificial Sequence
  ABD Primer 28, Synthesized in the Lab
aaaatcgctcacgccatagctatccagttccgcgttcgccgcttctttcg

SEQ ID NO: 29 - DNA, 50 bp, Artificial Sequence
  ABD Primer 29, Synthesized in the Lab
ctatggcgtgagcgatttttataaacgcctgattgataaagcgaaaaccg

SEQ ID NO: 30 - DNA, 50 bp, Artificial Sequence
  ABD Primer 30, Synthesized in the Lab
tcgcatctttcagcgcttccacgccttccacggttttcgctttatcaatc

SEQ ID NO: 31 - DNA, 50 bp, Artificial Sequence
  ABD Primer 31, Synthesized in the Lab
gaagcgctgaaagatgcgattctggcggcgctgccgcatcatcaccatca

SEQ ID NO: 32 - DNA, 35 bp, Artificial Sequence
  ABD Primer 32, Synthesized in the Lab
ctaaaagcttaatggtgatggtgatgatgcggcag

SEQ ID NO: 33 - DNA, 222 bp, Artificial Sequence
  His-ABD-Linker, Synthesized in the Lab
atgcatcatcaccatcaccatgatgaagccgtggatgcgaattccttagctgaagctaaagtcttagctaacagagaacttgacaaatatggagtaagtgactattacaagaacctaatcaacaatgccaaaactgttgaaggtgtaaaagcactgatagatgaaattttagctgcattaccttcgggtagtaacaacaataataacaatggtagcggcgga

SEQ ID NO: 34 - DNA, 222 bp, Artificial Sequence
  His-ABD094-Linker, Synthesized in the Lab
atgcatcatcaccatcaccatgatgaagcggtggatgcgaacagcctggcggaagcgaaagaagcggcgaacgcggaactggatagctatggcgtgagcgatttttataaacgcctgattgataaagcgaaaaccgtggaaggcgtggaagcgctgaaagatgcgattctggcggcgctgccgtcgggtagtaacaacaataataacaatggtagcggcgga

SEQ ID NO: 35 - DNA, 222 bp, Artificial Sequence
  Linker-ABD-His, Synthesized in the Lab
ggatccagtaacaacaataataacaatggtagcggcggtgatgaagccgtggatgcgaattccttagctgaagctaaagtcttagctaacagagaacttgacaaatatggagtaagtgactattacaagaacctaatcaacaatgccaaaactgttgaaggtgtaaaagcactgatagatgaaattttagctgcattacctcatcatcaccatcaccattaa

SEQ ID NO: 36 - DNA, 222 bp, Artificial Sequence
  Linker-ABD094-His, Synthesized in the Lab
ggatccagtaacaacaataataacaatggtagcggcggtgatgaagcggtggatgcgaacagcctggcggaagcgaaagaagcggcgaacgcggaactggatagctatggcgtgagcgatttttataaacgcctgattgataaagcgaaaaccgtggaaggcgtggaagcgctgaaagatgcgattctggcggcgctgccgcatcatcaccatcaccattaa

SEQ ID NO: 37 - DNA, 1191 bp, Artificial Sequence
  N-ABD-rhArg gene, Synthesized in the Lab
atgcatcatcaccatcaccatgatgaagccgtggatgcgaattccttagctgaagctaaagtcttagctaacagagaacttgacaaatatggagtaagtgactattacaagaacctaatcaacaatgccaaaactgttgaaggtgtaaaagcactgatagatgaaattttagctgcattaccttcgggtagtaacaacaataataacaatggtagcggcggaatgtccgccaagtccagaaccatagggattattggagctcctttctcaaagggacagccacgaggaggggtggaagaaggccctacagtattgagaaaggctggtctgcttgagaaacttaaagaacaagagtgtgatgtgaaggattatggggacctgccctttgctgacatccctaatgacagtccctttcaaattgtgaagaatccaaggtctgtgggaaaagcaagcgagcagctggctggcaaggtggcagaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaa

SEQ ID NO: 38 - DNA, 1191 bp, Artificial Sequence
  N-ABD094-rhArg gene, Synthesized in the Lab
atgcatcatcaccatcaccatgatgaagcggtggatgcgaacagcctggcggaagcgaaagaagcggcgaacgcggaactggatagctatggcgtgagcgatttttataaacgcctgattgataaagcgaaaaccgtggaaggcgtggaagcgctgaaagatgcgattctggcggcgctgccgtcgggtagtaacaacaataataacaatggtagcggcggaatgtccgccaagtccagaaccatagggattattggagctcctttctcaaagggacagccacgaggaggggtggaagaaggccctacagtattgagaaaggctggtctgcttgagaaacttaaagaacaagagtgtgatgtgaaggattatggggacctgccctttgctgacatccctaatgacagtccctttcaaattgtgaagaatccaaggtctgtgggaaaagcaagcgagcagctggctggcaaggtggcagaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaa

SEQ ID NO: 39 - DNA, 1188 bp, Artificial Sequence
  C-ABD-rhArg gene, Synthesized in the Lab
atgagcgccaagtccagaaccatagggattattggagctcctttctcaaagggacagccacgaggaggggtggaagaaggccctacagtattgagaaaggctggtctgcttgagaaacttaaagaacaagagtgtgatgtgaaggattatggggacctgccctttgctgacatccctaatgacagtccctttcaaattgtgaagaatccaaggtctgtgggaaaagcaagcgagcagctggctggcaaggtggcagaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagggatccagtaacaacaataataacaatggtagcggcggtgatgaagccgtggatgcgaattccttagctgaagctaaagtcttagctaacagagaacttgacaaatatggagtaagtgactattacaagaacctaatcaacaatgccaaaactgttgaaggtgtaaaagcactgatagatgaaattttagctgcattacctcatcatcaccatcaccattaa

SEQ ID NO: 40 - DNA, 1188 bp, Artificial Sequence
  C-ABD094-rhArg gene, Synthesized in the Lab
atgagcgccaagtccagaaccatagggattattggagctcctttctcaaagggacagccacgaggaggggtggaagaaggccctacagtattgagaaaggctggtctgcttgagaaacttaaagaacaagagtgtgatgtgaaggattatggggacctgccctttgctgacatccctaatgacagtccctttcaaattgtgaagaatccaaggtctgtgggaaaagcaagcgagcagctggctggcaaggtggcagaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagggatccagtaacaacaataataacaatggtagcggcggtgatgaagcggtggatgcgaacagcctggcggaagcgaaagaagcggcgaacgcggaactggatagctatggcgtgagcgatttttataaacgcctgattgataaagcgaaaaccgtggaaggcgtggaagcgctgaaagatgcgattctggcggcgctgccgcatcatcaccatcaccattaa

SEQ ID NO: 41 - DNA, 1125 bp, Artificial Sequence
  N-ABD-BCA gene, Synthesized in the Lab
atgcatcatcaccatcaccatgatgaagccgtggatgcgaattccttagctgaagctaaagtcttagctaacagagaacttgacaaatatggagtaagtgactattacaagaacctaatcaacaatgccaaaactgttgaaggtgtaaaagcactgatagatgaaattttagctgcattaccttcgggtagtaacaacaataataacaatggtagcggcggatccatgaagccaatttcaattatcggggttccgatggatttagggcagacacgccgcggcgttgatatggggccgagcgcaatgcgttatgcaggcgtcatcgaacgtctggaacgtcttcattacgatattgaagatttgggagatattccgattggaaaagcagagcggttgcacgagcaaggagattcacggttgcgcaatttgaaagcggttgcggaagcgaacgagaaacttgcggcggcggttgaccaagtcgttcagcgggggcgatttccgcttgtgttgggcggcgaccatagcatcgccattggcacgctcgccggggtggcgaaacattatgagcggcttggagtgatctggtatgacgcgcatggcgacgtcaacaccgcggaaacgtcgccgtctggaaacattcatggcatgccgctggcggcgagcctcgggtttggccatccggcgctgacgcaaatcggcggatacagccccaaaatcaagccggaacatgtcgtgttgatcggcgtccgttcccttgatgaaggggagaagaagtttattcgcgaaaaaggaatcaaaatttacacgatgcatgaggttgatcggctcggaatgacaagggtgatggaagaaacgatcgcctatttaaaagaacgaacggatggcgttcatttgtcgcttgacttggatggccttgacccaagcgacgcaccgggagtcggaacgcctgtcattggaggattgacataccgcgaaagccatttggcgatggagatgctggccgaggcacaaatcatcacttcagcggaatttgtcgaagtgaacccgatcttggatgagcggaacaaaacagcatcagtggctgtagcgctgatggggtcgttgtttggtgaaaaactcatgtaa

SEQ ID NO: 42 - DNA, 1125 bp, Artificial Sequence
  N-ABD094-BCA gene, Synthesized in the Lab
atgcatcatcaccatcaccatgatgaagcggtggatgcgaacagcctggcggaagcgaaagaagcggcgaacgcggaactggatagctatggcgtgagcgatttttataaacgcctgattgataaagcgaaaaccgtggaaggcgtggaagcgctgaaagatgcgattctggcggcgctgccgtcgggtagtaacaacaataataacaatggtagcggcggatccatgaagccaatttcaattatcggggttccgatggatttagggcagacacgccgcggcgttgatatggggccgagcgcaatgcgttatgcaggcgtcatcgaacgtctggaacgtcttcattacgatattgaagatttgggagatattccgattggaaaagcagagcggttgcacgagcaaggagattcacggttgcgcaatttgaaagcggttgcggaagcgaacgagaaacttgcggcggcggttgaccaagtcgttcagcgggggcgatttccgcttgtgttgggcggcgaccatagcatcgccattggcacgctcgccggggtggcgaaacattatgagcggcttggagtgatctggtatgacgcgcatggcgacgtcaacaccgcggaaacgtcgccgtctggaaacattcatggcatgccgctggcggcgagcctcgggtttggccatccggcgctgacgcaaatcggcggatacagccccaaaatcaagccggaacatgtcgtgttgatcggcgtccgttcccttgatgaaggggagaagaagtttattcgcgaaaaaggaatcaaaatttacacgatgcatgaggttgatcggctcggaatgacaagggtgatggaagaaacgatcgcctatttaaaagaacgaacggatggcgttcatttgtcgcttgacttggatggccttgacccaagcgacgcaccgggagtcggaacgcctgtcattggaggattgacataccgcgaaagccatttggcgatggagatgctggccgaggcacaaatcatcacttcagcggaatttgtcgaagtgaacccgatcttggatgagcggaacaaaacagcatcagtggctgtagcgctgatggggtcgttgtttggtgaaaaactcatgtaa

SEQ ID NO: 43 - DNA, 1119 bp, Artificial Sequence
  C-ABD-BCA gene, Synthesized in the Lab
atgaagccaatttcaattatcggggttccgatggatttagggcagacacgccgcggcgttgatatggggccgagcgcaatgcgttatgcaggcgtcatcgaacgtctggaacgtcttcattacgatattgaagatttgggagatattccgattggaaaagcagagcggttgcacgagcaaggagattcacggttgcgcaatttgaaagcggttgcggaagcgaacgagaaacttgcggcggcggttgaccaagtcgttcagcgggggcgatttccgcttgtgttgggcggcgaccatagcatcgccattggcacgctcgccggggtggcgaaacattatgagcggcttggagtgatctggtatgacgcgcatggcgacgtcaacaccgcggaaacgtcgccgtctggaaacattcatggcatgccgctggcggcgagcctcgggtttggccatccggcgctgacgcaaatcggcggatacagccccaaaatcaagccggaacatgtcgtgttgatcggcgtccgttcccttgatgaaggggagaagaagtttattcgcgaaaaaggaatcaaaatttacacgatgcatgaggttgatcggctcggaatgacaagggtgatggaagaaacgatcgcctatttaaaagaacgaacggatggcgttcatttgtcgcttgacttggatggccttgacccaagcgacgcaccgggagtcggaacgcctgtcattggaggattgacataccgcgaaagccatttggcgatggagatgctggccgaggcacaaatcatcacttcagcggaatttgtcgaagtgaacccgatcttggatgagcggaacaaaacagcatcagtggctgtagcgctgatggggtcgttgtttggtgaaaaactcatgggatccagtaacaacaataataacaatggtagcggcggtgatgaagccgtggatgcgaattccttagctgaagctaaagtcttagctaacagagaacttgacaaatatggagtaagtgactattacaagaacctaatcaacaatgccaaaactgttgaaggtgtaaaagcactgatagatgaaattttagctgcattacctcatcatcaccatcaccattaa

SEQ ID NO: 44 - DNA, 1119 bp, Artificial Sequence
  C-ABD094-BCA gene, Synthesized in the Lab
atgaagccaatttcaattatcggggttccgatggatttagggcagacacgccgcggcgttgatatggggccgagcgcaatgcgttatgcaggcgtcatcgaacgtctggaacgtcttcattacgatattgaagatttgggagatattccgattggaaaagcagagcggttgcacgagcaaggagattcacggttgcgcaatttgaaagcggttgcggaagcgaacgagaaacttgcggcggcggttgaccaagtcgttcagcgggggcgatttccgcttgtgttgggcggcgaccatagcatcgccattggcacgctcgccggggtggcgaaacattatgagcggcttggagtgatctggtatgacgcgcatggcgacgtcaacaccgcggaaacgtcgccgtctggaaacattcatggcatgccgctggcggcgagcctcgggtttggccatccggcgctgacgcaaatcggcggatacagccccaaaatcaagccggaacatgtcgtgttgatcggcgtccgttcccttgatgaaggggagaagaagtttattcgcgaaaaaggaatcaaaatttacacgatgcatgaggttgatcggctcggaatgacaagggtgatggaagaaacgatcgcctatttaaaagaacgaacggatggcgttcatttgtcgcttgacttggatggccttgacccaagcgacgcaccgggagtcggaacgcctgtcattggaggattgacataccgcgaaagccatttggcgatggagatgctggccgaggcacaaatcatcacttcagcggaatttgtcgaagtgaacccgatcttggatgagcggaacaaaacagcatcagtggctgtagcgctgatggggtcgttgtttggtgaaaaactcatgggatccagtaacaacaataataacaatggtagcggcggtgatgaagcggtggatgcgaacagcctggcggaagcgaaagaagcggcgaacgcggaactggatagctatggcgtgagcgatttttataaacgcctgattgataaagcgaaaaccgtggaaggcgtggaagcgctgaaagatgcgattctggcggcgctgccgcatcatcaccatcaccattaa

SEQ ID NO: 45 - protein, 74 aa, Artificial Sequence
  His-ABD-Linker, Synthesized in the Lab
MHHHHHHDEAVDANSLAEAKVLANRELDKYGVSDYYKNLINNAKTVEGVKALIDEILAALPSGSNNNNNNGSGG

SEQ ID NO: 46 - protein, 74 aa, Artificial Sequence
  His-ABD094-Linker, Synthesized in the Lab
MHHHHHHDEAVDANSLAEAKEAANAELDSYGVSDFYKRLIDKAKTVEGVEALKDAILAALPSGSNNNNNNGSGG

SEQ ID NO: 47 - protein, 73 aa, Artificial Sequence
  Linker-ABD-His, Synthesized in the Lab
GSSNNNNNNGSGGDEAVDANSLAEAKVLANRELDKYGVSDYYKNLINNAKTVEGVKALIDEILAALPHHHHHH

SEQ ID NO: 48 - protein, 73 aa, Artificial Sequence
  Linker-ABD094-His, Synthesized in the Lab
GSSNNNNNNGSGGDEAVDANSLAEAKEAANAELDSYGVSDFYKRLIDKAKTVEGVEALKDAILAALPHHHHHH

SEQ ID NO: 49 - protein, 396 aa, Artificial Sequence
  N-ABD-rhArg, Synthesized in the Lab
MHHHHHHDEAVDANSLAEAKVLANRELDKYGVSDYYKNLINNAKTVEGVKALIDEILAALPSGSNNNNNNGSGGMSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 50 - protein, 396 aa, Artificial Sequence
  N-ABD094-rhArg, Synthesized in the Lab
MHHHHHHDEAVDANSLAEAKEAANAELDSYGVSDFYKRLIDKAKTVEGVEALKDAILAALPSGSNNNNNNGSGGMSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 51 - protein, 395 aa, Artificial Sequence
  C-ABD-rhArg, Synthesized in the Lab
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPKGSSNNNNNNGSGGDEAVDANSLAEAKVLANRELDKYGVSDYYKNLINNAKTVEGVKALIDEILAALPHHHHHH

SEQ ID NO: 52 - protein, 395 aa, Artificial Sequence
  C-ABD094-rhArg, Synthesized in the Lab
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPKGSSNNNNNNGSGGDEAVDANSLAEAKEAANAELDSYGVSDFYKRLIDKAKTVEGVEALKDAILAALPHHHHHH

SEQ ID NO: 53 - protein, 374 aa, Artificial Sequence
  N-ABD-BCA, Synthesized in the Lab
MHHHHHHDEAVDANSLAEAKVLANRELDKYGVSDYYKNLINNAKTVEGVKALIDEILAALPSGSNNNNNNGSGGSMKPISIIGVPMDLGQTRRGVDMGPSAMRYAGVIERLERLHYDIEDLGDIPIGKAERLHEQGDSRLRNLKAVAEANEKLAAAVDQVVQRGRFPLVLGGDHSIAIGTLAGVAKHYERLGVIWYDAHGDVNTAETSPSGNIHGMPLAASLGFGHPALTQIGGYSPKIKPEHVVLIGVRSLDEGEKKFIREKGIKIYTMHEVDRLGMTRVMEETIAYLKERTDGVHLSLDLDGLDPSDAPGVGTPVIGGLTYRESHLAMEMLAEAQIITSAEFVEVNPILDERNKTASVAVALMGSLFGEKLM

SEQ ID NO: 54 - protein, 374 aa, Artificial Sequence
  N-ABD094-BCA, Synthesized in the Lab
MHHHHHHDEAVDANSLAEAKEAANAELDSYGVSDFYKRLIDKAKTVEGVEALKDAILAALPSGSNNNNNNGSGGSMKPISIIGVPMDLGQTRRGVDMGPSAMRYAGVIERLERLHYDIEDLGDIPIGKAERLHEQGDSRLRNLKAVAEANEKLAAAVDQVVQRGRFPLVLGGDHSIAIGTLAGVAKHYERLGVIWYDAHGDVNTAETSPSGNIHGMPLAASLGFGHPALTQIGGYSPKIKPEHVVLIGVRSLDEGEKKFIREKGIKIYTMHEVDRLGMTRVMEETIAYLKERTDGVHLSLDLDGLDPSDAPGVGTPVIGGLTYRESHLAMEMLAEAQIITSAEFVEVNPILDERNKTASVAVALMGSLFGEKLM

SEQ ID NO: 55 - protein, 372 aa, Artificial Sequence
  C-ABD-BCA, Synthesized in the Lab
MKPISIIGVPMDLGQTRRGVDMGPSAMRYAGVIERLERLHYDIEDLGDIPIGKAERLHEQGDSRLRNLKAVAEANEKLAAAVDQVVQRGRFPLVLGGDHSIAIGTLAGVAKHYERLGVIWYDAHGDVNTAETSPSGNIHGMPLAASLGFGHPALTQIGGYSPKIKPEHVVLIGVRSLDEGEKKFIREKGIKIYTMHEVDRLGMTRVMEETIAYLKERTDGVHLSLDLDGLDPSDAPGVGTPVIGGLTYRESHLAMEMLAEAQIITSAEFVEVNPILDERNKTASVAVALMGSLFGEKLMGSSNNNNNNGSGGDEAVDANSLAEAKVLANRELDKYGVSDYYKNLINNAKTVEGVKALIDEILAALPHHHHHH

SEQ ID NO: 56 - protein, 372 aa, Artificial Sequence
  C-ABD094-BCA, Synthesized in the Lab
MKPISIIGVPMDLGQTRRGVDMGPSAMRYAGVIERLERLHYDIEDLGDIPIGKAERLHEQGDSRLRNLKAVAEANEKLAAAVDQVVQRGRFPLVLGGDHSIAIGTLAGVAKHYERLGVIWYDAHGDVNTAETSPSGNIHGMPLAASLGFGHPALTQIGGYSPKIKPEHVVLIGVRSLDEGEKKFIREKGIKIYTMHEVDRLGMTRVMEETIAYLKERTDGVHLSLDLDGLDPSDAPGVGTPVIGGLTYRESHLAMEMLAEAQIITSAEFVEVNPILDERNKTASVAVALMGSLFGEKLMGSSNNNNNNGSGGDEAVDANSLAEAKEAANAELDSYGVSDFYKRLIDKAKTVEGVEALKDAILAALPHHHHHH

SEQ ID NO: 57 - DNA, 50 bp, Artificial Sequence
  ABD094-0F Primer, Synthesized in the Lab
attctggcagcgctgccgtcgggtagtaacaacaataataacaatggtag

SEQ ID NO: 58 - DNA, 50 bp, Artificial Sequence
  ABD094-1F Primer, Synthesized in the Lab
aaccgtggaaggtgtggaagcgctgaaagatgcgattctggcagcgctgc

SEQ ID NO: 59 - DNA, 50 bp, Artificial Sequence
  ABD094-2F Primer, Synthesized in the Lab
gatttttataaacgtctgattgataaagcgaaaaccgtggaaggtgtgga

SEQ ID NO: 60 - DNA, 50 bp, Artificial Sequence
  ABD094-3F Primer, Synthesized in the Lab
ctggatagctatggcgtgagcgatttttataaacgtctgattgataaagc

SEQ ID NO: 61 - DNA, 50 bp, Artificial Sequence
  ABD094-4F Primer, Synthesized in the Lab
gcggaagcgaaagaagcagcaaacgcggaactggatagctatggcgtgag

SEQ ID NO: 62 - DNA, 50 bp, Artificial Sequence
  ABD094-5F Primer, Synthesized in the Lab
accatgatgaagccgtggatgcgaattccctggcggaagcgaaagaagca

SEQ ID NO: 63 - DNA, 46 bp, Artificial Sequence
  abdNde-F Primer, Synthesized in the Lab
ggagatatacatatgcatcatcaccatcaccatgatgaagccgtgg

SEQ ID NO: 64 - DNA, 42 bp, Artificial Sequence
  HuArgHinBam-R Primer, Synthesized in the Lab
tgactaccttaacccacctaagtaagcttggatcctgtaacg

SEQ ID NO: 65 - DNA, 1220 bp, Artificial Sequence
  N-ABD094-rhArg gene, Synthesized in the Lab
ggagatatacatatgcatcatcaccatcaccatgatgaagccgtggatgcgaattccctggcggaagcgaaagaagcagcaaacgcggaactggatagctatggcgtgagcgatttttataaacgtctgattgataaagcgaaaaccgtggaaggtgtggaagcgctgaaagatgcgattctggcagcgctgccgtcgggtagtaacaacaataataacaatggtagcggcggtatgagcgccaagtccagaaccatagggattattggagctcctttctcaaagggacagccacgaggaggggtggaagaaggccctacagtattgagaaaggctggtctgcttgagaaacttaaagaacaagagtgtgatgtgaaggattatggggacctgccctttgctgacatccctaatgacagtccctttcaaattgtgaagaatccaaggtctgtgggaaaagcaagcgagcagctggctggcaaggtggcagaagtcaagaagaacggaagaatcagcctggtgctgggcggagaccacagtttggcaattggaagcatctctggccatgccagggtccaccctgatcttggagtcatctgggtggatgctcacactgatatcaacactccactgacaaccacaagtggaaacttgcatggacaacctgtatctttcctcctgaaggaactaaaaggaaagattcccgatgtgccaggattctcctgggtgactccctgtatatctgccaaggatattgtgtatattggcttgagagacgtggaccctggggaacactacattttgaaaactctaggcattaaatacttttcaatgactgaagtggacagactaggaattggcaaggtgatggaagaaacactcagctatctactaggaagaaagaaaaggccaattcatctaagttttgatgttgacggactggacccatctttcacaccagctactggcacaccagtcgtgggaggtctgacatacagagaaggtctctacatcacagaagaaatctacaaaacagggctactctcaggattagatataatggaagtgaacccatccctggggaagacaccagaagaagtaactcgaacagtgaacacagcagttgcaataaccttggcttgtttcggacttgctcgggagggtaatcacaagcctattgactaccttaacccacctaagtaagcttggatcctgtaacg

SEQ ID NO: 66 - protein, 54 aa, Artificial Sequence
  ABD based on Streptococcus sp. G148, Synthesized in the Lab
DEAVDANSLAEAKVLANRELDKYGVSDYYKNLINNAKTVEGVKALIDEILAALP

SEQ ID NO: 67 - protein, 57 aa, Artificial Sequence
  ABD based on Streptococcus sp. G148, Synthesized in the Lab
AQHDEAVDANSLAEAKVLANRELDKYGVSDYYKNLINNAKTVEGVKALIDEILAALP

SEQ ID NO: 68 - protein, 46 aa, Artificial Sequence
  ABD094 Without Linker, Synthesized in Lab
LAEAKEAANAELDSYGVSDFYKRLIDKAKTVEGVEALKDAILAALP

SEQ ID NO: 69 - protein, 324 aa, Homo sapiens
  Xaa (1)..(1): Xaa at position one is either methionine or not present.
XAASAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 70 - protein, 299 aa, Bacillus caldovelox
MKPISIIGVPMDLGQTRRGVDMGPSAMRYAGVIERLERLHYDIEDLGDIPIGKAERLHEQGDSRLRNLKAVAEANEKLAAAVDQVVQRGRFPLVLGGDHSIAIGTLAGVAKHYERLGVIWYDAHGDVNTAETSPSGNIHGMPLAASLGFGHPALTQIGGYSPKIKPEHVVLIGVRSLDEGEKKFIREKGIKIYTMHEVDRLGMTRVMEETIAYLKERTDGVHLSLDLDGLDPSDAPGVGTPVIGGLTYRESHLAMEMLAEAQIITSAEFVEVNPILDERNKTASVAVALMGSLFGEKLM

SEQ ID NO: 71 - protein, 299 aa, Artificial Sequence
  BCA (S161C) Arginase, Synthesized in the Lab
MKPISIIGVPMDLGQTRRGVDMGPSAMRYAGVIERLERLHYDIEDLGDIPIGKAERLHEQGDSRLRNLKAVAEANEKLAAAVDQVVQRGRFPLVLGGDHSIAIGTLAGVAKHYERLGVIWYDAHGDVNTAETSPSGNIHGMPLAASLGFGHPALTQIGGYCPKIKPEHVVLIGVRSLDEGEKKFIREKGIKIYTMHEVDRLGMTRVMEETIAYLKERTDGVHLSLDLDGLDPSDAPGVGTPVIGGLTYRESHLAMEMLAEAQIITSAEFVEVNPILDERNKTASVAVALMGSLFGEKLM

SEQ ID NO: 72 - protein, 299 aa, Artificial Sequence
  BCA (V20P, S161C) Arginase, Synthesized in the Lab
MKPISIIGVPMDLGQTRRGPDMGPSAMRYAGVIERLERLHYDIEDLGDIPIGKAERLHEQGDSRLRNLKAVAEANEKLAAAVDQVVQRGRFPLVLGGDHSIAIGTLAGVAKHYERLGVIWYDAHGDVNTAETSPSGNIHGMPLAASLGFGHPALTQIGGYCPKIKPEHVVLIGVRSLDEGEKKFIREKGIKIYTMHEVDRLGMTRVMEETIAYLKERTDGVHLSLDLDGLDPSDAPGVGTPVIGGLTYRESHLAMEMLAEAQIITSAEFVEVNPILDERNKTASVAVALMGSLFGEKLM

SEQ ID NO: 73 - protein, 12 aa, Artificial Sequence
  Linker Sequence 1, Synthesized in the Lab
GSNNNNNNGSGG

SEQ ID NO: 74 - protein, 13 aa, Artificial Sequence
  Linker Sequence 2, Synthesized in the Lab
SGSNNNNNNGSGG

SEQ ID NO: 75 - protein, 362 aa, Artificial Sequence
  BHA Fusion Protein, Synthesized in the Lab
MKPISIIGVPMDLGQTRRGPDMGPSAMRYAGVIERLERLHYDIEDLGDIPIGKAERLHEQGDSRLRNLKAVAEANEKLAAAVDQVVQRGRFPLVLGGDHSIAIGTLAGVAKHYERLGVIWYDAHGDVNTAETSPSGNIHGMPLAASLGFGHPALTQIGGYCPKIKPEHVVLIGVRSLDEGEKKFIREKGIKIYTMHEVDRLGMTRVMEETIAYLKERTDGVHLSLDLDGLDPSDAPGVGTPVIGGLTYRESHLAMEMLAEAQIITSAEFVEVNPILDERNKTASVAVALMGSLFGEKLMHHHHHHAQHDEAVDANSLAEAKVLANRELDKYGVSDYYKNLINNAKTVEGVKALIDEILAALP

SEQ ID NO: 76 - protein, 362 aa, Artificial Sequence
  BAH Fusion Protein, Synthesized in the Lab
MKPISIIGVPMDLGQTRRGPDMGPSAMRYAGVIERLERLHYDIEDLGDIPIGKAERLHEQGDSRLRNLKAVAEANEKLAAAVDQVVQRGRFPLVLGGDHSIAIGTLAGVAKHYERLGVIWYDAHGDVNTAETSPSGNIHGMPLAASLGFGHPALTQIGGYCPKIKPEHVVLIGVRSLDEGEKKFIREKGIKIYTMHEVDRLGMTRVMEETIAYLKERTDGVHLSLDLDGLDPSDAPGVGTPVIGGLTYRESHLAMEMLAEAQIITSAEFVEVNPILDERNKTASVAVALMGSLFGEKLMAQHDEAVDANSLAEAKVLANRELDKYGVSDYYKNLINNAKTVEGVKALIDEILAALPHHHHHH

SEQ ID NO: 77 - DNA, 103 bp, Artificial Sequence
  5′-Sense-ABD Primer, Synthesized in Lab
gcgcagcatgatgaagccgtggatgcgaacagcttagctgaagctaaagtcttagctaacagagaacttgacaaatatggagtaagtgactattacaagaacc

SEQ ID NO: 78 - DNA, 98 bp, Artificial Sequence
  5′-Anti-sense-ABD Primer, Synthesized in the Lab
ttaaggtaatgcagctaaaatttcatctatcagtgcttttacaccttcaacagttttggcattgttgattaggttcttgtaatagtcacttactccat

SEQ ID NO: 79 - DNA, 35 bp, Artificial Sequence
  Forward Primer for Cloning ABD, Synthesized in the Lab
gcatcaccatcaccatcacgcgcagcatgatgaag

SEQ ID NO: 80 - DNA, 38 bp, Artificial Sequence
  Reverse Primer for Cloning ABD, Synthesized in the Lab
cgggatccttaaggtaatgcagctaaaatttcatctat

SEQ ID NO: 81 - DNA, 33 bp, Artificial Sequence
  Forward Primer for Cloning BCA, Synthesized in the Lab
ggaattcccatatgaagccaatttcaattatcg

SEQ ID NO: 82 - DNA, 20 bp, Artificial Sequence
  Reverse Primer for Cloning BCA, Synthesized in the Lab
gtgatggtgatggtgatgca

SEQ ID NO: 83 - DNA, 33 bp, Artificial Sequence; identical to SEQ ID NO: 81
  Forward Primer for Cloning BHA, Synthesized in the Lab

SEQ ID NO: 84 - DNA, 38 bp, Artificial Sequence; identical to SEQ ID NO: 80
  Reverse Primer for Cloning BHA, Synthesized in the Lab

SEQ ID NO: 85 - DNA, 39 bp, Artificial Sequence
  Forward Primer for Cloning ABD, Synthesized in the Lab
cgttgtttggtgaaaaactcatggcgcagcatgatgaag

SEQ ID NO: 86 - DNA, 56 bp, Artificial Sequence
  Reverse Primer for Cloning ABD, Synthesized in the Lab
cgggatccttagtgatggtgatggtgatgaggtaatgcagctaaaatttcatctat

SEQ ID NO: 87 - DNA, 33 bp, Artificial Sequence; identical to SEQ ID NO: 81
  Forward Primer for Cloning BAH, Syntesized in the Lab

SEQ ID NO: 88 - DNA, 56 bp, Artificial Sequence; identical to SEQ ID NO: 86
  Reverse Primer for Cloning BAH, Synthesized in the Lab

SEQ ID NO: 89 - protein, 305 aa, Artificial Sequence
  BCA (S161C) - His, Synthesized in Lab
MKPISIIGVPMDLGQTRRGVDMGPSAMRYAGVIERLERLHYDIEDLGDIPIGKAERLHEQGDSRLRNLKAVAEANEKLAAAVDQVVQRGRFPLVLGGDHSIAIGTLAGVAKHYERLGVIWYDAHGDVNTAETSPSGNIHGMPLAASLGFGHPALTQIGGYCPKIKPEHVVLIGVRSLDEGEKKFIREKGIKIYTMHEVDRLGMTRVMEETIAYLKERTDGVHLSLDLDGLDPSDAPGVGTPVIGGLTYRESHLAMEMLAEAQIITSAEFVEVNPILDERNKTASVAVALMGSLFGEKLMHHHHHH

SEQ ID NO: 90 - DNA, 42 bp, Artificial Sequence
  N-ABDHind-R Primer, Synthesized in the Lab
acagctaaaagcttatcagcctaggatccgccgctaccattg

SEQ ID NO: 91 - DNA, 38 bp, Artificial Sequence
  C-ABDNde-F Primer, Synthesized in the Lab
tagctgatcatatgttatgcgatggatccagtaacaac

SEQ ID NO: 92 - DNA, 33 bp, Artificial Sequence
  C-ABDHind-R, Synthesized in the Lab
gaccctaaaagcttaatggtgatggtgatgatg

SEQ ID NO: 93 - DNA, 34 bp, Artificial Sequence
  HARGBam-F Primer, Synthesized in the Lab
ttagctggggatccgccaagtccagaaccatagg

SEQ ID NO: 94 - DNA, 35 bp, Artificial Sequence
  HARGHind-R Primer, Synthesized in the Lab
gagatcaaagcttacttaggtgggttaaggtagtc

SEQ ID NO: 95 - DNA, 35 bp, Artificial Sequence
  HARGNde-F, Synthesized in the Lab
taggctgcatatgagcgccaagtccagaaccatag

SEQ ID NO: 96 - DNA, 41 bp, Artificial Sequence
  HARGBam-R Primer, Synthesized in the Lab
atcagctaggatcccttaggtgggttaaggtagtcaatagg

SEQ ID NO: 97 - DNA, 36 bp, Artificial Sequence
  BCABam-F Primer, Synthesized in the Lab
atgctagtggatccatgaagccaatttcaattatcg

SEQ ID NO: 98 - DNA, 38 bp, Artificial Sequence
  BCAHind-R Primer, Synthesized in the Lab
tcagcctaaagcttacatgagtttttcaccaaacaacg

SEQ ID NO: 99 - DNA, 35 bp, Artificial Sequence
  BCANde-F Primer, Synthesized in the Lab
atgctagccatatgaagccaatttcaattatcggg

SEQ ID NO: 100 - DNA, 37 bp, Artificial Sequence
  BCABam-R Primer, Synthesized in the Lab
tcagctaaggatcccatgagtttttcaccaaacaacg

SEQ ID NO: 101 - protein, 329 aa, Artificial Sequence
  His-rhArg, Synthesized in Lab
MHHHHHHMSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 102 - protein, 329 aa, Artificial Sequence
  His-rhArg-mono-Cys, Synthesized in Lab
MHHHHHHMSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPSISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLASFGLAREGNHKPIDYLNPPK

SEQ ID NO: 103 - protein, 322 aa, Homo sapiens
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 104 - protein, 322 aa, Artificial Sequence
  rhArg-mono-Cys, Synthesized in the Lab
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPSISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLASFGLAREGNHKPIDYLNPPK

SEQ ID NO: 105 - protein, 46 aa, Artificial Sequence
  A(EAAAK)4ALEA-(EAAAK)4A Peptide Linker, Synthesized in the Lab
AEAAAKEAAAKEAAAKEAAAKALEAEAAAKEAAAKEAAAKEAAAKA

SEQ ID NO: 106 - protein, 15 aa, Artificial Sequence
  G4SG4SG3SG Peptide Linker, Synthesized in the Lab
GGGGSGGGGSGGGSG

SEQ ID NO: 107 - protein, 483 aa, Artificial Sequence
  ADI-ABD, Synthesized in the Lab
MHHHHHHDEAVDANSLAEAKVLANRELDKYGVSDYYKNLINNAKTVEGVKALIDEILAALPSGSNNNNNNGSGGSVFDSKFKGIHVYSEIGELESVLVHEPGREIDYITPARLDELLFSAILESHDARKEHKQFVAELKANDINVVELIDLVAETYDLASQEAKDKLIEEFLEDSEPVLSEEHKVVVRNFLKAKKTSRELVEIMMAGITKYDLGIEADHELIVDPMPNLYFTRDPFASVGNGVTIHYMRYKVRQRETLFSRFVFSNHPKLINTPWYYDPSLKLSIEGGDVFIYNNDTLVVGVSERTDLQTVTLLAKNIVANKECEFKRIVAINVPKWTNLMHLDTWLTMLDKDKFLYSPIANDVFKFWDYDLVNGGAEPQPVENGLPLEGLLQSIINKKPVLIPIAGEGASQMEIERETHFDGTNYLAIRPGVVIGYSRNEKTNAALEAAGIKVLPFHGNQLSLGMGNARCMSMPLSRKDVKW